SEQUENCE LISTING (51 sequences)

SEQ ID NO: 1 - DNA, 1725 bp, Homo sapiens
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctctaa

SEQ ID NO: 2 - protein, 574 aa, Homo sapiens
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 3 - DNA, 34 bp, Artificial Sequence
  PCR primer Acb787
agagagggggcccaagaagatgacatcataattg

SEQ ID NO: 4 - DNA, 34 bp, Artificial Sequence
  PCR primer Acb786
ctgcgagtttaaactattaattagagacccacac

SEQ ID NO: 5 - DNA, 22 bp, Artificial Sequence
  PCR primer Acb710
gtgtaactctctttggagaaag

SEQ ID NO: 6 - DNA, 67 bp, Artificial Sequence
  PCR primer Acb853
tataagtttaaacatataattggatcctccacctccgcctccgagacccacacaactttctttcttg

SEQ ID NO: 7 - DNA, 40 bp, Artificial Sequence
  PCR primer Acb854
atataaggatccgatgcacacaagagtgaggttgctcatc

SEQ ID NO: 8 - DNA, 44 bp, Artificial Sequence
  PCR primer Acb855
atttaagtttaaactcattataagcctaaggcagcttgacttgc

SEQ ID NO: 9 - DNA, 77 bp, Artificial Sequence
  PCR primer Acb719
atattctcgagagccatgaaggtcctcatccttgcctgtctggtggctctggcccttgcaagagaagatgacatcat

SEQ ID NO: 10 - DNA, 40 bp, Artificial Sequence
  PCR primer Acb718
ctatgactcgaggcgatcgctattaattagagacccacac

SEQ ID NO: 11 - DNA, 20 bp, Artificial Sequence
  PCR primer ACB712
cttccgtggccagaatggat

SEQ ID NO: 12 - DNA, 27 bp, Artificial Sequence
  PCR primer ACB244
catcagaagttaaacagcacagttagt

SEQ ID NO: 13 - DNA, 24 bp, Artificial Sequence
  PCR primer ACB268
aggagcacagtgctcatccagatc

SEQ ID NO: 14 - DNA, 21 bp, Artificial Sequence
  PCR primer ACB659
gacgccccatcctcactgact

SEQ ID NO: 15 - DNA, 22 bp, Artificial Sequence
  PCR primer ACB572
ttcctaggatgtgctccaggct

SEQ ID NO: 16 - DNA, 22 bp, Artificial Sequence
  PCR primer ACB255
gaaacggaatgttgtggagtgg

SEQ ID NO: 17 - DNA, 24 bp, Artificial Sequence
  PCR primer Acb266
tgctctttgagcctgcagacacct

SEQ ID NO: 18 - DNA, 24 bp, Artificial Sequence
  PCR primer Acb267
ggctgttctgaacgctgtgacttg

SEQ ID NO: 19 - DNA, 19 bp, Artificial Sequence
  PCR primer Acb819
ccagaggtaaaccaaagac

SEQ ID NO: 20 - DNA, 20 bp, Artificial Sequence
  PCR primer ACB884
cctcactcttgtgtgcatcg

SEQ ID NO: 21 - DNA, 21 bp, Artificial Sequence
  PCR primer Acb256
gaggaacaacagcaaacagag

SEQ ID NO: 22 - DNA, 21 bp, Artificial Sequence
  PCR primer Acb312
accctactgtctttcatcagc

SEQ ID NO: 23 - DNA, 47 bp, Artificial Sequence
  PCR primer WAP-pl
aattggtaccagcggccgctctagaggaactgaagcagagaccatgc

SEQ ID NO: 24 - DNA, 87 bp, Artificial Sequence
  PCR primer WAP-p2
gctgctcgagcttgatgtttaaactgataacccttcagtgagcagccgatatatgtttaaacatgcgttgcctcatcagccttgttc

SEQ ID NO: 25 - DNA, 39 bp, Artificial Sequence
  PCR primer WAP-p3
atatatgtttaaacatgcgttgcctcatcagccttgttc

SEQ ID NO: 26 - DNA, 28 bp, Artificial Sequence
  PCR primer WAP-p4
atgttctctctggatccaggagtgaagg

SEQ ID NO: 27 - DNA, 18 bp, Artificial Sequence
  PCR primer BChE-pl
atttccccgaagtattac

SEQ ID NO: 28 - DNA, 18 bp, Artificial Sequence
  PCR primer BChE-p2
tgattttctgtggttatt

SEQ ID NO: 29 - DNA, 35 bp, Artificial Sequence
  PCR primer Linker-pl
ggaccggtgttaacgatatctctagagcggccgct

SEQ ID NO: 30 - DNA, 41 bp, Artificial Sequence
  PCR primer Linker-p2
ccggagcggccgctctagagatatcgttaacaccggtccgc

SEQ ID NO: 31 - DNA, 44 bp, Artificial Sequence
  PCR primer Insulator-pl
ttttgcggccgctctagactcgaggggacagcccccccccaaag

SEQ ID NO: 32 - DNA, 48 bp, Artificial Sequence
  PCR primer Insulator-p2
ttttggatccgtcgacgccccatcctcactgactccgtcctggagttg

SEQ ID NO: 33 - DNA, 32 bp, Artificial Sequence
  PCR primer Linker-p3
ggactagttgatcagcggccgctataggatcc

SEQ ID NO: 34 - DNA, 39 bp, Artificial Sequence
  PCR primer Linker-p4
ggcctggatcctatagcggccgctgatcaactagtccgc

SEQ ID NO: 35 - DNA, 35 bp, Artificial Sequence
  PCR sense primer
caatcaggccggccagaagatgacatcataattgc

SEQ ID NO: 36 - DNA, 39 bp, Artificial Sequence
  PCR sense primer
caatcaggcgcgccctcgaggatctcggccctctttctg

SEQ ID NO: 37 - DNA, 40 bp, Artificial Sequence
  PCR antisense primer
caatcaggccggccgcaatagagacctgcagtccccggag

SEQ ID NO: 38 - DNA, 35 bp, Artificial Sequence
  PCR sense primer
catctggcgatcgctaccgagtacagaaggggacg

SEQ ID NO: 39 - DNA, 36 bp, Artificial Sequence
  PCR antisense primer
ctagcatgcggccgcgtgctctaggacagccagagc

SEQ ID NO: 40 - protein, 16 aa, Artificial Sequence
  synthetic peptide
ESTGGGPPPPPPPPPP

SEQ ID NO: 41 - DNA, 30 bp, Artificial Sequence
  PCR primer ACB582
cagctagtattcatggaagggcaaatgagg

SEQ ID NO: 42 - DNA, 30 bp, Artificial Sequence
  PCR primer ACB591
tagaggtcagggatgctgctaaacattctg

SEQ ID NO: 43 - DNA, 30 bp, Artificial Sequence
  PCR primer ACB583
ccacagaattgactgcgactggaaatatgg

SEQ ID NO: 44 - DNA, 30 bp, Artificial Sequence
  PCR primer ACB601
ctccatgggtaagcctaaacattgagatct

SEQ ID NO: 45 - DNA, 28 bp, Artificial Sequence
  PCR primer ACB620
ctttctcagcccaaagttctgcctgttc

SEQ ID NO: 46 - DNA, 28 bp, Artificial Sequence
  PCR primer ACB621
caagttctctctcatctcctgcttctca

SEQ ID NO: 47 - DNA, 28 bp, Artificial Sequence
  PCR primer ACB618
cagtggacagaggaagagtcagaggaag

SEQ ID NO: 48 - DNA, 28 bp, Artificial Sequence
  PCR primer ACB619
gtatttacctctcttgcaagggccagag

SEQ ID NO: 49 - DNA, 3501 bp, Artificial Sequence
  DNA sequence encoding BChE-HSA fusion protein
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctcggaggcggaggtggaggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 50 - protein, 1166 aa, Artificial Sequence
  BChE-HSA fusion protein containing Gly/Ser linker
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLGGGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 51 - DNA, 50 bp, Artificial Sequence
  PCR antisense primer
ctatgactcgaggcgatcactcattataagcctaaggcagcttgacttgc

(19) United States
(12) Patent Application Publication
(10) Pub. No.: US 2006/0253913 A1
(43) Pub. Date: Nov. 9, 2006

(54) Production of hSA-linked butyrylcholinesterases in transgenic mammals

(76) Inventors: Yue-Jin Huang, Montreal (CA); Costas N. Karatzas, Beaconsfield (CA); Anthoula Lazaris, Beaconsfield (CA)

Correspondence Address: Alan J. Grant, Esq.;c/o Carella, Byrne, Bain, Gilfillan, Cecchi,, Stewart & Olstein, 5 Becker Farm Road, Roseland, NJ, 07068, US

(21) Appl. No.: 11/401,390

(22) Filed: Apr. 10, 2006

Related U.S. Application Data
(63) Continuation-in-part of application No. 10/326,892, filed on Dec. 20, 2002.
(60) Provisional application No. 60/344,295, filed on Dec. 21, 2001.

(51) Int. Cl. A01K 67/027; C07H 21/04; C12P 21/06; C12N 9/18; C12N 5/06

(52) U.S. Cl. 800/14; 800/18; 435/69.1; 435/197; 435/354; 435/455; 536/23.2

(57) ABSTRACT

The present invention provides methods for the large-scale production of recombinant butyrylcholinesterase fused to human serum albumin in cell culture, and in the milk and/or urine of transgenic mammals. The recombinant butyrylcholinesterase-albumin fusion protein of this invention can be used to treat and/or prevent organophosphate pesticide poisoning, nerve gas poisoning, cocaine intoxication, and succinylcholine-induced apnea.

This application is a continuation-in-part of U.S. application Ser. No. 10/326,892, filed 20 Dec. 2002, which claimed priority of U.S. Provisional Aopplication 60/344,295, filed 21 Dec. 2001, the disclosure of which is hereby incorporated by reference in its entirety.

### FIELD OF THE INVENTION

The present invention provides methods for the large-scale production of recombinant butyrylcholinesterase fused to human serum albumin in cell culture, and in the milk and/or urine of transgenic mammals. The recombinant butyrylcholinesterases of this invention can be used to treat and/or prevent organophosphate pesticide poisoning, nerve gas poisoning, cocaine intoxication, and succinylcholine-induced apnea.

### BACKGROUND OF THE INVENTION

The general term cholinesterase (ChE) refers to a family of enzymes involved in nerve impulse transmission and whose major function is to catalyze the hydrolysis of the chemical compound acetylcholine at the cholinergic synapses found throughout the nervous systems of humans, other vertebrates and insects. Stimulating signals are carried by the neurotransmitter acetylcholine, and discontinued by the action of ChE enzymes, which cause hydrolytic breakdown of acetylcholine. The action of ChE allows the muscle, gland, or nerve to return to its resting state, ready to receive another nerve impulse if need be.

Cholinesterase-inhibiting substances such as organophosphate compounds or carbamate insecticides or drugs prevent the breakdown of acetylcholine, resulting in a buildup of acetylcholine, thereby causing hyperactivity of the nervous system. Acetylcholine continues to stimulate the muscarinic receptor sites (exocrine glands and smooth muscles) and the nicotinic receptor sites (skeletal muscles). Exposure to cholinesterase-inhibiting substances can cause symptoms ranging from mild (twitching, trembling) to severe (paralyzed breathing, convulsions), and in extreme cases, death, depending on the type and amount of cholinesterase-inhibiting substances involved. This makes them very effective as pesticides for controlling insects and other pests. When humans breathe or are otherwise exposed to these compounds, they are subjected to the same negative effects, which has led to the development of these compounds as “nerve gases” or chemical warfare agents.

Cholinesterases are classified into two broad groups, depending on their substrate preference and sensitivity to selective inhibitors. Those enzymes which preferentially hydrolyze acetyl esters such as acetylcholine, and whose enzymatic activity is sensitive to the chemical inhibitor BW 284C51, are called acetylcholinesterases (AChE), or acetylcholine acetylhydrolase, (EC 3.1.1.7). Those enzymes which preferentially hydrolyze other types of esters such as butyrylcholine, and whose enzymatic acticity is sensitive to the chemical inhibitor tetraisopropylpyrophosphoramide (also known as iso-OMPA), are called butyrylcholinesterases (BChE, EC 3.1.1.8). BChE is also known as pseudocholinesterase or non-specific cholinesterase. Further classifications of ChE's are based on charge, hydrophobicity, interaction with membrane or extracellular structures, and subunit composition.

Acetylcholinesterase (AChE), also known as true, specific, genuine, erythrocyte, red cell, or Type I ChE, is a membrane-bound glycoprotein and exists in several molecular forms. It is found in erythrocytes, nerve endings, lungs, spleen, and the gray matter of the brain. Butyrylcholinesterase (BChE), also known as plasma, serum, benzoyl, false, or Type II ChE, has more than eleven isoenzyme variants and preferentially uses butyrylcholine and benzoylcholine as in vitro substrates. BChE is found in mammalian blood plasma, liver, pancreas, intestinal mucosa, the white matter of the central nervous system, smooth muscle, and heart. BChE is sometimes referred to as serum cholinesterase as opposed to red cell cholinesterase (AChE).

AChE and BChE exist in parallel arrays of multiple molecular forms composed of different numbers of catalytic and non-catalytic subunits. Both enzymes are composed of subunits of about 600 amino acids each, and both are glycosylated. AChE may be distinguished from the closely related BChE by its high specificity for the acetylcholine substrate and sensitivity to selective inhibitors. While AChE is primarily used in the body to hydrolyze acetylcholine, the specific function of BChE is not as clear. BChE has no known specific natural substrate, although it also hydrolyzes acetylcholine.

Poisoning with organophosphate agents is a severe problem facing military personnel who may encounter lethal doses of these compounds in chemical warfare situations. The use of organophosphate compounds in war and as pesticides has resulted over the past 40 years in a rising number of cases of acute and delayed intoxication, resulting in damage to the peripheral and central nervous systems, myopathy, psychosis, general paralysis, and death.

Nerve agents are the most toxic chemical warfare agents. These compounds are related to organophosphorus insecticides, in that they are both esters of phosphoric acid. The major nerve agents are GA (tabun), GB (sarin), GD (soman), GF, and VX. VX is a persistent substance which can remain on material, equipment, and terrain for long periods. Under temperate conditions, nerve agents are clear colorless liquids.

Nerve agents exert their biological activity by inhibiting the cholinesterase enzymes. In cases of moderate to severe organophospate poisoning, the levels of both AChE and BChE activity are reduced. Mild poisoning occurs when cholinesterase activity is 20-50% of normal; moderate poisoning occurs when activity is 10-20% of normal; severe poisoning is characterized by activity of less than 10% of normal. Severe neuromuscular effects are observed when ChE activity levels drop below 20% of normal, while levels near zero are generally fatal.

Present treatment of organophosphate poisoning consists of post-exposure intravenous or intramuscular administration of various combinations of drugs, including carbamates (e.g., pyridostigmine), anti-muscarinics (e.g., atropine), and ChE-reactivators such as pralidoxime chloride (2-PAM, Protopam). A diazopan compound may also be administered. Although this drug regimen is effective in preventing death from organophosphate poisoning, it is not effective in preventing convulsions, performance deficits, or permanent brain damage. In addition, a post-exposure drug regimen is often useless because even a small dose of an organophosphate chemical warfare agent can cause instant death. These drawbacks have led to the investigation of cholinesterase enzymes for the treatment of organophosphate exposure. Post-exposure symptoms can be prevented by pretreatment with cholinesterases, which act to sequester the toxic organophosphates before they reach their physiological targets.

The use of cholinesterases as pre-treatment drugs has been successfully demonstrated in animals, including non-human primates. For example, pretreatment of rhesus monkeys with fetal bovine serum-derived AChE or horse serum-derived BChE protected them against a challenge of two to five times the LD50 of pinacolyl methylphosphonofluoridate (soman), a highly toxic organophophate compound used as a war-gas [Broomfield, et al. J. Pharmacol. Exp. Ther. (1991) 259:633-638; Wolfe, et al. Toxicol Appl Pharmacol (1992) 117(2):189-193]. In addition to preventing lethality, the pretreatment prevented behavioral incapacitation after the soman challenge, as measured by the serial probe recognition task or the equilibrium platform performance task. Administration of sufficient exogenous human BChE can protect mice, rats, and monkeys from multiple lethal-dose organophosphate intoxication [see for example Raveh, et al. Biochemical Pharmacology (1993) 45:2465-2474; Raveh, 10 et al. Toxicol. Appl. Pharmacol. (1997) 145:43-53; Allon, et al. Toxicol. Sci. (1998) 43:121-128]. Purified human BChE has been used to treat organophosphate poisoning in humans, with no significant adverse immunological or psychological effects (Cascio, et al. Minerva Anestesiol (1998) 54:337).

Titration of organophosphates both in vitro and in vivo demonstrates a 1:1 stoichiometry between organophosphate-inhibited enzymes and the cumulative dose of the toxic nerve agent. The inhibition of ChE by an organophosphate agent is due to the formation of a stable stoichiometric (1:1) covalent conjugate of the organophosphate with the ChE active site serine. Covalent conjugation is followed by a parallel competing reaction, termed “aging”, wherein the inhibited ChE is transformed into a form that cannot be regenerated by the commonly used reactivators. These reactivators, such as active-site directed nucleophiles (e.g., quaternary oximes), normally detach the phosphoryl moiety from the hydroxyl group of the active site serine. The aging process is believed to involve dealkylation of the covalently bound organophosphate group, and renders therapy of intoxication by certain organophosphates such as sarin, soman, and DFP exceedingly difficult.

Despite the promise of cholinesterases as drugs to protect against organophosphate poisoning, their widespread use is not currently possible due to the limited supply of these enzymes. Because of the 1:1 stoichiometry required to provide protection, large quantities of cholinesterase enzymes are needed for effective treatment. The only practical source of these enzymes at present is by extraction from human plasma (see, e.g., U.S. Pat. No. 5,272,080 to Lynch). It is estimated that the number of doses needed for military purposes alone far exceeds the available supplies.

In addition to its efficacy in hydrolyzing organophosphate toxins, there is strong evidence that BChE is the major detoxifying enzyme of cocaine [Xie, et al. Molec. Pharmacol. (1999) 55:83-91]. Cocaine is metabolized by three major routes: hydrolysis by BChE to form ecgonine methyl ester, N-demethylation from norcocaine, and nonenzymatic hydrolysis to form benzoylcholine. Studies have shown a direct correlation between low BChE levels and episodes of life-threatening cocaine toxicity. A recent study has confirmed that a decrease of cocaine half-life in vitro correlated with the addition of purified human BChE.

In view of the significant pharmaceutical potential of ChE enzymes, research has focused on development of recombinant methods to produce them. Recombinant enzymes, as opposed to those derived from plasma, have a much lower risk of transmission of infectious agents, including viruses such as hepatitis C and HIV.

The cDNA sequences have been cloned for both human AChE (see U.S. Pat. No. 5,595,903) and human BChE [see U.S. Pat. No. 5,215,909 to Soreq; Prody, et al. Proc. Natl. Acad. Sci. USA (1987) 84:3555-3559; McTiernan, et al. Proc. Natl. Acad. Sci USA (1987) 84:6682-6686]. The amino acid sequence of wildtype human BChE, as well as of several BChE variants with single amino acid changes, is set forth in U.S. Pat. No. 6,001,625 to Broomfield, et al. Recombinant expression of BChE has been reported in E. coli [Masson, P., “Expression and Refolding of Functional Human BChE from E. coli,” Multiple Approaches to Cholinesterase Functions (Eds. Shafferaman, A and Velan, B.), Plenum, New York, 1990, pp. 49-52]; microinjected Xenopus laevis oocytes [U.S. Pat. No. 5,215,909 to Soreq; Soreq, H. et al., J. Biol. Chem. 264:10608-10613 (1989); Soreq, H et al. EMBO Journal 3(6):1371-1375 (1984)]; insect cell lines in vitro and larvae in vivo [Platteborze and Broomfield Biotechnol Appl Biochem 31:225-229 (2000)]; the silkworm Borbyx mori [Wei W. L., et al. Biochem Pharmacol 60(1):121-126 (2000)]; and in mammalian COS cells [Platteborze and Broomfield Biotechnol Appl Biochem 31:225-229 (2000)] and CHO cells [Masson, P. et al. J. Biol Chem 268(19):14329-41 (1993); Lockridge, O. et al., Biochemistry 36(4):786-795 (1997); Blong, R. et al. Biochem. J. 327:747-757 (1997); and Altamirano, C. V. et al. J. Neurochemistry 74:869-877 (2000)]. However, many of these reported recombinantly produced BChE preparations have thus far showed little or no in vivo enzyme activity.

Notably, none of the recombinant expression systems reported to date have the ability to produce BChE in quantities sufficient to allow development of the enzyme as a drug to treat such conditions as organophosphate poisoning, post-surgical apnea, or cocaine intoxication. However, an additional problem is longevity. Thus, the longer the BChE remains in the system of a person treated, the longer it is available for detoxification. Such lifespan is referred to as the “mean residence time” in the system and the present invention solves the problem of short lifespan by supplying a fusion protein comprising BChE and an additional protein and that promotes lifespan in the system as well as promoting full activity of the BChE portion of the fusion protein.

### BRIEF SUMMARY OF THE INVENTION

The present inventors have discovered methods for producing large quantities of recombinant butyrylcholinesterase (BChE) fused to human serum albumin (hSA) in the milk of lactating transgenic mammals, and in the urine of transgenic mammals. The methods of the invention for the first time allow sufficient quantities of the BChE-hSA fusion protein to be produced so as to permit practical development of this protein for prevention and/or treatment for organophosphorus poisoning, cocaine intoxication, and succinyl choline-induced apnea.

The present invention is directed to non-human transgenic mammals that upon lactation, express a BChE-hSA fusion protein in their milk, where the genomes of the mammals comprise a DNA sequence encoding a BChE-enzyme and a hSA protein, linked through a DNA linker that encodes at least 7 amino acid residues, preferably composed of 6 glycines and one serine in a sequential order, operably linked to a mammary gland-specific promoter, and a signal sequence that provides secretion of the BChE-hSA fusion protein into the milk of the mammal. The linker sequence is available to promote independent folding and activity of said BChE as well as the hSA. In preferred embodiments, the mammary gland-specific promoter is a casein promoter or a whey acidic protein (WAP) promoter. In preferred embodiments, the transgenic mammals are goats or rodents.

The present invention is also directed to non-human transgenic mammals that express a BChE-hSA fusion protein in their urine, where the genomes of the mammals comprise a DNA sequence encoding a BChE enzyme and a hSA protein, linked through a DNA linker that encodes at least 7 amino acid residues, preferably composed of 6 glycines and one serine in a sequential order, operably linked to a urinary endothelium-specific promoter, and a signal sequence that provides secretion of the BChE-hSA fusion protein into the urine of the mammal. In preferred embodiments, the urinary endothelium-specific promoter is a uroplakin promoter or a uromodulin promoter. In preferred embodiments, the transgenic mammals are goats or rodents.

In further embodiments, the invention is directed to such transgenic mammals, where the genomes of the mammals further comprise a DNA sequence encoding a glycosyltransferase, operably linked to a mammary gland-specific or a urinary endothelium-specific promoter, and a signal sequence that provides secretion of the glycosyltransferase. The BChE-hSA fusion protein and the glycosyltransferase may be encoded together in a single, bi-cistronic expression construct. Alternatively, the BChE-hSA fusion protein and the glycosyltransferase are encoded in separate expression constructs, which are both introduced into the genome of the mammal.

In another aspect the present invention is directed to a genetically-engineered DNA sequence, which comprises: (i) a sequence encoding a BChE-hSA fusion protein; (ii) a mammary gland-specific promoter that directs expression of the BChE-hSA fusion protein; and (iii) at least one signal sequence that provides secretion of the expressed BChE-hSA fusion protein. In preferred embodiments, the mammary gland-specific promoter is a WAP (whey acidic protein) promoter or a casein promoter. The invention also contemplates a non-human mammalian embryo or mammalian cell that comprises such a DNA sequence, especially where the cell is a MAC-T (mammary epithelial) cell, embryonic stem cell, embryonal carcinoma cell, primordial germ cell, oocyte, or sperm. The present invention is also directed to a method for making such a genetically-engineered DNA sequence, which method comprises joining a sequence encoding a BChE-hSA fusion protein with a mammary gland-specific promoter that directs expression of the BChE-hSA fusion protein and at least one signal sequence that provides secretion of the expressed BChE-hSA fusion protein.

In another aspect the present invention is directed to a genetically-engineered DNA sequence, which comprises: (i) a sequence encoding a BChE-hSA fusion protein; (ii) a urinary endothelium-specific promoter that directs expression of the BChE-hSA fusion protein; and (iii) at least one signal sequence that provides secretion of the expressed BChE-hSA fusion protein. In preferred embodiments, the urinary endothelium-specific promoter is a uroplakin promoter or a uromodulin promoter. The invention also contemplates a non-human mammalian embryo or mammalian cell that comprises such a DNA sequence, especially where the cell is a BHK (baby hamster kidney) cell, embryonic stem cell, embryonal carcinoma cell, primordial germ cell, oocyte, or sperm. The present invention is also directed to a method for making such a genetically-engineered DNA sequence, which method comprises joining a sequence encoding a BChE-hSA fusion protein with a urinary endothelium-specific promoter that directs expression of the BChE-hSA fusion protein and at least one signal sequence that provides secretion of the expressed BChE-hSA fusion protein.

The invention is also directed to a method for producing a transgenic mammal that upon lactation secretes a BChE-hSA fusion protein in its milk, which method comprises allowing an embryo, into which at least one genetically-engineered DNA sequence, comprising (i) a sequence encoding a BChE-hSA fusion protein, linked through a DNA linker that encodes at least 7 amino acid residues, preferably composed of 6 glycines and one serine in a sequential order; (ii) a mammary gland-specific promoter; and (iii) at least one signal sequence that provides secretion of the BChE-hSA fusion protein into the milk of the mammal, has been introduced, to grow when transferred into a recipient female mammal, resulting in the recipient female mammal giving birth to the transgenic mammal. In one embodiment, this method further comprises introducing the genetically-engineered DNA sequence into a cell of the embryo, or into a cell that will form at least part of the embryo. In specific embodiments, introducing the genetically-engineered DNA sequence comprises pronuclear or cytoplasmic microinjection of the DNA sequence; combining a mammalian cell stably transfected with the DNA sequence with a non-transgenic mammalian embryo; or introducing the DNA sequence into a non-human mammalian oocyte; and activating the oocyte to develop into an embryo.

The invention is further directed to a method for producing a transgenic mammal that upon lactation secretes a BChE-hSA fusion protein in its milk, which method comprises cloning or breeding of a transgenic mammal, the genome of which comprises a DNA sequence encoding a BChE enzyme and a hSA protein, linked through a DNA linker that encodes at least 7 amino acid residues, preferably composed of 6 glycines and one serine in a sequential order, operably linked to a mammary gland-specific promoter, wherein the sequence further comprises a signal sequence that provides secretion of the BChE-hSA fusion protein into the milk of the mammal.

The invention is also directed to a method for producing a transgenic mammal that secretes a BChE-hSA fusion protein in its urine, which method comprises allowing an embryo, into which at least one genetically-engineered DNA sequence, comprising (i) a sequence encoding a BChE-hSA fusion protein; (ii) a urinary endothelium-specific promoter; and (iii) at least one signal sequence that provides secretion of the BChE-hSA fusion protein into the urine of the mammal, has been introduced, to grow when transferred into a recipient female mammal, resulting in the recipient female mammal giving birth to the transgenic mammal. In one embodiment, this method further comprises introducing the genetically-engineered DNA sequence into a cell of the embryo, or into a cell that will form at least part of the embryo. In specific embodiments, introducing the genetically-engineered DNA sequence comprises pronuclear or cytoplasmic microinjection of the DNA sequence; combining a mammalian cell stably transfected with the DNA sequence with a non-transgenic mammalian embryo; or introducing the DNA sequence into a non-human mammalian oocyte; and activating the oocyte to develop into an embryo.

The invention is further directed to a method for producing a transgenic mammal that secretes a BChE-hSA fusion protein in its urine, which method comprises cloning or breeding of a transgenic mammal, the genome of which comprises a DNA sequence encoding a BChE-hSA fusion protein, operably linked to a urinary endothelium-specific promoter, wherein the sequence further comprises a signal sequence that provides secretion of the BChE-hSA fusion protein into the urine of the mammal.

The invention is directed to a method for producing a BChE-hSA fusion protein, which method comprises: (a) inducing or maintaining lactation of a transgenic mammal, the genome of which comprises a DNA sequence encoding a BChE-hSA fusion protein, linked through a DNA linker that encodes at least 7 amino acid residues, preferably composed of 6 glycines and one serine in a sequential order, operably linked to a mammary gland-specific promoter, where the sequence further comprises a signal sequence that provides secretion of the BChE enzyme into the milk of the mammal; and (b) extracting milk from the lactating mammal. In a specific embodiments, this method may comprise the additional steps of isolating the BChE-hSA fusion protein.

Accordingly, the invention is also directed to the milk of a non-human mammal comprising a human BChE-hSA fusion protein, and to milk comprising a BChE-hSA fusion protein produced by a transgenic mammal according to the methods of the invention.

The invention is also directed to a method for producing a BChE-hSA fusion protein, which method comprises, extracting urine from a transgenic mammal, the genome of which comprises a DNA sequence encoding a BChE-hSA fusion protein, operably linked to a urinary endothelium-specific promoter, where the sequence further comprises a signal sequence that provides secretion of the BChE enzyme into the urine of the mammal. In specific embodiments, this method may comprise the additional steps of isolating the BChE-hSA fusion protein, or isolating and purifying the BChE-hSA fusion protein.

Accordingly, the invention is also directed to the urine of a non-human mammal comprising a human BChE-hSA fusion protein, and to urine comprising a BChE-hSA fusion protein produced by a transgenic mammal according to the methods of the invention.

The invention is also direct to a method for producing a BChE-hSA fusion protein in a culture of MAC-T or BHK cells, which method comprises: (a) culturing said cells, into which a DNA sequence comprising (i) a DNA sequence encoding a BChE-hSA fusion protein, linked through a DNA linker that encodes at least 7 amino acid residues, preferably composed of 6 glycines and one serine in a sequential order, (ii) a promoter that provides expression of the encoded BChE-hSA fusion protein within said cells, and (iii) a signal sequence that provides secretion of the BChE-hSA fusion protein into the cell culture medium, has been introduced; (b) culturing the cells; and (c) collecting the cell culture medium of the cell culture. In specific embodiments, this method may comprise the additional steps of isolating the BChE-hSA fusion protein, or isolating and purifying the BChE-hSA fusion protein. Accordingly, the invention also encompasses cell culture medium comprising a BChE-hSA fusion protein produced by cultured MAC-T or BHK cells according to this method.

The invention also encompasses cell culture medium from a culture of mammalian cells, which medium comprises a BChE-hSA fusion protein.

The invention also provides a method for producing a pharmaceutical composition, which comprises combining a BChE-hSA fusion protein produced by a transgenic mammal or cultured MAC-T or BHK cells with a pharmaceutically acceptable carrier or excipient. Accordingly, the invention is further directed to methods for the treatment of organophosphate poisoning, post-surgical succinyl choline-induced apnea, and cocaine intoxication, which methods comprise administering to a subject in need thereof a therapeutically effective amount of a pharmaceutical composition produced by the methods of the invention.

The invention also encompasses a transgenic non-human mammal capable of expressing BChE-hSA fusion protein in both its milk and its urine. The genome of said transgenic mammal comprises (a) a DNA sequence encoding a BChE-hSA fusion protein, linked through a DNA linker that encodes at least 7 amino acid residues, preferably composed of 6 glycines and one serine in a sequential order, operably linked to a mammary gland-specific promoter, and further comprising a signal sequence that provides secretion of the BChE-hSA fusion protein into the milk of the mammal; and (b) a DNA sequence encoding a BChE-hSA fusion protein, linked through a DNA linker that encodes at least 7 amino acid residues, preferably composed of 6 glycines and one serine in a sequential order, operably linked to a urinary endothelium-specific promoter, and further comprising a signal sequence that provides secretion of the BChE-hSA fusion protein into the urine of the mammal. These DNA sequences may be encoded in a single, bi-cistronic expression construct, or in independent expression constructs.

In a further aspect, the present invention relates to a method for producing a recombinant enzymatically active butyrylcholinesterase (BChE) polypeptide fused to a polypeptide sequence that confers additional stability to said BChE when the latter is administered to an animal, such as a human being. Preferably, the BChE is fused to a serum albumin, especially human serum albumin (HSA). To facilitate the formation of native, active conformations of the BChE, and possibly the HSA, the BChE and fused polypeptide are separated by a linker, such as an amino acid sequence, preferaqbly one of at least about 7 amino acids. In accordance with the present invention, such a fusion protein is produced in vitro, such as in a cell culture, or is synthesized directly as a polypeptide sequence, or is prepared by chemically linking said BChE polypeptide to said fused polypeptide, such as HSA. Such fusion protein may also be prepared in vivo, using a transgenic animal, especially a mouse or goat, whereby the fusion protein is secreted into the milk or urine, or both, of said animal. In the latter case, the fusion protein is then prepared as a purified product using methods well known in the art for protein isolation and purification.

The present invention also relates to an isolated fusion protein, comprising an enzymatically active BChE enzyme and a mean residence time enhancing protein that increases the mean residence time of said fusion protein relative to said BChE when intravenously administered to a mammal. Preferably, the mean residence time enhancing protein is human serum albumin (hSA). Said protein will also commonly possess a signal sequence that directs secretion of the protein from a cell, such as for producing the fusion protein in the milk or urine of an animal. The linker sequence is available to promote independent folding and activity of said BChE as well as the hSA.

In another aspect, the present invention relates to an isolated polynucleotide encoding such fusion proteins. These polynucleotides may incorporate sequences that encode signal sequences as well as tissue-specific promoter sequences for directing expression of the fusion protein in milk or urine, especially in mice and goats.

In another aspect, the present invention relates to a non-human transgenic mammal that upon lactation, expresses in its milk a fusion protein of the invention.

In another aspect, the present invention relates to a non-human transgenic mammal as above, wherein the genome of the mammal comprises a polynucleotide of the invention. Such animals will commonly produce the fusion protein of the invention in their milk or urine.

Most preferred for such fusion protein is one in which the BChE is human BChE has an amino acid sequence as depicted in SEQ ID NO: 2.

In an additional aspect, the present invention relates to a method for producing a transgenic mammal that secretes a fusion protein of the invention, such as in its milk or urine. Preferably, this method further comprises introducing a polynucleotide of the invention into a cell of the embryo, or into a cell that will form at least part of the embryo, especially where introducing the genetically-engineered DNA sequence comprises pronuclear or cytoplasmic microinjection of the DNA sequence.

In one such embodiment, the method comprises introducing the polynucleotide of the invention by combining a mammalian cell stably transfected with the DNA sequence with a non-transgenic mammalian embryo. In another embodiment, the method comprises introducing the genetically-engineered DNA sequence comprises the steps of (a) introducing the DNA sequence into a non-human mammalian oocyte; and (b) activating the oocyte to develop into an embryo.

Such methods further comprise introducing the polynucleotide of the invention into a cell of the embryo, or into a cell that will form at least part of the embryo, including where the method comprises pronuclear or cytoplasmic microinjection of the polynucleotide.

The present invention also relates to methods of producing such fusion proteins by extraction from milk or urine of the transgenic animals of the invention as well as to the milk or urine produced.

The present invention also relates to a cell culture medium comprising a fusion protein, said fusion protein comprising a BChE enzyme and an hSA protein, produced by cultured MAC-T or BHK cells according to any of the methods of the invention.

In a further aspect, the present invention relates to a method for the treatment of organophosphate poisoning, which comprises administering to a subject in need thereof a therapeutically effective amount of a pharmaceutical composition comprising a fusion protein of the invention. Such methods include the treatment of post-surgical, succinyl choline-induced apnea, which comprises administering to a subject in need thereof a therapeutically effective amount of a pharmaceutical composition produced by the methods of the invention. The invention also includes a method for the treatment of cocaine intoxication, which comprises administering to a subject in need thereof a therapeutically effective amount of a pharmaceutical composition produced by the methods of the invention.

### Definitions

Unless expressly stated otherwise, each of the indicated terms has the following meaning:

The term “butyrylcholinesterase enzyme” or “BChE enzyme” means a polypeptide capable of hydrolizing acetylcholine and butyrylcholine, and whose catalytic activity is inhibited by the chemical inhibitor iso-OMPA. Preferred BChE enzymes to be produced by the present invention are mammalian BChE enzymes. Preferred mammalian BChE enzymes include human BChE enzymes. Most preferrably, the primary amino acid sequence of the BChE enzyme is subtantially identical to that of the native mature human BChE protein (for example, SEQ ID NO: 2). Such a BChE enzyme may be encoded by a nucleic acid sequence that is substantially identical to that of the native human BChE cDNA sequence (for example, SEQ ID NO: 1). The term “BChE enzyme” also encompasses pharmaceutically acceptable salts of such a polypeptide.

The term “BChE-hSA fusion protein” means a fused polypeptide capable of hydrolizing acetylcholine and butyrylcholine, and whose catalytic activity is inhibited by the chemical inhibitor iso-OMPA. Preferred BChE-hSA fusion protein to be produced by the present invention are mammalian BChE-hSA fusion proteins. Preferred mammalian BChE-hSA fusion proteins include human BChE-hSA fusion proteins. Most preferably, the primary amino acid sequence of the BChE-hSA fusion protein is substantially identical to that of the native mature human BChE protein (for example, SEQ ID NO: 2) and that of the native mature human serum albumin, linked through an oligopolypeptide, preferably composed of at least 7 amino acid residues including 6 glycines and one serine in a sequential order (for example, SEQ ID NO: 50). Such a BChE-hSA fusion protein may be encoded by two nucleic acid sequences that are substantially identical to that of the native human BChE cDNA sequence (for example, SEQ ID NO: 1) and that of the native human serum albumin cDNA sequence, linked through a DNA linker that encodes at least 7 amino acid residues, preferably composed of 6 glycines and one serine in a sequential order (for example, SEQ ID NO: 49). The term “BChE-hSA fusion protein” also encompasses pharmaceutically acceptable salts of such a fused polypeptide.

The term “substantially identical” means a polypeptide or nucleic acid exhibiting at least 75%, preferably at least 85%, more preferably at least 90%, and most preferably at least 95% identity in comparison to a reference amino acid or nucleic acid sequence. For polypeptides, the length of sequence comparison will generally be at least 20 amino acids, preferably at least 30 amino acids, more preferably at least 40 amino acids, and most preferably at least 50 amino acids. For nucleic acids, the length of sequence comparison will generally be at least 60 nucleotides, preferably at least 90 nucleotides, and more preferably at least 120 nucleotides.

The term “recombinant butyrylcholinesterase” or “recombinant BChE” means a BChE enzyme produced by a transiently transfected, stably transfected, or transgenic host cell or animal as directed by one of the expression constructs of the invention. The term “recombinant BChE” also encompasses pharmaceutically acceptable salts of such a polypeptide.

The term “recombinant BChE-hSA fusion protein” means a fused polypeptide produced by a transiently transfected, stably transfected, or transgenic host cell or animal as directed by one of the expression constructs of the invention. The term “recombinant BChE-hSA fusion protein” also encompasses pharmaceutically acceptable salts of such a polypeptide.

The term “genetically-engineered DNA sequence” means a DNA sequence wherein the component sequence elements of the DNA sequence are organized within the DNA sequence in a manner not found in nature. Such a genetically-engineered DNA sequence may be found, for example, ex vivo as isolated DNA, in vivo as extra-chromosomal DNA, or in vivo as part of the genomic DNA.

The term “expression construct” or “construct” means a nucleic acid sequence comprising a target nucleic acid sequence or sequences whose expression is desired, operably linked to sequence elements which provide for the proper transcription and translation of the target nucleic acid sequence(s) within the chosen host cells. Such sequence elements may include a promoter, a signal sequence for secretion, a polyadenylation signal, intronic sequences, insulator sequences, and other elements described in the invention. The “expression construct” or “construct” may further comprise “vector sequences”. The term “vector sequences” means any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

The term “bi-cistronic construct” means any construct that provides for the expression of two independent translated products. These two products may be translated from a single mRNA encoded by the bi-cistronic construct or from two independent mRNAs where each of the mRNAs is encoded within the same bi-cistronic construct. The term “poly-cistronic construct” means any construct that provides for the expression of more than two independent translated products.

The term “operably linked” means that a target nucleic acid sequence and one or more regulatory sequences (e.g., promoters) are physically linked so as to permit expression of the polypeptide encoded by the target nucleic acid sequence within a host cell.

The term “signal sequence” means a nucleic acid sequence which, when incorporated into a nucleic acid sequence encoding a polypeptide, directs secretion of the translated polypeptide (e.g., a BChE enzyme and/or a BChE-hSA fusion protein and/or a glycosyltransferase) from cells which express said polypeptide. The signal sequence is preferably located at the 5′ end of the nucleic acid sequence encoding the polypetide, such that the polypeptide sequence encoded by the signal sequence is located at the N-terminus of the translated polypeptide. The term “signal peptide” means the peptide sequence resulting from translation of a signal sequence.

The term “mammary gland-specific promoter” means a promoter that drives expression of a polypedtide encoded by a nucleic acid sequence to which the promoter is operably linked, where said expression occurs primarily in the mammary cells of the mammal, wherefrom the expressed polypeptide may be secreted into the milk. Preferred mammary gland-specific promoters include the β-casein promoter and the whey acidic protein (WAP) promoter

The term “urinary endothelium-specific promoter” means a promoter that drives expression of a polypedtide encoded by a nucleic acid sequence to which the promoter is operably linked, where said expression occurs primarily in the endothelial cells of the kidney, ureter, bladder, and/or urethra, wherefrom the expressed polypeptide may be secreted into the urine. The term “urothelium” or “urothelial cells” refers to the endothelial cells forming the epithelial lining of the ureter, bladder, and urethra.

The term “host cell” means a cell which has been transfected with one or more expression constructs of the invention. Such host cells include mammalian cells in in vitro culture and cells found in vivo in an animal. Preferred in vitro cultured mammalian host cells include MAC-T cells and BHK cells.

The term “transfection” means the process of introducing one or more of the expression constructs of the invention into a host cell by any of the methods well established in the art, including (but not limited to) microinjection, electroporation, liposome-mediated transfection, calcium phosphate-mediated transfection, or virus-mediated transfection. A host cell into which an expression construct of the invention has been introduced by transfection is “transfected”. The term “transiently transfected cell” means a host cell wherein the introduced expression construct is not permanently integrated into the genome of the host cell or its progeny, and therefore may be eliminated from the host cell or its progeny over time. The term “stably transfected cell” means a host cell wherein the introduced expression construct has integrated into the genome of the host cell and its progeny.

The term “transgene” means any segment of an expression construct of the invention which has become integrated into the genome of a transfected host cell. Host cells containing such transgenes are “transgenic”. Animals composed partially or entirely of such transgenic host cells are “transgenic animals”. Preferably, the transgenic animals are transgenic mammals (e.g., rodents or ruminants). Animals composed partially, but not entirely, of such transgenic host cells are “chimeras” or “chimeric animals”.

### DESCRIPTION OF THE DRAWINGS

FIGS. 1A and 1B depict the cDNA and translated amino acid sequence of wild-type human BChE. The signal sequence is in bold. The signal peptide, which is cleaved during processing to produce the mature BChE protein, is underlined. Amino acids are represented by the standard one-letter code. * indicates the STOP codon.

FIG. 2 depicts the locations of altered residues in some naturally occuring human BChE variants (See also Table 1). Amino acids are represented by the standard one-letter code. One letter codes shown above the amino acid sequence represent the type of variant as follows: A=atypical; F=fluoride resistant; H, J, and K=H, J, and K variants; N=unstable variant; and S=Silent (no or very low activity) variants. Asterisks (*) shown below the amino acid sequence mark the residues of the catalytic triad.

FIG. 3 depicts a non-reducing BChE-activity gel of condition serum-free cell culture media from stably transfected cell lines expressing recombinant BChE. Conditioned, serum free media was from: Lane 1) MAC-T cells, untransfected control; Lane 2) MAC-T cells stably transfected with pCMV/IgKBChE; Lane 3) MAC-T cells stably transfected with pCMV/BChE-hSA; Lane 4) BHK cells, untransfected control; Lane 5) BHK cells stably transfected with pCMV/BChE-hSA. Lane 6) was purified human serum BChE, positive control.

FIG. 4 is a schematic depicting the generation of the pBCNN/BChE expression construct. SS=signal sequence. This expression construct provides for expression of recombinant BChE in the mammary gland of a transgenic mammal, and for the secretion of the recombinant BChE into the milk of a lactating transgenic mammal.

FIG. 5 is a schematic depicting the exons and introns of the goat β-casein locus that are contained in the NotI linearized fragment of pBCNN/BChE. This BCNN-BChE fragment contains a BChE encoding sequence in place of goat β-casein locus sequences from the end of exon 2 through the majority of exon 7.

FIG. 6 depicts a non-reducing BChE-activity gel of the whey phase of milk collected from BCNN-BChE transgenic mice. Whey phase samples were as follows: Lane 1) milk collected from BCNN-BChE transgenic mice; and Lanes 2 and 3) milk collected from non-trangenic mice (negative control) rBChE=recombinant BChE.

FIG. 7 depicts a non-reducing BChE-activity gel of the whey phase of milk collected from BCNN-BChE transgenic goats. Whey phase samples were as follows: Lane 1) purified human serum BChE, positive control; Lane 2) milk from a non-transgenic goat, negative control; and Lanes 3-5) three independent milk samples collected from the same female transgenic goat.

FIG. 8 depicts silver staining of a denaturing SDS-PAGE gel of recombinant BChE purified from milk collected from a BCNN-BChE transgenic goat. Samples were reduced in the presence of DTT prior to loading onto the gel. Samples were as follows: Lane 1) 0.2 μg of BChE purified from the milk of a BCNN-BChE transgenic goat; and Lane 2) 0.2 μg of purified human serum BChE, positive control.

FIG. 9 is a schematic depicting the generation of the pWAP/BChE construct. This expression construct provides for expression of recombinant BChE in the mammary gland of a transgenic mammal, and for the secretion of the recombinant BChE into the milk of a lactating transgenic mammal

FIG. 10 is a shematic depicting the linear NotI fragment of pWAP/BChE.

FIG. 11 is a schematic depicting the strategy for generating the expression construct pUM/BChE. UM=uromodulin. SS=signal sequence. This expression construct will provide for expression of recombinant BChE in the kidney of a transgenic mammal, and for the secretion of the recombinant BChE into the urine of a transgenic mammal.

FIG. 12 is a schematic depicting the strategy for generating the expression construct pUP11/BChE. UPII=uroplakin II. SS=signal sequence. This expression construct will provide for expression of recombinant BChE in the urothelium of a transgenic mammal, and for the secretion of the recombinant BChE into the urine of a transgenic mammal.

FIGS. 13 depicts the cDNA and translated amino acid sequence of the BChE-hSA. The signal sequence is in bold. The signal peptide, which is cleaved during processing to produce the mature BChE protein, is underlined. Amino acids are represented by the standard one-letter code. The DNA linker between BChE and hSA is italic and in bold whereas the oligopeptide linker between BChE and hSA is italic and underlined. Nine single-nucleotide-mutations (boxed) were found in the hSA cDNA when compared with a published hSA sequence (GenBank accession No #V00495). However, only one mutation at nucleotide 2977 (a→g, boxed) causes a change of an amino acid residue (K→E, in bold and boxed). * indicates the STOP codon.

FIG. 14 demonstrates BChE activity of the BChE-hSA fusion protein produced in vitro and in vivo. Samples (15 μl) were loaded on a 4-20% Tris-glycine non-denaturing gel in the following order: lane 1, purified plasma huBChE (10 U/ml, courtesy of Dr. O. Lockridge); lane 2, Harvest 8 of conditioned media from the hollow fiber system (10 U/ml); lane 3, control conditioned media; lane 4, diluted milk sample from a F1 transgenic mouse (10 u/ml); lane 5, diluted milk sample from a F2 transgenic mouse (10 u/ml); lane 6, diluted milk sample from a non-transgenic FVB mouse (1:30); lane 7, diluted milk sample from a transgenic goat (10 u/ml); lane 8, diluted milk samples from another transgenic goat (10 u/ml); lane 9, purified BChE-hSA sample from the milk of the same transgenic goat as shown in lane 8; lane 10, diluted milk sample from a non-transgenic goat (1:30).

FIG. 15 depicts Western blot analysis under denaturing and reducing conditions. Immunodetection was performed with a polyclonal anti-huBChE antibody from rabbit (Dako, 1: 1000) and a secondary HRP-conjugated anti-rabbit AB (Promega, 1:5000). 15 μl samples were loaded in the following order: lane 1, Biotinylated molecular markers (Molecular Probes); lane 2, purified plasma huBChE (10 U/ml, courtesy of Dr. O. Lockridge); lane 3, Harvest 8 of conditioned media from the hollow fiber system (10 U/ml); lane 4, control conditioned media; lane 5, diluted milk sample from a F1 transgenic mouse (307-1A7F, 10 u/ml); lane 6, diluted milk sample from a non-transgenic FVB mouse (1:30); lane 7, diluted milk sample from a transgenic goat (2237, BBLA goat, 10 u/ml); lane 8, diluted milk sample from another transgenic goat (2177, 10 u/ml); lane 9, purified BChE-hSA sample from the milk of the same transgenic goat as shown in lane 8; lane 10, diluted milk sample from a non-transgenic goat (1:30).

FIG. 16 depicts Silver-stained SDS-PAGE. Samples were loaded onto a pre-cast 4-20% Tris-Glycine polyacrylamide gel under denaturing and reducing conditions in the following order: lane 1, molecular weight marker (Bio-Rad); lane 2, purified BChE-hSA from milk of the transgenic goat, 2177.

FIG. 17 depicts the in vivo clearance of the BChE-hSA fusion protein produced in vitro (A) and a transgenically produced BChE preparation containing >70% tetramer (B) in juvenile pigs. The pigs were i.v. injected with the proteins and residual BChE activity (%) was plotted versus time (hours) for each animal.

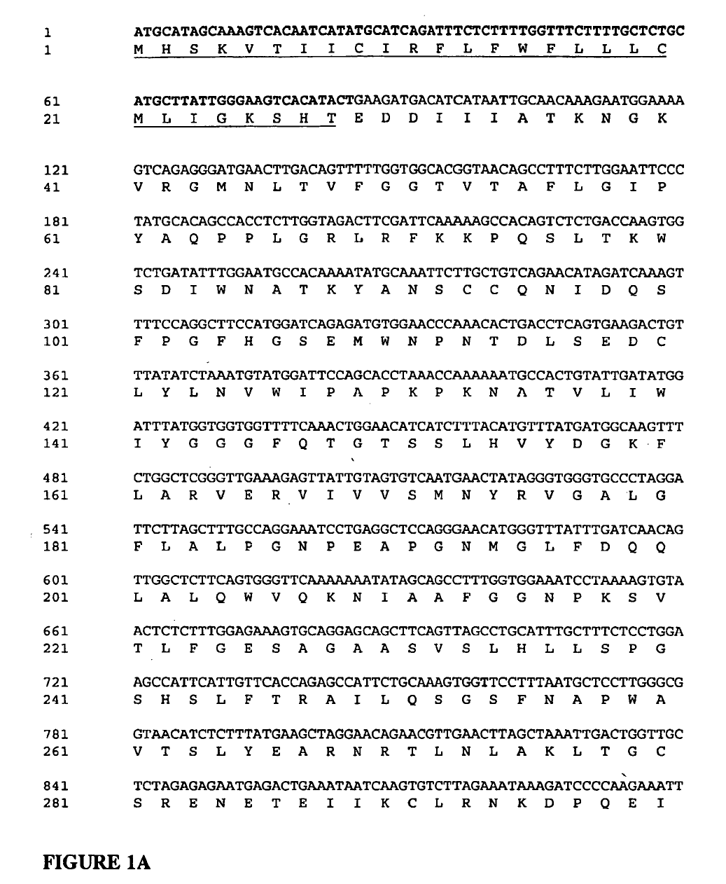

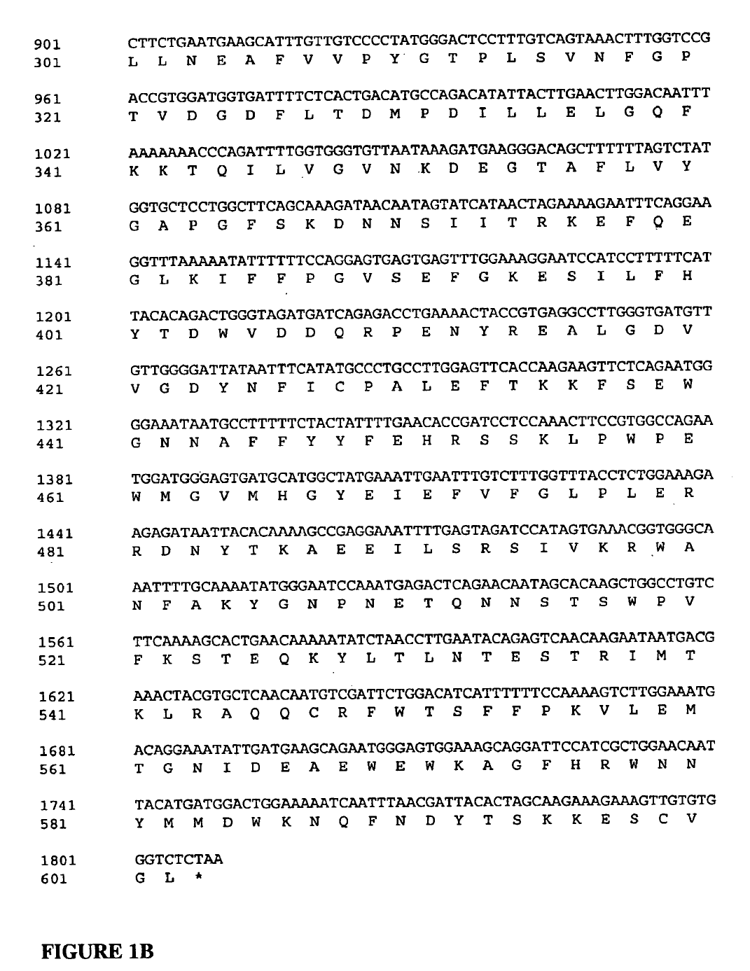

### DETAILED DESCRIPTION OF THE INVENTION

Selection of BChE Enzymes

Butyrylcholinesterase derived from human serum is a globular, tetrameric molecule with a molecular mass of approximately 340 kDa. Nine Asn-linked carbohydrate chains are found on each 574-amino acid subunit. The tetrameric form of BChE is the most stable and is preferred for therapeutic purposes. Wildtype, variant, and artificial BChE enzymes can be produced by transgenic mammals according to the invention. BChE enzymes produced according to the instant invention have the ability to bind and/or hydrolyze organophosphate pesticides, war gases, succinylcholine, or cocaine.

Preferably, the BChE enzyme produced according to the invention comprises an amino acid sequence that is substantially identical to a sequence found in a mammalian BChE, more preferably, human BChE, and may be produced as a tetramer, a trimer, a dimer, or a monomer. In a preferred embodiment, the BChE of the invention has a glycosylation profile that is substantially similar to that of native human BChE.

Fusion of BChE to hSA

In another preferred embodiment, the BChE enzyme produced according to the invention is fused to a human serum albumin (hSA) moiety. This fusion to hSA is expected to exhibit high plasma stability, and is expected to be either weakly or non-immunogenic for the organism in which it is used.

The BChE produced according to the present invention is preferably in tetrameric form. It is believed that the tetrameric form of BChE is more stable and has a longer half-life in the plasma, thereby increasing its therapeutic effectiveness. BChE expressed recombinantly in CHO (Chinese hamster ovary) cells was found not to be in the more stable tetrameric form, but rather consisted of approximately 55% dimers, 10-30% tetramers and 15-40% monomers [Blong, et al. Biochem. J. (1997) 327:747-757]. Recent studies have shown that a proline-rich amino acid sequence from the N-terminus of the collagen-tail protein caused acetylcholinesterase to assemble into the tetrameric form [Bon, et al. J. Biol. Chem. (1997) 272(5):3016-3021 and Krejci, et al. J. Biol. Chem. (1997) 272:22840-22847]. Thus, to increase the amount of tetrameric BChE enzyme formed according to the invention, the DNA sequence encoding the BChE enzyme of the invention may comprise a proline-rich attachment domain (PRAD), which recruits recombinant BChE subunits (e.g., monomers, dimers and trimers) to form tetrameric associations. The PRAD preferably comprises at least six amino acid residues followed by a string of at least 10 proline residues. An example of a PRAD useful in the invention comprises the sequence (Glu-Ser-Thr-Gly₃-Pro₁₀) (SEQ ID NO: 40). The PRAD may be included in a bi-cistronic expression construct which encodes both the PRAD and the BChE enzyme, or the PRAD and the BChE enzyme may be encoded in separate constructs. Alternatively, encoded PRAD may be attached directed to the encoded BChE enzyme. The invention also contemplates addition of a PRAD, which can be synthetic (e.g., polyproline) or naturally occurring, to a mixture comprising recombinant BChE, to induce rearrangement of the BChE enzyme into tetramers.

Although it is believed that tetrameric BChE will be the most therapeutically effective form of BChE for the treatment and/or prevention of organophosphate poisoning, other forms of the enzyme (e.g., monomers, dimers and trimers) have demonstrated substrate activity and are also encompassed by the invention. However, the observation that non-tetrameric forms of BChE are less stable in vivo does not rule out their usefulness in in vivo applications. Higher doses or more frequent in vivo administration of the non-tetrameric forms of BChE can result in satisfactory therapeutic activity.

The non-tetrameric forms of BChE are also useful in applications which do not require in vivo administration, such as the clean-up of lands used to store organophosphate compounds, as well as decontamination of military equipment exposed to organophosphates. For ex vivo use, these non-tetrameric forms of BChE may be incorporated into sponges, sprays, cleaning solutions or other materials useful for the topical application of the enzyme to equipment and personnel. These forms of the enzyme may also be applied externally to the skin and clothes of human patients who have been exposed to organophosphate compounds. The non-tetrameric forms of the enzyme may also find applications as barriers and sealants applied to the seams and closures of military clothing and gas masks used in chemical warfare situations.

Fusion of BChE to Human Serum Albumin

BChE has been shown to be an effective treatment against multiple LD50s of organophosphates. A prerequisite for such use of BChE is a prolonged circulatory half-life. A means of achieving plasma stability and longer half-life of recombinant BChE produced according to the invention is to provide a recombinantly produced BChE fused to hSA. This fusion protein is believed to exhibit high plasma stability and an advantageous distribution in the body, and is expected to be either weakly or non-immunogenic for the organism in which it is used.

The BChE enzyme amino acid sequences and the hSA amino acid sequences of the fusion protein may or may not be separated by linker amino acid sequences (e.g., a poly-glycine linker). Such linker amino acid sequences are often included to promote proper folding of the different domains of a fusion protein (e.g., hSA domain and BChE enzyme domain). By promoting proper folding of the BChE enzyme domain, such linker sequences may promote maintenace of catalytic activity.

For example, hSA may be fused to either the N-terminus or the C-terminus of BChE. In preferred embosiments, the hSA moiety is fused to the C-terminal end of the BChE enzyme. This fusion is expected to provide a fusion protein that maintains BChE catalytic activity. In one embodiment for fusion of hSA to the N-terminal end of BChE, the plasmid pYG404 can be used, as described in EP 361,991. This plasmid contains a restriction fragment encoding the prepro-hSA gene. The BChE-encoding nucleic acid sequence can be amplified by PCR using primers that are exclusive of the termination codon and signal sequence. This BChE-encoding PCR product may be introduced at the 3′ end of the pYG404 prepro-hSA sequence, in the same translational frame. In one embodiment for fusion of hSA to the C-terminal end of BChE, the hSA-encoding nucleic acid sequence, without its signal sequence, is fused in translational frame to the 3′ end of the BChE-encoding nucleic acid sequence.

In another embodiment, purified recombinant BChE may be conjugated in vitro to a hSA polypeptide. Conjugation may be achieved by any appropriate chemical or affinity ligand method. Particularly useful are hSA and BChE polypeptides with monovalent affinity ligand modifications. For in vitro conjugation, each protein to be conjugated (e.g. hSA) can be separately produced by recombinant methods and isolated to the necessary purity, followed by in vitro conjugation, prior to administration.

In accordance with the foregoing, the present invention relates to a method for producing a fusion protein that comprises an enzymatically active butyrylcholinesterase (BChE) enzyme and a human serum albumin (hSA) (which increases the mean residence time of said fusion protein relative to said BChE when intravenously administered to a mammal, comprising expressing said fusion protein in a recombinant cell that comprises a polynucleotide encoding said fusion protein. In specific embodiments, such mammal is a swine, a mouse or a human being.

In a preferred embodiment, the BChE and the hSA are separated by an oligopeptide linker that promotes independent folding and activity of said BChE. Preferably, said oligopeptide linker is at least 7 amino acids in length, such as where the oligopeptide linker is preferably composed of glycine and/or serine residues. In other preferred embodiments, the cell is a BHK cell. Also preferred is where the BChE is human BChE. Preferably, the polynucleotide further comprises a signal sequence that directs secretion of said fusion protein by the cell. Also preferred is where the BChE enzyme comprises the amino acid sequence of SEQ ID NO: 2 and the polynucleotide comprises the nucleotide sequence of SEQ ID NO: 1. Commonly, the cell will also comprise a polynucleotide sequence that encodes a glycosyltransferase, said polynucleotide being operably linked to a promoter, so that following expression said glycosyltransferase will glycosylate the BChE portion of the fusion protein. In one embodiment, the polynucleotide encoding the fusion protein is part of a plasmid.

The present invention is also drawn to an isolated fusion protein, comprising an enzymatically active BChE enzyme and an hSA and where the fusion protein further comprises a linker located between said BChE enzyme and said hSA protein and wherein said linker promotes independent folding and activity of said BChE, and comprises an amino acid sequence, preferably at least 7 amino acid residues in length, most preferably glycine and serine residues. Also preferred is where said BChE is a human BChE and said hSA is a human serum albumin, such as the amino acid sequence of SEQ ID NO: 50 and further comprising a signal sequence that directs secretion of said fusion protein from a cell and wherein said signal sequence directs secretion into milk or into urine.

The present invention is further drawn to an isolated polynucleotide, comprising: (i) a nucleotide sequence encoding the BChE-hSA fusion protein, where the fusion protein further comprises a linker located between said BChE enzyme and said hSA protein and wherein said linker promotes independent folding and activity of said BChE, and comprises an amino acid sequence, preferably at least 7 amino acid residues in length, most preferably glycine and serine residues. (ii) a promoter that directs expression of the fusion protein, and (iii) at least one signal sequence that provides secretion of the expressed fusion protein from a cell, preferably wherein said amino acid sequence comprises at least 6 amino acid residues. Preferably, the BChE is a human BChE and said hSA is a human serum albumin, such as the amino acid sequence of SEQ ID NO: 50 and/or wherein the polynucleotide comprises the nucleotide sequence of SEQ ID NO: 49.

In preferred embodiments, the signal sequence directs secretion into milk or into urine. In preferred embodiments thereof, the promoter is a mammary gland-specific promoter selected from the group consisting of a WAP (whey acidic protein) promoter and a casein promoter (for secretion into milk) or the promoter is a urinary endothelium-specific promoter selected from the group consisting of a uroplakin promoter or a uromodulin promoter (for secretion into urine).

The present invention also relates to a recombinant cell that comprises the isolated polynucleotide of the invention, such as where the the cell is a MAC-T (mammary epithelial) cell or a BHK (baby hamster kidney) cell. In other embodiments, the cell is selected from the group of embryonic stem cells, embryonal carcinoma cells, primordial germ cells, oocytes, or sperm.

The present invention also encompasses a non-human mammalian embryo that comprises a polynucleotide of the invention.

The present invention is also drawn to a non-human transgenic mammal that upon lactation, expresses in its milk or urine the fusion protein of the invention, preferably wherein the BChE is fused to hSA. The mammal is preferably a mouse or a goat. The genome of said non-human transgenic mammal preferably comprises a polynucleotide of the invention. In preferred embodiments of such mammal the promoter is a mammary gland-specific promoter selected from the group consisting of a WAP (whey acidic protein) promoter and a casein promoter (for secretion into milk) or is a urinary endothelium-specific promoter selected from the group consisting of a uroplakin promoter or a uromodulin promoter.

In other preferred embodiments of such mammal, the genome of the mammal further comprises a DNA sequence encoding a glycosyltransferase, operably linked to a mammary gland-specific promoter, and a signal sequence that provides secretion of the glycosyltransferase. Preferred is where the mammary gland-specific promoter is a casein promoter or a whey acidic protein (WAP) promoter. Also preferred is where the genome of the mammal further comprises a DNA sequence encoding a glycosyltransferase, operably linked to a urinary gland-specific promoter, and a signal sequence that provides secretion of the glycosyltransferase. Preferably, the promoter is a urinary endothelium-specific promoter selected from the group consisting of a uroplakin promoter or a uromodulin promoter. Preferably, said mammal is a mouse or a goat.

The present invention is also drawn to a method for producing a transgenic mammal that upon lactation secretes the fusion protein of claim 16 in its milk, which method comprises allowing an embryo, into which at least one genetically-engineered DNA sequence, comprising (i) a sequence encoding said fusion protein; (ii) a mammary gland-specific promoter; and (iii) a signal sequence that provides secretion of the fusion protein into the milk of the mammal, has been introduced, to grow when transferred into a recipient female mammal, resulting in the recipient female mammal giving birth to the transgenic mammal.

Preferably, such method further comprises introducing the genetically-engineered DNA sequence into a cell of the embryo, or into a cell that will form at least part of the embryo. A preferred such procedure is wherein introducing the genetically-engineered DNA sequence comprises pronuclear or cytoplasmic microinjection of the DNA sequence or wherein introducing the genetically-engineered DNA sequence comprises combining a mammalian cell stably transfected with the DNA sequence with a non-transgenic mammalian embryo. Also preferred is the method wherein introducing the genetically-engineered DNA sequence comprises the steps of (a) introducing the DNA sequence into a non-human mammalian oocyte; and (b) activating the oocyte to develop into an embryo.

In accordance with the foregoing, the present invention also relates to a method for producing a transgenic mammal that upon lactation secretes a fusion protein of the invention, in its milk or urine, which method comprises cloning or breeding of a transgenic mammal, the genome of which comprises a DNA sequence encoding said fusion protein, operably linked to a mammary gland-specific promoter or a urinary specific promoter, wherein the sequence further comprises a signal sequence that provides secretion of the fusion protein into the milk or urine of the mammal.

The present invention further relates to a method for producing a fusion protein of the invention, comprising: (a) inducing or maintaining lactation or urination of a transgenic mammal, the genome of which comprises a DNA sequence encoding said fusion protein, operably linked to a mammary gland-specific promoter or urinary specific promoter, wherein the sequence further comprises a signal sequence that provides secretion of the fusion protein into the milk or urine of the mammal; and (b) extracting milk from the lactating mammal or urine from the urinating mammal. In a preferred embodiment thereof, such method comprises the additional step of isolating the fusion protein from the extracted milk or urine. In further preferred steps, the method comprises purifying the fusion protein. The present invention also encompasses the milk or urine of said non-human mammal comprising the fusion protein of the invention, including where the milk is whole milk or is defatted milk.

The present invention further relates to a composition comprising a fusion protein of the invention, preferably where the BChE is linked to hSA, in a pharmaceutically acceptable carrier.

The present invention also encompasses a method for treating organophosphate poisoning, comprising administering to a subject in need thereof a therapeutically effective amount of a pharmaceutical composition of the invention. The present invention further relates to a method for the treatment of post-surgical, succinyl choline-induced apnea, which comprises administering to a subject in need thereof a therapeutically effective amount of a pharmaceutical composition of the invention. The present invention further relates to a method for the treatment of cocaine intoxication, which comprises administering to a subject in need thereof a therapeutically effective amount of a pharmaceutical composition of the invention.

BChE-hSA Fusion Protein Glycosylation Profile

Naturally occurring human serum BChE and hSA are highly glycosylated. For example, the carbohydrate content of cholinesterases, including human BChE, generally comprises about 33-40% N-acetylglucosamine, 21-31% mannose, 18-21% galactose, and 15-18% sialic acid. It has been suggested that the relatively high stability of the globular tetrameric form of human plasma BChE may be associated with the capping of the terminal carbohydrate residues with sialic acid.

Mammalian cells used in recombinant protein synthesis have glycosylation capabilities, but if BChE is not normally expressed by these host cells, the glycosylation pattern of the recombinantly produced BChE may differ from that of the natural glycoprotein. Since BChE is a heavily glycosylated molecule, it is difficult for a recombinant host cell to modify it faithfully. Indeed, it has been shown that BChE produced in CHO cells had a lower sugar content than that found in the native human protein [Yuan, et al. Acta Pharmacologica Sinica, (1999), 20:74-80].

As a means of producing recombinant BChE-hSA fusion protein with a glycosylation profile that more closely resembles that of the native enzyme, the present invention is directed to transgenic animals that express both a BChE-hSA fusion protein and one or more glycosyltransferases in their mammary glands and/or urinary endothelium, as well as cultured mammalian cells that express both a BChE-hSA fusion protein and one or more glycosyltransferases. The presence of the glycosyltransferases in the intracellular secretory pathway of cells that are also expressing a secreted form of BChE-hSA fusion protein catalyzes the transfer of glycan moieties to said BChE-hSA fusion proteins. The invention also encompasses addition of one or more glycosyltransferases to an in vitro reaction for the transfer of glycan moieties to a recombinant BChE-hSA fusion protein produced by the transgenic animals or transfected mammalian cell lines of the invention. For example, recombinant BChE-hSA fusion protein may be sialylated using the in vitro reaction conditions described in Chitlaru, et al. Biochem. J. (1998) 336:647-658. Thus, the glycosyltransferase which catalyzes transfer of glycans to the BChE-hSA fusion protein may be expressed by the same cell that expresses the fusion protein, or the glycosyltransferase may be obtained from an external source and added to the recombinant BChE-hSA fusion protein.

Most bioactive terminal sugars are attached to common core structures by “terminal” glycosyltransferases. When two terminal enzymes compete with each other, the ultimate carbohydrate structure is determined by the specificity of the enzyme that acts first. According to the present invention, a terminal or branching glycosyltransferase, which is not normally produced by the host cell, is introduced and “over-expressed” in the cell according to the methods described herein. The recombinantly produced glycosyltransferase will successfully compete with the endogenous enzymes, producing a recombinant BChE-hSA fusion protein which has a glycosylation profile which more closely resembles that of the native enzymes. The methods of the invention alter the glycosylation capabilities of mammary, bladder, or kidney epithelial cells in order to control carbohydrate attachment on the secreted BChE-hSA fusion protein. Carbohydrate moieties are commonly attached to asparagine, serine, or threonine residues.

The basic procedure involves introduction of an expression construct comprising a nucleic acid sequence encoding a glycosyltransferase enzyme operably linked to elements that allow expression of the glycosyltransferase enzyme in the tissue of interest. A second expression construct, one of the BChE-hSA fusion protein-encoding expression constructs described herein, is also introduced. Alternatively, the BChE-hSA fusion protein and the glycosyltransferase may be encoded in a single bi-cistronic construct. An example of a bi-cistronic construct of the invention would be a construct which comprises a WAP promoter; a nucleic acid sequence which encodes both a BChE-hSA fusion protein and a glycosyltransferase, in which an IRES (internal ribosomal entry site) is included between the sequence encoding the BChE-hSA fusion protein and the sequence encoding the glycosyltransferase; and signal sequences to provide secretion of the BChE-hSA fusion protein and the glycosyltransferase. This construct may be introduced into the genome of a mammalian host cell by techniques well known in the art including microinjection, electroporation, and liposome-mediated transfection, calcium phosphate-mediated transfection, virus-mediated transfection, and nuclear transfer techniques. Accordingly, the recombinant BChE-hSA fusion protein that is ultimately secreted by the host cell will have a more predictable glycosylation pattern. The invention also encompasses the generation of transgenic mammals that secrete a BChE-hSA fusion protein and a glycosyltransferase in their milk and/or urine through cross-breeding of transgenic mammals that secrete a BChE-hSA fusion protein only with transgenic mammals of the same species that secrete the desired glycosyltransferases, to produce transgenic mammals that secrete these enzymes.

The preferred glycosyltransferase enzymes for use in accordance with the present invention are sialyltransferases. Other enzymes that alter the glycosylation machinery whose production within a host cell may be desirable include fucosyltransferases, mannosyltransferases, acetylases, glucoronyltransferases, glucosylepimerases, galactosyltransferases, β-acetylgalactosaminyltransferases, N-acetylglucosaminyltransferases, and sulfotransferases. For a description of such transferases see, for example; Hennet. Cell Mol. Life Sci. (2002) 59:1081-1095; Harduin-Lepers, et al. Biochimie (2001) 83:727-737; and Takashima, et al. J. Biol. Chem. (2002) 277:45719-45728. Please refer to Sequences that encode any one or more of such glycosyltransferases may be introduced into host cells according to the invention. These glycosyltransferases may be encoded in separate expression constructs, or included in any one or more bi-cistronic or poly-cistronic constructs. Thus, it should be noted that the invention allows for simultaneous expression in the milk and/or urine of a mammal of a BChE-hSA fusion protein and one or more glycsoyltransferases. The glycosyltransferases to be expressed are selected so as to effect transfer of one or more of the desired carbohydrate moieties to the BChE-hSA fusion protein.

In the event that independent transcripts to encode the BChE-hSA fusion protein and the respective glycosyltransferses, it is preferred that different promoters are used to express the different transcripts. For example, if the nucleic acid sequence encoding the BChE-hSA fusion protein is operably linked to a mammary gland-specific casein promoter, it is preferred that nucleic acid sequence encoding the glycosyltransferase is operably linked to a different mammary gland-specific promoter, such as a WAP promoter. Although it is preferred to use different promoters in this instance, the invention also encompasses use of the same promoter.

In accordance with the foregoing, the present invention relates to a method for producing a fusion protein that comprises an enzymatically active butyrylcholinesterase (BChE) enzyme and a human serum albumin (hSA) (which increases the mean residence time of said fusion protein relative to said BChE when intravenously administered to a mammal), comprising expressing said fusion protein in a recombinant cell that comprises a polynucleotide encoding said fusion protein. In specific embodiments, such mammal is a swine, a mouse or a human being.

In a preferred embodiment, the BChE and the hSA are separated by an oligopeptide linker that promotes independent folding and activity of said BChE. Preferably, said oligopeptide linker is at least 6 amino acids in length, such as where the oligopeptide linker is preferably composed of glycine residues. In other preferred embodiments, the cell is a BHK cell. Also preferred is where the BChE is human BChE. Preferably, the polynucleotide further comprises a signal sequence that directs secretion of said fusion protein by the cell. Also preferred is where the BChE enzyme comprises the amino acid sequence of SEQ ID NO: 2 and the polynucleotide comprises the nucleotide sequence of SEQ ID NO: 1. Commonly, the cell will also comprise a polynucleotide sequence that encodes a glycosyltransferase, said polynucleotide being operably linked to a promoter, so that following expression said glycosyltransferase will glycosylate the BChE portion of the fusion protein. In one embodiment, the polynucleotide encoding the fusion protein is part of a plasmid.

The present invention is also drawn to an isolated fusion protein, comprising an enzymatically active BChE enzyme and an hSA and where the fusion protein further comprises a linker located between said BChE enzyme and said hSA protein and wherein said linker promotes independent folding and activity of said BChE, including a linker comprising an amino acid sequence, preferably at least 6 amino acid residues in length, most preferably glycine residues. Also preferred is where said BChE is a human BChE, such as the amino acid sequence of SEQ ID NO: 2 and further comprising a signal sequence that directs secretion of said fusion protein from a cell and wherein said signal sequence directs secretion into milk or into urine.

The present invention is further drawn to an isolated polynucleotide, comprising: (i) a nucleotide sequence encoding the fusion protein of claim 16, (ii) a promoter that directs expression of the fusion protein, and (iii) at least one signal sequence that provides secretion of the expressed fusion protein from a cell, preferably wherein said amino acid sequence comprises at least 6 amino acid residues. Preferably, the BChE is a human BChE, including the amino acid sequence of SEQ ID NO: 2 and/or wherein the polynucleotide comprises the nucleotide sequence of SEQ ID NO: 1.

In preferred embodiments, the signal sequence directs secretion into milk or into urine. In preferred embodiments thereof, the promoter is a mammary gland-specific promoter selected from the group consisting of a WAP (whey acidic protein) promoter and a casein promoter (for secretion into milk) or the promoter is a urinary endothelium-specific promoter selected from the group consisting of a uroplakin promoter or a uromodulin promoter (for secretion into urine).

The present invention also relates to a recombinant cell that comprises the isolated polynucleotide of the invention, such as where the the cell is a MAC-T (mammary epithelial) cell or a BHK (baby hamster kidney) cell. In other embodiments, the cell is selected from the group of embryonic stem cells, embryonal carcinoma cells, primordial germ cells, oocytes, or sperm.

The present invention also encompasses a non-human mammalian embryo that comprises a polynucleotide of the invention.

The present invention is also drawn to a non-human transgenic mammal that upon lactation, expresses in its milk or urine the fusion protein of the invention, preferably wherein the BChE is fused to hSA. The mammal is preferably a mouse or a goat. The genome of said non-human transgenic mammal preferably comprises a polynucleotide of the invention. In preferred embodiments of such mammalm, the promoter is a mammary gland-specific promoter selected from the group consisting of a WAP (whey acidic protein) promoter and a casein promoter (for secretion into milk) or is a urinary endothelium-specific promoter selected from the group consisting of a uroplakin promoter or a uromodulin promoter.

In other preferred embodiments of such mammal, the genome of the mammal further comprises a DNA sequence encoding a glycosyltransferase, operably linked to a mammary gland-specific promoter, and a signal sequence that provides secretion of the glycosyltransferase. Preferred is where the mammary gland-specific promoter is a casein promoter or a whey acidic protein (WAP) promoter. Also preferred is where the genome of the mammal further comprises a DNA sequence encoding a glycosyltransferase, operably linked to a urinary gland-specific promoter, and a signal sequence that provides secretion of the glycosyltransferase. Preferably, the promoter is a urinary endothelium-specific promoter selected from the group consisting of a uroplakin promoter or a uromodulin promoter. Preferably, said mammal is a mouse or a goat.

The present invention is also drawn to a method for producing a transgenic mammal that upon lactation secretes the fusion protein of claim 16 in its milk, which method comprises allowing an embryo, into which at least one genetically-engineered DNA sequence, comprising (i) a sequence encoding said fusion protein; (ii) a mammary gland-specific promoter; and (iii) a signal sequence that provides secretion of the fusion protein into the milk of the mammal, has been introduced, to grow when transferred into a recipient female mammal, resulting in the recipient female mammal giving birth to the transgenic mammal.

Preferably, such method further comprises introducing the genetically-engineered DNA sequence into a cell of the embryo, or into a cell that will form at least part of the embryo. A preferred such procedure is wherein introducing the genetically-engineered DNA sequence comprises pronuclear or cytoplasmic microinjection of the DNA sequence or wherein introducing the genetically-engineered DNA sequence comprises combining a mammalian cell stably transfected with the DNA sequence with a non-transgenic mammalian embryo. Also preferred is the method wherein introducing the genetically-engineered DNA sequence comprises the steps of (a) introducing the DNA sequence into a non-human mammalian oocyte; and (b) activating the oocyte to develop into an embryo.

In accordance with the foregoing, the present invention also relates to a method for producing a transgenic mammal that upon lactation secretes a fusion protein of the invention, in its milk or urine, which method comprises cloning or breeding of a transgenic mammal, the genome of which comprises a DNA sequence encoding said fusion protein, operably linked to a mammary gland-specific promoter or a urinary specific promoter, wherein the sequence further comprises a signal sequence that provides secretion of the fusion protein into the milk or urine of the mammal.

The present invention further relates to a method for producing a fusion protein of the invention, comprising: (a) inducing or maintaining lactation or urination of a transgenic mammal, the genome of which comprises a DNA sequence encoding said fusion protein, operably linked to a mammary gland-specific promoter or urinary specific promoter, wherein the sequence further comprises a signal sequence that provides secretion of the fusion protein into the milk or urine of the mammal; and (b) extracting milk from the lactating mammal or urine from the urinating mammal. In a preferred embodiment thereof, such method comprises the additional step of isolating the fusion protein from the extracted milk or urine. In further preferred steps, the method comprises purifying the fusion protein. The present invention also encompasses the milk or urine of said non-human mammal comprising the fusion protein of the invention, including where the milk is whole milk or is defatted milk.

The present invention further relates to a composition comprising a fusion protein of the invention, preferably where the BChE is linked to hSA, in a pharmaceutically acceptable carrier.

The present invention also encompasses a method for treating organophosphate poisoning, comprising administering to a subject in need thereof a therapeutically effective amount of a pharmaceutical composition of the invention. The present invention further relates to a method for the treatment of post-surgical, succinyl choline-induced apnea, which comprises administering to a subject in need thereof a therapeutically effective amount of a pharmaceutical composition of the invention. The present invention further relates to a method for the treatment of cocaine intoxication, which comprises administering to a subject in need thereof a therapeutically effective amount of a pharmaceutical composition of the invention.

Production of Nucleic Acid Sequences which Encode Mutant BChE Enzymes Fused to hSA

The amino acid sequence of wildtype human BChE is set forth in U.S. Pat. No. 6,001,625 to Broomfield, et al., which is hereby incorporated herein in its entirety. This patent also discloses a mutant human BChE enzyme in which the glycine residue at the 117 position has been replaced by histidine (identified as G117H). This mutant BChE has been shown to be particularly resistant to inactivation by organophosphate compounds [Lockridge, et al. Biochemistry (1997) 36:786-795]. Accordingly, this particular form of the BChE enzyme is especially useful for treatment of pesticide or war gas poisoning. Additional variants and mutants of BChE enzymes which may be produced according the methods of the present invention are disclosed in the U.S. Pat. No. 6,001,625.

Several methods are known in the art for introducing mutations within target nucleic acid sequences which may be applied to generate and identify mutant nucleic acid sequences encoding mutant BChE enzymes. Such mutant BChE enzymes may have altered catalytic properties, temperature profile, stability, circulation time, and affinity for cocaine or other substrates and/or certain organophosphate compounds; increased or decreased formation of BChE tetramers, dimers or monomers; or other desired features. The mutant nucleic acid sequences encoding such mutant BChE enzymes may be used according to the present invention.

The template nucleic acid sequences to be used in any of the described mutagenesis protocols may be obtained by amplification using the PCR reaction (U.S. Pat. Nos. 4,683,202 and 4,683,195) or other amplification or cloning methods. The described techniques can be used to generate a wide variatey of nucleic acid sequence alterations including point mutations, deletions, insertions, inversions, and recombination of sequences not linked in nature. Note that in all cases sequential cycles of mutation and selection may be performed to further alter a mutant BChE enzyme encoded by a mutant nucleic acid sequence.

Mutations can be introduced within a target nucleic acid sequence by many different standard techniques known in the art. Site-directed in vitro mutagenesis techniques include linker-insertion, nested deletion, linker-scanning, and oligonucleotide-mediated mutagenesis (as described, for example, in “Molecular Cloning: A Laboratory Manual.” 2nd Edition” Sambrook, et al. Cold Spring Harbor Laboratory:1989 and “Current Protocols in Molecular Biology” Ausubel, et al., eds. John Wiley & Sons:1989). Error-prone polymerase chain reaction (PCR) can be used to generate libraries of mutated nucleic acid sequences (“Current Protocols in Molecular Biology” Ausubel, et al., eds. John Wiley & Sons: 1989 and Cadwell, et al. PCR Methods and Applications 1992 2:28-33). Altered BChE-encoding nucleic acid sequences can also be produced according to the methods of U.S. Pat. No. 5,248,604 to Fischer. Cassette mutagenesis, in which the specific region to be altered is replaced with a synthetically mutagenized oligonucleotide, may also be used [Arkin, et al. Proc. Natl. Acad. Sci. USA (1992) 89:7811-7815; Oliphant, et al. Gene (1986) 44:177-183; Hermes, et al. Proc. Natl. Acad. Sci. USA (1990) 87:696-700]. Alternatively, mutator strains of host cells can be employed to increase the mutation frequency of an introduced BChE encoding nucleic acid sequence (Greener, et al. Strategies in Mol. Biol. (1995) 7:32).

Another preferred method for generating and identifying mutant nucleic acid sequences encoding mutant BChE enzymes relies upon sequence or DNA “shuffling” to generate libraries of recombinant nucleic acid sequences encoding mutant BChE enzymes. The resultant libraries are expressed in a suitable host cell lines and screened for production of BChE enzymes with desired characteristics. For example, if a DNA fragment which encodes for a protein with increased binding efficiency to a ligand is desired, the BChE enzymes encoded by each of the sequence fragments of library may be tested for their ability to bind to the ligand by methods known in the art (i.e. panning, affinity).

According to the “shuffling” technique, libraries of recombinant BChE-encoding nucleic acid sequences are generated from a population of related-nucleic acid sequences that comprise sequence regions having substantial sequence identity, and which can therefore be homologously recombined in vitro or in vivo. At least two species of BChE encoding nucleic acid sequences (for example, two nucleic acid sequence variants of human BChE) are combined in a recombination system suitable for generating a sequence-recombined library, where each nucleic acid sequence insert of the library comprises a combination of a portion of the first species of BChE-encoding nucleic acid sequence with at least one adjacent portion of another species of BChE-encoding nucleic acid sequence.

The DNA shuffling process for recombination and mutation is based upon random fragmentation of a pool of related nucleic acid sequences, followed by recombination of the fragments by primeness PCR in vitro or homologous recombination in vivo. The recombined products preferably contain a portion of each of the related nucleic acid sequences. The variant nucleic acid sequence species used are fragmented by nuclease digestion, partial extension PCR amplification, PCR stuttering, or other suitable fragmenting means. The resultant fragment may be recombined by PCR in vitro. Alternatively, the variant nucleic acid sequence species may be recombined in vivo. Preferably, combinations of in vitro and in vivo shuffling are performed. In one embodiment, the first plurality of selected library members is generated by a) in vitro fragmentation of variant nucleic acids sequence species, b) introduction of the resultant fragments into a host cell or organism, and c) in vivo homologous recombination of the fragments to form “shuffled” library members.

According to the invention, the variant nucleic acid sequences which may be “shuffled” to create and identify advantageous novel BChE-encoding nucleic acid sequences include, but are not limited to, nucleic acid sequences which encode taxonomically-related, structurally-related, and/or functionally-related enzymes and/or mutated variants thereof. The taxonomically-related sequences may comprise naturally occuring homologous nucleic acid sequences representing homologous genes from different species, homologous genes from the same species, or allelic variants of the same gene within a species. In this aspect, at least two naturally-occurring genes and/or allelic variants which comprise regions of at least 50 consecutive nucleotides which have at least 70 percent sequence identity, preferably at least 90 percent sequence identity, are selected from a pool of gene sequences, such as by hybrid selection or via computerized sequence analysis using sequence data from a database. The selected sequences are obtained as isolated nucleic acid sequences, either by cloning or via DNA synthesis, and shuffled by any of the various embodiments of the invention.

Naturally—Occuring Variants of BChE

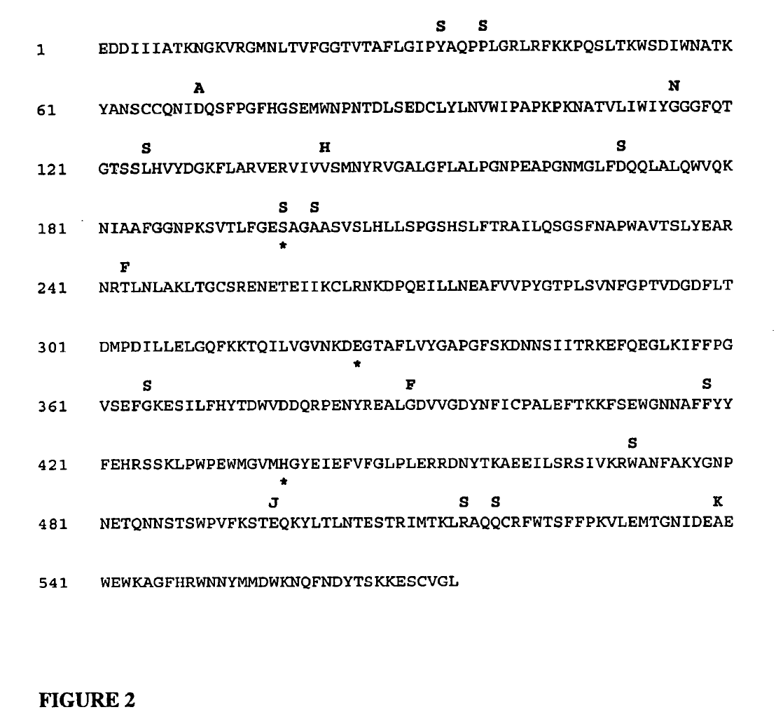

The BChE gene has four predominant allelic forms in humans, although 25 other forms responsible for various BChE genetic deficiencies are known (See Table 1 below, reproduced from the website of the American Society of Anesthesiologists, and FIG. 2). The four predominant allelic forms are designated Eu, Ea, Ef, and Es. Eu is the wildtype, fully functional allele and carries the phenotype designation EuEu or UU. The Ea allele is referred to as atypical BChE. Phenotypically, the sera of persons homozygous for this gene (EaEa=AA) are only weakly active towards most substrates for ChE and show increased resistance to inhibition of enzyme activity by dibucaine. The Ef allele also gives rise to a weakly active enzyme, but exhibits increased resistance to fluoride inhibition. The Es gene (s for silent) is associated with absence of enzyme.

The mutations in the Ea and Ef gene products cause structural alterations in the active, site of the BChE enzyme resulting in less effective catalysis compared to the native (Eu) allele. Experimentally, these mutations result in the reduction in the binding affinity (increased Km) of competitive substrates. Clinically, the phenotypes that are most susceptible to prolonged succinylcholine-induced apnea are M, SS, FF, FS, AS, AF, and UA.

Certain individuals carry an atypical BChE gene which functions normally to hydrolyze acetylcholine, but is unable to hydrolyze succinylcholine, a commonly used anesthetic. The most common variant with this problem is the atypical variant Es, for which 3-6% of the Caucasian population is heterozygous and about 0.05% is homozygous. Another variant, E1, causes the complete absence of catalytically active serum BChE in homozygotes. This type of “silent” enzyme cannot hydrolyze any ChE substrate, nor can it bind organophosphate compounds. Individuals carrying atypical or silent BChE genes are subject to prolonged apnea following surgery in which succinyl choline is administered. High frequency of atypical and silent BChE genes has been reported among Iraqui and Iranian Jews (11.3% for heterozygotes and 0.08% for homozygotes). This could explain the high frequency of reports of prolonged apnea following surgery in Israel and apparently in many other other countries. Accordingly, a recombinant BChE may administered to patients harboring these, or similar mutations, to alleviate or prevent prolonged post-surgical apnea.

FIG. 2 depicts the amino acid sequence of the mature wild-type human BChE enzyme and locations of altered residues in some BChE variants.

Assembly of Expression Constructs

The recombinant DNA methods employed in practicing the present invention are standard procedures, well-known to those skilled in the art (as described, for example, in “Molecular Cloning: A Laboratory Manual.” 2nd Edition. Sambrook, et al. Cold Spring Harbor Laboratory: 1989, “A Practical Guide to Molecular Cloning” Perbal: 1984, and “Current Protocols in Molecular Biology” Ausubel, et al., eds. John Wiley & Sons: 1989). These standard molecular biology techniques can be used to prepare the expression constructs of the invention.

TABLE 1
Structural Basis of Phenotype of Human BChE Variants

| Variant | Effect of Mutation | Phenotype Alteration |
|---|---|---|
| Atypical | D70G^{‡} | Resistance to dibucaine |
| | | inhibition |
| Fluoride-resistant | T243M | Resistance to fluoride |
| | | inhibition |
| Fluoride-resistant | G390V | Resistance to fluoride |
| | | inhibition |
| K-variant | A539T | Activity reduced by 30% |
| J-variant | E497V | Activity reduced by 70% |
| H-variant | V142M | Activity reduced by 90% |
| Sc-variant | A184V | decreased affinity for |
| | | Succinylcholine |
| Silent-1 | Frameshift at codon 117 | No activity |
| Silent-2 | Frameshift at codon 6 | No activity |
| Silent-3 | Stop codon at codon 500 | No activity |
| Silent-4 | P37S | No activity |
| Silent-5 | G365R | Trace activity |
| Silent-6 | Frameshift at codon 315 | No activity |
| Silent-8 | W471R | Trace activity |
| Silent-9 | D170E | No activity |
| Silent-10 | Q518L | Trace activity |
| Silent-11 | S198G | No activity |
| Silent-12 | Insertion of Alu element | No activity |
| | at codon 355 | |
| Silent-13 | Altered splicing of intron 2 | No activity |
| Silent-14 | L125F | Trace activity |
| Silent-16 | A201T | No activity |
| Silent-17 | Y33C | No activity |
| Silent-18 | Stop codon at codon 271 | No activity |
| Silent-19 | F418S | Trace activity |
| Silent-20 | R515C | Trace activity |
| Silent-21 | Stop codon at codon 465 | No activity |
| Unstable | G115D | Low, unstable activity |

^{‡}Numbers represent residue position in the mature wild-type human BChE enzyme, once the signal peptide has been cleaved.

Expression constructs comprise elements necessary for proper transcription and translation of a target nucleic acid sequence within the chosen host cells, including a promoter, a signal sequence to provide secretion of the translated product, and a polyadenylation signal. Such expression constructs may also contain intronic sequences or untranslated cDNA sequences intended to improve transcription efficiency, translation efficiency, and/or mRNA stability. The nucleic acid sequence intended for expression may possess its endogenous 3′ untranslated sequence and/or polyadenylation signal or contain an exogenous 3′ untranslated sequence and/or polyadenylation signal. For example the promoter, signal sequence, and 3′ intranslated sequence and polyandenylation signal of casein may be used to mediate expression of a nucleic acid sequence encoding BChE-hSA fusion protein within mammary host cells. Codon selection, where the target nucleic acid sequence of the construct is engineered or chosen so as to contain codons preferentially used within the desired host cell, may be used to minimize premature translation termination and thereby maximize expression.

The inserted nucleic acid sequence may also encode an epitope tag for easy identification and purification of the encoded polypeptide. Preferred epitope tags include myc, His, and FLAG epitope tags. The encoded epitope tag may include recognition sites for site-specific proteolysis or chemical agent cleavage to faciliate removal of the epitope tag following protein purification. For example a thrombin cleavage site could be incorporated between the recombinant BChE-hSA fusion protein and its epitope tag. Epitope tags may fused to the N-terminal end or the C-terminal end of a recombinant BChE-hSA fusion protein. Preferrably, the epitope tag is fused to the C-terminal end of a recombinant BChE: such C-terminal fusion proteins are expected to maintain cataytic activity and to retain the ability to oligomerize.

The expression constructs of the invention which provide expression of a BChE-hSA fusion protein in the desired host cells may include one or more of the following basic components:

A. Promoter

These sequences may be endogenous or heterologous to the host cell to be modified, and may provide ubiquitous (i.e., expression occurs in the absence of an apparent external stimulus and is not cell-type specific) or tissue-specific (also known as cell-type specific) expression.

Promoter sequences for ubiquitous expression may include synthetic and natural viral sequences [e.g., human cytomegalovirus immediate early promoter (CMV); simian virus 40 early promoter (SV40); Rous sarcoma virus (RSV); or adenovirus major late promoter] which confer a strong level of transcription of the nucleic acid molecule to which they are operably linked. The promoter can also be modified by the deletion and/or addition of sequences, such as enhancers (e.g., a CMV, SV40, or RSV enhancer), or tandem repeats of such sequences. The addition of strong enhancer elements may increase transcription by 10-100 fold.

For specific expression in the mammary tissue of transgenic animals, the promoter sequences may be derived from a mammalian mammary-specific gene. Examples of suitable mammary-specific promoters include: the whey acidic protein (WAP) promoter [U.S. Pat. Nos. 5,831,141 and 6,268,545, Andres, et al. Proc Natl Acad Sci USA (1987) 84(5):1299-1303], α-S1-casein [U.S. Pat. Nos. 5,750,172 and 6,013,857, PCT publication Nos. WO91/08216 and WO93/25567], αS2-casein, β-casein [U.S. Pat. No. 5,304,489; Lee, et al. Nucleic Acids Res. (1988) 16:1027-1041], kappa.-casein [Baranyi, et al. Gene (1996) 174(1):27-34; Gutierrez, et al. Transgenic Research (1996) 5(4):271-279], β-lactoglobin [McClenaghan, et al. Biochem J (1995) 310(Pt2):637-641], and α-lactalbumin [Vilotte, et al. Eur. J. Biochem. (1989) 186: 43-48; PCT publication No. WO88/01648].

For specific expression in the urinary endothelium of transgenic animals, the promoter sequences may be derived from a mammalian urinary endothelium-specific gene. Examples of suitable urinary endothelium-specific promoters include the uroplakin II promoter [Kerr, et al. Nature Biotechnology (1998) 16(1):75-79], and the uromodulin promoter [Zbikowska, et al. Biochem J (2002) 365(Ptl):7-1 1; Zbikowska, et al. Transgenic Res 2002 11(4):425-435].

B. Intron Inclusion

Nucleic acid sequences containing an intronic sequences (e.g., genomic sequences) may be expressed at higher levels than intron-less sequences. Hence, inclusion of intronic sequences between the transcription initiation site and the translational start codon, 3′ to the translational stop codon, or inside the coding region of the BChE-hSA fusion protein-encoding nucleic acid sequence may result in a higher level of expression.

Such intronic sequences include a 5′ splice site (donor site) and a 3′ splice site (acceptor site), separated by at least 100 base pairs of non-coding sequence. These intronic sequences may be derived from the genomic sequence of the gene whose promoter is being used to drive BChE expression, from a native BChE gene, or another suitable gene. Such intronic sequences should be chosen so as to minimize the presence of repetitive sequences within the expression construct, as such repetitive sequences may encourage recombination and thereby promote instability of the construct. Preferrably, these introns can be positioned within the BChE-encoding nucleic acid sequence so as to approximate the intron/exon structure of the native human BChE gene.

C. Signal Sequences

Each expression construct will additionally comprise a signal sequence to provide secretion of the translated recombinant BChE from the host cells of interest (e.g., mammary or uroepithelial cells, or mammalian cell culture). Such signal sequences are naturally present in genes whose protein products are normally secreted secreted. The signal sequences to be employed in the invention may be derived from a BChE gene, from a gene specifically expressed in the host cell of interest (e.g., casein or uroplakin gene), or from another gene whose protein product is known to be secreted (e.g., from hSA, alkaline phosphatase, mellitin, the immunoglobulin light chain protein Ig.kappa., and CD33); or may be synthetically derived.

D. Termination Region

Each expression construct will additionally comprise a nucleic acid sequence which contains a transcription termination and polyandenylation sequence. Such sequences will be linked to the 3′ end of the BChE-hSA fusion protein-encoding nucleic acid sequence. These sequences may comprise the 3′-end and polyadenylation signal from the gene whose 5′-promoter region is driving BChE-hSA fusion protein expression (e.g., the 3′ end of the goat β-casein gene). Alternatively, such sequences will be derived from genes in which the sequences have been shown to regulate post-transcriptional mRNA stability (e.g., those derived from the bovine growth hormone gene, the β-globin genes, or the SV40 early region).

E) Other Features of the Expression Constructs

The BChE-hSA fusion protein-encoding nucleic acid sequences of interest may be modified in their 5′ or 3′ untranslated regions (UTRs), and/or in regions coding for the N-terminus of the BChE enzyme so as to preferentially improve expression. Sequences within the BChE-hSA fusion protein-encoding nucleic acid sequence may be deleted or mutated so as to increase secretion and/or avoid retention of the BChE enzyme product within the cell, as regulated, for example, by the presence of endoplasmic reticulum retention signals or other sorting inhibitory signals.

In addition, the expression constructs may contain appropriate sequences located 5′ and/or 3′ of the BChE-hSA fusion protein-encoding nucleic acid sequences that will provide enhanced integration rates in transduced host cells [e.g., ITR sequences as per Lebkowski, et al. Mol. Cell. Biol. (1988) 8:3988-3996]. Furthermore, the expression construct may contain nucleic acid sequences that possess chromatin opening or insulator activity and thereby confer reproducible activation of tissue-specific expression of a linked transgene. Such sequences include Matrix Attachment Regions (MARs) [McKnight, et al. Mol Reprod Dev (1996) 44(2):179-184 and McKnight, et al. Proc Natl Acad Sci USA (1992) 89:6943-6947]. See also Ellis, et al., PCT publication No.: WO95/33841 and Chung and Felsenfield, PCT publication No.: WO96/04390.

The expression contructs further comprise vector sequences which facilitate the cloning and propagation of the expression constructs. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest. Prolonged expression of the encoded BChE-hSA fusion protein in in vitro cell culture may be achieved by the use of vectors sequences that allow for autonomous replication of an extrachromosomal construct in mammalian host cells (e.g., EBNA-1 and oriP from the Epstein-Barr virus).

The expression constructs used for the generation of transgenic animals may be linearized by restriction endonuclease digestion prior to introduction into a host cell. In a variant of this method, the vector sequences are removed prior to introduction into host cells, such that the introduced linearized fragment is comprised solely of the BChE-hSA fusion protein-encoding sequence, 5′-end regulatory sequences (e.g., the promoter), and 3′-end regulatory sequences (e.g., the 3′ transcription termination and polyandenylation sequences), and any flanking insulators or MARs. A cell transformed with such a fragment will not contain, for example, an E. coli origin or replication or a nucleic acid molecule encoding an antibiotic-resistance protein (e.g., an ampicillin-resistance protein) used for selection of transformed prokaryotic cells.

In another variant of this method, the restriction digested expression construct fragment used to transfect a host cell will include a BChE-hSA fusion protein-encoding sequence, 5′ and 3′ regulatory sequences, and any flanking insulators or MARs, linked to a nucleic acid sequence encoding a protein capable of conferring resistance to a antibiotic useful for selection of transfected eukaryotic cells (e.g., neomycin or puromycin).

Generation of Transfected Cell Lines In Vitro

The expression constructs of the invention may be transfected into host cells in vitro. Preferred in vitro host cells are mammalian cell lines including BHK, MDCK, Hu609, MAC-T (U.S. Pat. No. 5,227,301), R1 embryonic stem cells, embryonal carcinoma cells, COS, or HeLa cells. Protocols for in vitro culture of mammalian cells are well established in the art [see for example, Animal Cell Culture: A Practical Approach 3rd Edition. J. Masters, ed. Oxford University Press and Basic Cell Culture 2nd Edition. Davis, J. M. ed. Oxford University Press (2002)]. Techniques for transfection are well established in the art and may include electroporation, microinjection, liposome-mediated transfection, calcium phosphate-mediated transfection, or virus-mediated transfection [see for example, Artificial self-assembling systems for gene delivery. Feigner, et al., eds. Oxford University Press (1996); Lebkowski, et al. Mol Cell Biol 1988 8(10):3988-3996; “Molecular Cloning: A Laboratory Manual.” 2nd edition. Sambrook, et al. Cold Spring Harbor Laboratory: 1989; and “Current Protocols in Molecular Biology” Ausubel, et al., eds. John Wiley & Sons: 1989). Where stable transfection of the host cell lines is desired, the introduced DNA preferably comprises linear expression construct DNA, free of vector sequences, as prepared from the expression constructs of the invention. Transfected in vitro cell lines may be screened for integration and copy number of the expression construct. For such screening, the genomic DNA of a cell line is prepared and analyzed by PCR and/or Southern blot.

Transiently and stably transfected cell lines may be used to evaluate the expression contructs of the invention as detailed below, and to isolate recombinant BChE-hSA fusion protein and/or glysosyltransferase proteins. Where the expression construct comprises a ubiquitous promoter any of a number of established mammalian cell culture lines may be transfected. Where the expression construct comprises a tissue-specific promoter, the host cell line should be compatible with the tissue specific promoter (e.g., uromodulin promoter containing expression constructs may be transfected into baby hamster kidney BHK cells).

Stably transfected cell lines may be also used to generate transgenic animals. For this use, the recombinant proteins need not be expressed in the in vitro cell line.

Evaluation of Expression Constructs

Prior to the generation of transgenic animals using the expression constructs of the invention, expression construct functionality can be determined using transfected in vitro cell culture systems. Genetic stability of the expression constructs, degree of secretion of the recombinant protein(s), and physical and functional attributes of the recombinant protein(s) can be evaluated prior to the generation of transgenic animals.

Where the expression construct comprises a ubiquitous promoter any of a number of established mammalian cell culture lines may be transfected. Where the expression construct(s) comprises mammary gland or urinary endothelium-specific promoters, mammary epithelium and bladder cell lines can be transfected. For example, the hamster kidney cell line BHK-21 (C-13) (ATCC #CCl-10) [Sikri, et al. Biochem. J. (1985) 225:481-486] and the dog kidney cell line MDCK (ATCC #CCL-34) can be used to test the functionality of uromodulin promoter containing expression constructs. The human urothelium cell line Hu609 [Stacey, et al. Mol. Carcinog. (1990) 3:216-225] may used to test the functionality of uroplakin promoter containing expression constructs.

To determine if cell lines transfected with the BChE-hSA fusion protein-encoding expression constructs of the invention are producing recombinant BChE-hSA fusion protein, the media from transfected cell cultures can be tested directly for the presence of the secreted protein by Western blotting analysis using anti-BChE antibody (Monsanto, St. Louis, Mo.) or assessed using an activity assay [Ellman, et al. Biochem. Pharmacol. (1.961) 7:88-95]. Where a cell lines is stably transfected and has been shown to produce catalyticaly active recombinant protein, the cell lines may be used for large scale culture and purification of the recombinant protein. Such cell lines may also be used in the generation of transgenic animals.

Generation of Transgenic Mammals

Protocols for the generation of non-human transgenic mammals are well established in the art [see, for example, Transgenesis Techniques Murphy, et al., Eds., Human Press, Totowa, N.J. (1993); Genetic Engineering of Animals A. Puhler, Ed. VCH Verlagsgesellschaft, Weinheim, N.Y. (1993); and Transgenic Animals in Agriculture Murray, et al., eds. Oxford University Press]. For example, efficient protocols are available for the production of transgenic mice [Manipulating the Mouse Embryo 2nd Edition Hogan, et al. Cold Spring Harbor Press (1994) and Mouse Genetics and Transgenics: A Practical Approach. Jackson and Abbott, eds. Oxford University Press (2000)], transgenic cows (U.S. Pat. No. 5,633,076), transgenic pigs (U.S. Pat. No. 6,271,436), and transgenic goats (U.S. Pat. No. 5,907,080). Preferred examples of such protocols are summarized below. It will be appreciated that these examples are not intended to be limiting, and that transgenic non-human mammals comprising the expression constructs of the invention, as created by these or other protocols, necessarily fall within the scope of the invention.

Transgenic animals may be generated using stably transfected host cells derived from in vitro transfection. Where said host cells are pluripotent or totipotent, such cells may be used in morula aggregation or blastocyst injection protocols to generate chimeric animals. Preferred pluripotent/totipotent stably transfected host cells include primoridal germ cells, embryonic stem cells, and embryonal carcinoma cells. In a morula aggregation protocol, stably transfected host cells are aggregated with non-transgenic morula-stage embryos. In a blastocyst injection protocol, stably transfected host cells are introduced into the blastocoelic cavity of a non-transgenic blastocyst-stage embryo. The aggregated or injected embryos are then transferred to a pseudopregnant recipient female for gestation and birth of chimeras. Chimeric animals in which the transgenic host cells have contibuted to the germ line may be used in breeding schemes to generate non-chimeric offspring which are wholly transgenic.

In an alternative protocol, such stably transfected host cells may be used as nucleus donors for nuclear transfer into recipient oocytes (as per Wilmut, et al. Nature (1997) 385: 810-813). For nuclear transfer, the stably transfected host cells need not be pluripotent or totipotent. Thus, for example, stably transfected fetal fibroblasts can be used [e.g., Cibelli, et al. Science (1998) 280: 1256-8 and Keefer, et al. Biology of Reproduction (2001) 64:849-856]. The recipient oocytes are preferrably enucleated prior to transfer. Following nuclear transfer, the oocyte is transferred to a pseudopregnant recipient female for gestation and birth. Such offspring will be wholly transgenic (that is, not chimeric).

In another alternative protocol, transgenic animals are generated by direct introduction of expression construct DNA into a recipient oocyte, zygote, or embryo. Such direct introduction may be achieved by pronuclear microinjection [Wang, et al. Molecular Reproduction and Development (2002) 63:437-443], cytoplasmic microinjection [Page, et al. Transgenic Res (1995) 4(6):353-360], retroviral infection [e.g., Lebkowski, et al. Mol Cell Biol (1988) 8(10):3988-3996], or electroporation (“Molecular Cloning: A Laboratory Manual. Second Edition” by Sambrook, et al. Cold Spring Harbor Laboratory: 1989).

For microinjection and electroporation protocols, the introduced DNA should comprise linear expression construct DNA, free of vector sequences, as prepared from the expression constructs of the invention. Following DNA introduction and any necessary in vitro culture, the oocyte, zygote, or embryo is transferred to a pseudopregnant recipient female for gestation and birth. Such offspring may or may not be chimeric, depending on the timing and efficiency of transgene integration. For example, if a single cell of a two-cell stage embryo is microinjected, the resultant animal will most likely be chimeric.

Transgenic animals comprising two or more independent transgenes can be made by introducing two or more different expression constructs into host cells using any of the above described methods.

The presence of the transgene in the genomic DNA of an animal, tissue, or cell of interest, as well as transgene copy number, may be confirmed by techniques well known in the art, including hybridization and PCR techniques.

Some of the transgensis protocols result in the production of chimeric animals. Chimeric animals in which the transgenic host cells have contributed to the tissue-type wherein the promoter of the expression construct is active (e.g., mammary gland for WAP promoter) may be used to characterize or isolate recombinant BChE-hSA fusion protein and/or glucosyltransferase enzymes. More preferably, where the transgenic host cells have contibuted to the germ line, chimeras may be used in breeding schemes to generate non-chimeric offspring which are wholly transgenic.

Wholly transgenic offspring, whether generated directly by a transgensis protocol or by breeding of a chimeric animals, may be used for breeding purposes to maintain the transgenic line and to characterize or isolate recombinant BChE-hSA fusion protein and/or glucosyltransferase enzymes. Where transgene expression is driven by a urinary endothelium-specific promoter, urine of transgenic animals may be collected for purification and characterization of recombinant enzymes. Where transgene expression is driven by a mammary gland-specific promoter, lactation of the transgenic animals may be induced or maintained, where the resultant milk may be collected for purification and characterization of recombinant enzymes. For female transgenics, lactation may be induced by pregnancy or by administration of hormones. For male transgenics, lactation may be induced by administration of hormones (see for example Ebert, et al. Biotechnology (1994) 12:699-702). Lactation is maintained by continued collection of milk from a lactating transgenic.

Purification of Recombinant BChE-hSA Fusion Protein

Recombinant BChE-hSA fusion protein may be isolated from the culture medium of BChE-hSA fusion protein-secreting transfected cells in vitro, from the milk of transgenic animals expressing BChE-hSA fusion protein in mammary gland, or from the urine of transgenic animals expressing BChE-hSA fusion protein in urinary endothelium using a procainamide affinity chromatography protocol (as described in Lockridge, et al. Biochemistry (1997) 36:786-795). For purification from culture medium, the medium is centrifuged or filtered to remove cellular debris prior to application to the procainamide column. The medium may also be concentrated by ultrafiltration. For purification from milk, tangential flow filtration clarification may be used to remove caseins and fat prior to application to the procainamide column. For purification from urine, the urine is first centrifuged to remove cell debris. Then the urine is diluted to reduce salt concentration, as measured by conductivity. The resulting solution is then applied to the column.

To provide enhanced purity of recombinant BChE-hSA fusion protein, additional steps such as blue Sephasose CL-6B chromatography or ion exchange chromatography in combination with ammonium sulfate fractionation may be performed. Enzyme purity may be evaluated by reverse phase HPLC. Purified recombinant BChE-hSA fusion protein may be further separated on Sephacryl S-300.

Assays to Characterize BChE-hSA Fusion Protein

The assays described here may be used to characterize variant BChE-hSA fusion proteins as produced by the described mutagenesis protocols prior to expression construct assembly, and/or to characterize recombinant BChE-hSA fusion protein collected from culture medium of transfected cells or from the milk or urine of transgenic animals. These assays allow for characterization of BChE-hSA fusion protein activity, stability, structural characteristics, and in vivo function.

Various methods for in vitro BChE enzymatic activity assays are described in the art (for example, Lockridge and La Du, J Biol Chem (1978) 253:361-366; Lockridge, et al. Biochemistry (1997) 36:786-795; Plattborze and Broomfield, Biotechnol. Appl. Biochem. (2000) 31:226-229; and Blong, et al. Biochem J (1997) 327:747-757). Samples can be tested for the presence of enzymatically active recombinant BChE by using the activity assay of Ellman (Ellman, et al. Biochem Pharmacol (1961) 7:88). Levels of BChE activity can be estimated by staining non-denaturing 4-30% polyacrylamide gradient gels with 2 mM echothiophate iodide as substrate (as described in Lockridge, et al. Biochemistry (1997) 36:786-795), where this method is a modification of the same assays using 2 mM butrylythiocholine as substrate (from Karnovsky and Roots, J Histochem Cytochem (1964) 12:219). Using these methods, the catalytic properties of a BChE-hSA fusion protein, including Km, Vmax, and kcat values, may be determined using butyrylthiocholine or acetylthiocholine as substrate. Other methodologies known in the art can also be used to assess ChE function, including electrometry, spectrophotometry, chromatography, and radiometric methodologies.

Purified recombinant BChE-hSA fusion protein may be separated on Sephacryl S-300. Relative amounts of BChE-hSA fusion protein can also be estimated by staining non-denaturing 4-30% polyacrylamide gradient gels with 2 mM echothiophate iodide as substrate (as described in Lockridge, et al. Biochemistry (1997) 36:786-795). A panel of monoclonal antibodies may be used to characterize the functional domains of the recombinant BChE-hSA fusion protein.

A competitive enzyme-linked immunosorbent assay (ELISA) may be used to quantitate the concentration of BChE-hSA fusion protein in a sample. This assay is based in a poly-clonal rabbit anti-human BChE antibody coupled to biotin, where binding of the biotinylated antibody to immobilized BChE antigen is competitively inhibited by an added standard or the test sample. The amount of label-bound antibody is inversely related to the concentration of BChE in the test sample.

The recombinant BChE-hSA fusion protein may be further characterized by standard techniques well known in the art, including N-terminal sequencing, determination of carbohydrate content (especially terminal sialic acid content), tryptic and carbohydrate mapping, and determination of in vitro stability. For example, the composition, distribution, and structure of monosaccharide and oligosaccharide moieties of the recombinant BChE-hSA fusion protein may be analyzed as described in Saxena, et al. Biochemistry (1997) 36:7481-7489.

Potential clinical effectiveness of a recombinant BChE-hSA fusion protein sample against organophosphate poisoning or cocaine toxicity can be assessed both in vitro and in vivo. For example, in vitro OPAH activities of the potential substrates soman, sarin and tabun can be measured in a pH stat using a solution of the test recombinant BChE-hSA fusion protein. The activity of recombinant BChE-hSA fusion protein against VX and echothiophate can be measured in a microtitre plate using a variation of the Ellman method, with the OP compound replacing the butyrylthioline as substrate. Enzyme-catalyzed hydrolysis of cocaine can be recorded on a temperature-equilibrated Gilford Spectrophotometer at 240 nm (Xie, et al. Mol. Pharmacol. 1999 55:83-91).

The in vivo half life and protective effect versus organophosphate poisoning of a recombinant BChE-hSA fusion protein sample may be assessed in animal models, such as rodents or primates (for example as in Raveh, et al. Toxicol. Applied Pharm. (1997) 145:43-53; Broomfield, et al. J Pharmacol Exp Ther (1991) 259:633-638; Brandeis, et al. Pharmacol Biochem Behav (1993) 46:889-896; Ashani, et al Biochem Pharmacol (1991) 41:37-41; and Rosenberg, et al. Life Sciences (2002) 72:125-134). Peak blood BChE-level may be determined following intramuscular injection of recombinant BChE-hSA fusion protein as described in Raveh, et al. Biochem Pharmacol (1993) 45(12):2465. Similarly, the in vivo half life and protective effect versus cocaine toxicity of a recombinant BChE-hSA fusion protein sample may be assessed in animal models (for example, as in Hoffman, et al. J Toxicol Clin Toxicol (1996) 34:259-266 and Lynch et al Toxicol Appl Pharmacol (1997) 145:363-371).

Once the in vivo stability and efficacy of a recombinant BChE-hSA fusion protein preparation has been verified in animal models, such preparations may be used for the treatment of various conditions, including organophopsate poisoning, post-surgical succinyl-choline induced apnea, or cocaine intoxication.

Treatment of Organophosphate Poisoning and Other Conditions

Exposure to organophosphate compounds can result in a wide variety of symptoms depending on the toxicity of the compound, the amount of compound involved in the exposure, the route of exposure, and the duration of the exposure. In mild cases, symptoms such as tiredness, weakness, dizziness, runny nose, bronchial secretions, nausea, and blurred vision may appear. In moderate cases, symptoms may include tightness in the chest, headache, sweating, tearing, drooling, excessive perspiration, vomiting, tunnel vision, and muscle twitching. In severe cases, symptoms include abdominal cramps, involuntary urination and diarrhea, muscular tremors, convulsions, staggering gait, pinpoint pupils, hypotension (abnormally low blood pressure), slow heartbeat, breathing difficulty, coma, and possibly death. Severe cases of organophosphate poisoning are observed after continued daily absorption of organophosphate pesticides, or from exposure to the most toxic organophosphate compounds used as chemical warfare agents. When symptoms of organophosphate poisoning first appear, it is generally not possible to tell whether a poisoning will be mild or severe. In many instances, when the skin is contaminated, symptoms can quickly go from mild to severe even though the area is washed. Some of the most toxic organophosphate compounds are those used as war gases. These compounds include tabun (GA), methyl parathion, sarin (GB), VX, soman (GD), diisopropylfluorophosphate, and PB. These compounds are easily absorbed through the skin, and may be inhaled or ingested. The symptoms of nerve gas poisoning are usually similar, regardless of the route of introduction.

Some of the most commonly used organophosphate pesticides include acephate (Orthene), Aspon, azinphos-methyl (Guthion), carbofuran (Furadan, F formulaltion), carbophenothion (Trithion), chlorfenvinphos (Birlane), chlorpyrifos (Dursban, Lorsban), coumaphos (Co-Ral), crotoxyphos (Ciodrin, Ciovap), crufomate (Ruelene), demeton (Systox), diazinon (Spectracide), dichlorvos (DDVP, Vapona), dicrotophos (Bidrin), dimethoate (Cygon, De-Fend), dioxathion (Delnav), disulfoton (Di-Syston), EPN, ethion, ethoprop (Mocap), famphur, fenamiphos (Nemacur), fenitrothion (Sumithion), fensulfothion (Dasanit), fenthion (Baytex, Tiguvon), fonofos (Dyfonate), isofenfos (Oftanol, Amaze), malathion (Cythion), methamidophos (Monitor), methidathion (Supracide), methyl parathion, mevinphos (Phosdrin), monocrotophos, naled (Dibrom), oxydemeton-methyl (Meta systox-R), parathion (Niran, Phoskil), phorate (Thimet), phosalone (Zolonc), phosmet (Irnidan, Prolate), phosphamidon (Dimecron), temephos (Abate), TEPP, terbufos (Counter), tetrachlorvinphos (Rabon, Ravap), and trichlorfon (Dylox, Neguvon).

Commonly used carbamate pesticides include aldicarb (Temik), bendiocarb (Ficam), bufencarb, carbaryl (Sevin), carbofuran (Furadan), formetanate (Carzol), methiocarb (Mesurol), methomyl (Lannate, Nudrin), oxamyl (Vydate), pirimicarb (pinmicarb, Pirimor) and propoxur (Baygon).

The present invention encompasses a method for the treatment of organophosphate poisoning comprising, administering to a subject in need thereof a therapeutically effective amount of recombiant BChE-hSA fusion protein. The invention includes treatment of and amelioration of the symptoms resulting from exposure to organophosphate compounds, as well as methods of preventing symptoms of exposure to these compounds. Such methods involve administering to a subject an amount of recombinant BChE-hSA fusion protein effective to protect against these symptoms, prior to exposure of the subject to an organophosphate compound.

The invention is also directed to methods for treating post-surgical, succinyl choline-induced apnea, and cocaine intoxication. These methods comprise administration to a subject suffering from post-surgical, succinyl choline-induced apnea or cocaine intoxication an effective amount of recombinant BChE-hSA fusion protein.

### EXAMPLES

### Example 1

### Production of Recombinant BChE in Cell Culture

1.1 Assembly of Expression Constructs

Standard recombinant DNA methods employed herein have been described in detail (see, for example, in “Molecular Cloning: A Laboratory Manual.” 2nd Edition. Sambrook, et al. Cold Spring Harbor Laboratory:1989, “A Practical Guide to Molecular Cloning” Perbal: 1984, and “Current Protocols in Molecular Biology” Ausubel, et al., eds. John Wiley & Sons:1989). All DNA cloning manipulations were performed using E. coli STBII competent cells (Canadian Life Science, Burlington, Canada). Restriction and modifying enzymes were purchased from New England BioLabs (Mississauga, ON, Canada). All chemicals used were reagent grade and purchased from Sigma Chemical Co (St. Louis, Mo.), and all solutions were prepared with sterile and nuclease-free WFI water (Hyclone, Tex.). Construct integrity was verified by DNA sequencing analysis provided by McMaster University (Hamilton, ON, Canada). Primers were synthesized by Sigma Genosys (Oakville, ON, Canada). PCR was performed using Ready-To-Go PCR beads (Pharmacia Biotech, Baie d'Urf, PQ, Canada) or the High Fidelity PCR kit (Roche Diagnostics Canada, Laval, Canada).

In the expression contructs for the expression of recombinant BChE in in vitro cell culture, a sequence encoding human BChE was under the transcriptional control of a strong constitutive promoter and was linked to a signal sequence to provide secretion of the recombinant protein from the cells.

pCMV/IgKBChE

The human BChE cDNA was PCR amplified from a cDNA clone (ATCC #65726), with a sense primer Acb787 (5′ AGA GAG GGG GCC CAA GAA GAT GAC ATC ATA ATT G 3′) (SEQ ID NO: 3) containing an ApaI site (underlined) and a partial immunoglobulin kappa (Ig.kappa.) signal sequence, and an antisense primer Acb786 (5′ CTG CGA GTT TAA ACT ATT AAT TAG AGA CCC ACA C 3′) (SEQ ID NO: 4) including a PmeI site (underlined) and partial 3′ sequence of the human BChE cDNA. The PCR product was digested with ApaI and PmeI, purified using GFX matrix (Pharmacia Biotech, Baie d'Urf,PQ, Canada) and ligated into ApaI and PmeI digested pSecTag/MaSpI to generate pCMV/IgKBChE.

The construction of pSecTag/MaSp1 is described in Lazaris, et al. Science (2002) 295: 472-476. Briefly, this plasmid contains the coding sequence of the spider silk protein gene MaSp1 cloned into the vector pSecTag (Invitrogen). ApaI and PmeI digestion of pSecTag/MaSpI removes the MaSp1 sequences as well as the His epitope tag sequences of the pSecTag vector. The remaining pSecTag vector sequences comprise the CMV promoter, the mouse IgK signal sequence, and bovine growth hormone termination and polyadenylation sequence.

The final expression construct pCMV/IgKBChE contains the sequence encoding mature human BChE, linked to the mouse IgK signal sequence, under the transcriptional control of the cytomegalovirus promoter (CMV), as well as the bovine growth hormone termination and polyadenylation sequences for efficient transcription termination and transcript stability.

pCMV/BChE

pCMV/IgKBChE was digested with NheI and the ends were filled in using T4 DNA polymerase in the presence of dNTPs. This linearized vector then was digested with XbaI. This NheI (blunt-ended)-XbaI fragment was ligated to the BglII (blunt-ended)-XbaI fragment of the human BChE cDNA to generate pCMV/BChE, with BChE's own signal sequence retained.

pCMV/BChE/hSA

PCR was performed using pCMV/BChE as a template with a sense primer Acb710 (5′ GTG TAA CTC TCT TTG GAG AAA G 3′) (SEQ ID NO: 5) containing a portion of 5′ BChE sequence and an antisense primer Acb853 (5′ TAT AAG TTT AAA CAT ATA ATT GGA TCC TCC ACC TCC GCC TCC GAG ACC CAC ACA ACT TTC TTT CTT G 3′) (SEQ ID NO: 6) containing a PmeI site (underlined), a BamHI site (italic), a (Gly)6-Ser linker (bolded) followed by a portion of 3′ BChE sequence. The PCR product was digested with XbaI and PEmeI, and ligated to XbaI and PmeI digested pCMV/BChE to generate pCMV/BChEmd.

PCR was performed using Marathon-ready human liver cDNA pool (Clontech) as a template with a sense primer Acb854 (5′ ATA TAA GGA TCC GAT GCA CAC AAG AGT GAG GTT GCT CAT C₃′) (SEQ ID NO: 7) containing a BamHI site (underlined) and partial sequence from the hSA cDNA 5′ end (Genbank V00495, without the signal sequence), and an antisense prime r Acb855 (5′ ATT TAA GTT TAA ACT CAT TAT AAG CCT AAG GCA GCT TGA CTT GC 3′) (SEQ ID NO: 8) including a PmeI site (underlined) and partial sequence from the hSA cDNA 3′ end. This PCR product was digested with BamHI and PmeI and inserted into BamHI and PmeI digested pCMV/BChEmd to generate the final construct, pCMV/BChE/hSA. This expression construct encodes a BChE-hSA fusion protein.

1.2. Transfection and Selection of Stable Cell Lines.

Preparation of Expression Constructs for Transfection:

The constructs pCMV/IgKBChE and pCMV/BChE/hSA were digested with FspI, and the resultant FspI-digested linear DNA, was prepared and used for transfection. Briefly, circular expression construct DNA was purified by the cesium chloride gradient technique. This purified DNA was restricted with FspI, precipitated, and resuspended in sterile deionized water.

Stably Transfected MAC-T Cell Lines Expressing Recombinant BChE:

MAC-T cells (ATCC #CRL 10274, U.S. Pat. No. 5,227,301) were seeded at a density of 5×10⁵ cells per 100 mm dish. On the following day, cells were transfected with Lipofectamine PLUS Reagent (Invitrogen) as per the manufacturer's recommendations with 4 μg of the linearized pCMV/IgKBChE construct. Briefly, the DNA was diluted to a final volume of 750 μL with DMEM (Invitrogen) and 20 μL of PLUS Reagent was added to the mixture. The Lipofectamine was diluted to a final volume of 750 μL with DMEM. After incubation at ambient temperature for 15 min, the Lipofectamine and DNA mixtures were combined and complexes allowed to form for 15 min at room temperature.

The lipid-DNA complex mixture was applied to the cells, and the cells allowed to incubate for 3 hrs at 37° C. under 5% CO₂. The cells were then cultured for another 24 h in fresh medium containing 20% fetal bovine serum (FBS, Invitrogen). Subsequently, stably transfected cells were selected in DMEM containing 10% FBS, 5 μg/ml insulin (Sigma), and 100 μg/ml hygromycin B (Invitrogen). Colonies surviving selection were picked 7 to 14 days following transfection and expanded further.

The level of BChE activity in cell culture media from pCMV/IgKBChE transfected MAC-T cells was evaluated by measuring butyrylthiocholine iodide hydrolysis (see Ellman, et al. Biochem Pharmacol (1961) 7:88) using a commercially available test (Sigma). The assay was performed according to the manufacturer's recommendations. The resulting activity values in units/ml were converted to mg of active BChE by using the relationship: 1 mg of active BChE=720 units. From over 100 clones tested, the one demonstrating the highest BChE activity, as tested by the Ellman activity assay was further evaluated in roller bottles containing serum-free DMEM. The amount of BChE activity under these conditions was estimated at 0.56 units per million cells (U/1 06) per 24 hours.

A master cell bank was generated and used to initiate a hollow fiber bioreactor production run (Biovest, CP2500 model). Hollow fibre production of stable transfectants was established for large-scale production of recombinant BChE.

Stably Transfected MAC-T Cell Lines Expressing a Recombinant BChE-hSA Fusion:

MAC-T cells were seeded at a density of 2.5.times.10⁵ cells per 100 mm dish. On the following day, cells were transfected with Lipofectamine Reagent (Invitrogen) with 10 μg of the linearized pCMV/BChE-hSA construct. Briefly, the DNA was diluted to a final volume of 500 μL with DMEM (Invitrogen) and 60 μL of Lipofectamine was diluted to a final volume of 500 μL with DMEM. The two solutions were combined, vortexed for 10 sec and the complexes were allowed to form at room temperature for 30 min. DMEM was added to the lipid-DNA mixture up to a final volume of 5 ml. The mixture was then applied to the cells and allowed to incubate overnight at 37° C. under 5% CO₂. The cells were then cultured for another 24 h in DMEM containing 10% FBS, 5 μg/ml insulin (Sigma).

Stably transfected cells were selected in DMEM containing 10% FBS, 5 μg/ml insulin (Sigma), and 100 μg/ml hygromycin B (Invitrogen). Colonies surviving selection were picked 7 to 14 days following transfection and expanded further.

The level of BChE activity in cell culture media from pCMV/BChE-hSA transfected MAC-T cells was evaluated using a commercially available test (Sigma). From over 100 clones tested, the one demonstrating the highest BChE activity was further evaluated in roller bottles containing serum-free DMEM. The amount of BChE activity under these conditions was estimated at 0.17 units per million cells (U/10⁶) per 24 hours. Thus, it was successfully demonstrated that the recombinant BChE-hSA fusion protein is active.

Stably Transfected BHK Cell Lines Expressing a Recombinant BChE-hSA Fusion:

These lines were generated using the same procedure for stable transfection of MAC-T cells with pCMV/BChE-hSA, with the exception that the cells were BHK (Baby Hamster Kidney) cells (supplied by Dr. G. Matleshewski of McGill University, also available from the ATCC, clone #CCl-10) and the selection media contained DMEM with 10% FBS and 300 μg/ml hygromycin B (Invitrogen). Colonies surviving selection were picked 7 to 14 days following transfection and expanded further.

The level of BChE activity in cell culture media from pCMV/BChE-hSA transfected BHK cells was evaluated using a commercially available test (Sigma). From over 100 clones tested, the one demonstrating the highest BChE activity was further evaluated in roller bottles containing serum-free DMEM. The amount of BChE activity under these conditions was estimated at 0.73 units per million cells (U/10⁶) per 24 hours.

1.3. Detection of Recombinant BChE in Culture Media of Transfected Cells.

Western blotting analysis of non-denaturing PAGE gels and denaturing SDS-PAGE gels was used to detect the presence of recombinant BChE in cell culture media. Cell culture media from pCMV/IgKBChE transfected MAC-T cells, and pCMV/BChE/hSA transfected MAC-T or BHK cells, was electrophoresed on non-denaturing and denaturing pre-cast 4-20% TRIS-glycine gels (Invitrogen). The samples were then transferred by electroblotting onto nitrocellulose membranes (Bio-Rad). Recombinant BChE on the membranes was detected using rabbit polyclonal antibodies raised against BChE (DAKO) at a dilution of 1:1000 and goat anti-rabbit horseradish peroxidase conjugated second antibody. Detection was performed according to manufacturer's protocol for enhanced chemiluminescence (ECL) detection (Amersham Pharmacia).

In such analyses, the anti-BChE antibodies specifically detected a protein of the appropriate molecular weight in cell culture media from transfected cells. These results confirmed the production of recombinant BChE, and of the recombinant BChE-hSA fusion protein, in transfected cell lines in in vitro culture.

1.4 BChE Activity Gels

20 μL of samples of cell culture media from pCMV/IgKBChE transfected MAC-T cells, and pCMV/BChE/hSA transfected MAC-T and BHK cells, was electrophoresed on native 4-20% pre-cast TRIS-glycine gels at 100-125 V overnight and at 4° C. The gels were then stained for BChE activity with 2 mM of butyrylthiocholine iodide according to the Karnovsky and Roots method (Karnovsky and Roots, Histochem. Cytochem. (1964) 12:219-221). The staining procedure was performed at ambient temperature for two to six hours until the active protein bands were revealed.

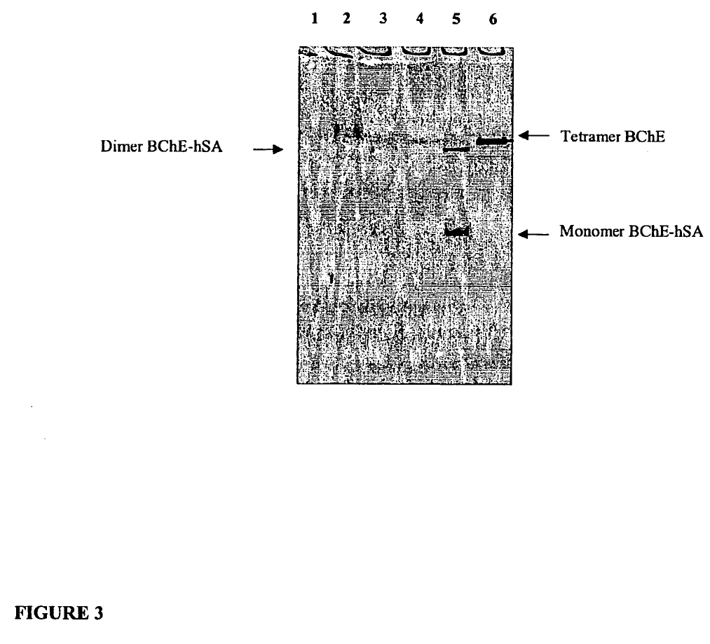

Conditioned media from pCMV/IgKBChE transfected MAC-T cells showed an active protein, migrating at the molecular weight size of a tetramer (FIG. 3, lane 2). Conditioned media from MAC-T cells transfected with pCMV/BChE/hSA also showed expression of an active tetramer, as well as of active monomers and dimers (FIG. 3, lane 3). Conditioned media from BHK cells transfected with pCMV/BChE/hSA showed high level expression of both an active monomer and an active dimer (FIG. 3, lane 5)

The finding that MAC-T cells produce recombinant BChE predominantly in tetramer form is unexpected. In prior reports of recombinant expression of BChE in in vitro cultured cells, the tetrameric form was the least abundant (e.g., Blong, et al. Biochem J. (1997) 327:747-757). Thus, the present invention provides for dramatically improved yields of tetrameric BChE enzyme (at least 50% of the produced BChE enzyme) using MAC-T cells transfected with the expression constructs of the invention.

This result also confirms that the recombinant BChE-hSA fusion protein is catalytically active, and may assemble into the dimeric form.

### Example 2

### Production of Recombinant Human BChE in Transgenic Mice

Expression Construct pBCNN/BChE

In this expression construct, the BChE-encoding sequence is under the transcriptional control of a strong β-casein promoter to direct expression of recombinant BChE in the mammary gland, and linked to a β-casein signal sequence to direct secretion of recombinant BChE into milk produced by the mammary gland.

pUC18/BCNN

The goat β-casein promoter, including sequences through exon 2, were reverse PCR amplified from a genomic DNA library (SphI restriction digest) generated using goat blood (Clontech Genome Walking Library), using primers ACB582 (5′CAG CTA GTA TTC ATG GAA GGG CAA ATG AGG 3′) (SEQ ID NO: 41) and ACB591 (5′ TAG AGG TCA GGG ATG CTG CTA AAC ATT CTG 3′) (SEQ ID NO: 42). The 6.0 kb product was subcloned into the pUC18 vector (Promega) and designated pUC18/5′bCN.

A 4.5 kb DNA fragment spanning exon 7 and the 3′ end of the goat β-casein gene was reverse PCR amplified from the same library (BglI restriction digest) using primers ACB583 (5′ CCA CAG AAT TGA CTG CGA CTG GAA ATA TGG 3′) (SEQ ID NO: 43) and ACB601 (5′ CTC CAT GGG TAA GCC TAA ACA TTG AGA TCT 3′) (SEQ ID NO: 44). The fragment was subcloned in the pUC18 vector as designated pUC18/3′bCN.

The 4.3 kb fragment encompassing exon 7 and the 3′ end of the goat α-casein gene was then PCR amplified from pUC18/3′bCN, using primer ACB620 (5′CTT TCT CAG CCC AAA GTT CTG CCT GTT C₃′) (SEQ ID NO: 45), which introduces NotI and XhoI sites and primer ACB621 (5′CAA GTT CTC TCT CAT CTC CTG CTT CTC A 3′) (SEQ ID NO: 46), which introduces SalI and Not I sites. This fragment was subcloned into the pUC18 vector and designated pUC18bCNA.

A 4.9 kb fragment containing the 5′ end of the β-casein promoter including sequences through exon 2 was PCR amplified from pUC18/5′bCN using primer ACB618 (5′CAG TGG ACA GAG GAA GAG TCA GAG GAA G 3′) (SEQ ID NO: 47), which introduces a BamHI and SacI site at the 5′end and primer ACB619 (5′ GTA TTT ACC TCT CTT GCA AGG GCC AGA G 3′) (SEQ ID NO: 48), which is near the starting ATG codon and introduces a XhoI site. This fragment was then subcloned into the pUC18bCNA expression vector by digesting with XhoI, which digests at the 5′ end of the 3′ bCN fragment and BamHI, which is present in the pUC18 vector just upstream of the XhoI site. This ligation generates the final pUC18/BCNN construct, which contains the β-casein promoter, including sequences upto exon 2, followed by an XhoI site, exon 7 and the 3′ end of the β-casein gene.

pBCNN/BChE

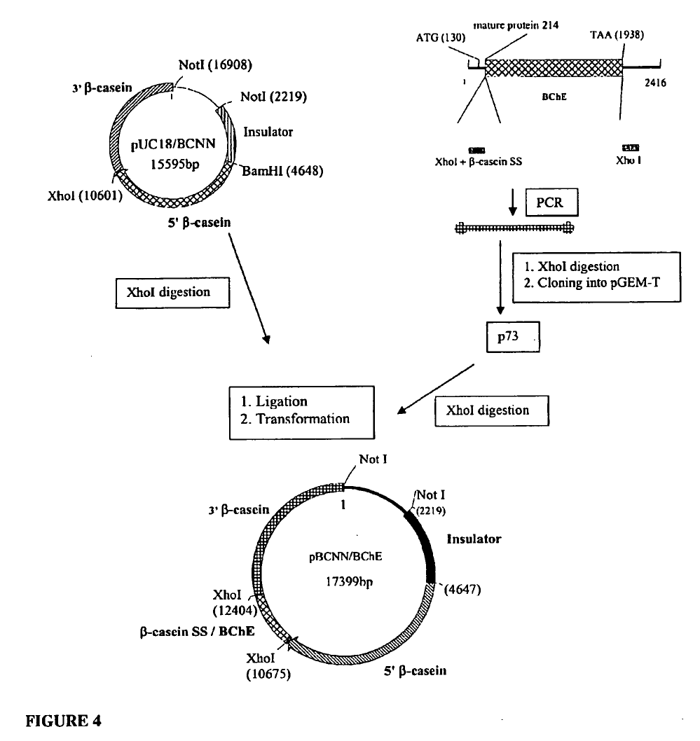

The human BChE cDNA was PCR amplified from a cDNA clone (ATCC #65726) with a sense primer Acb719 (5′ ATA TTC TCG AGA GCC ATG AAG GTC CTC ATC CTT GCC TGT CTG GTG GCT CTG GCC CTT GCA AGA GAA GAT GAC ATC AT 3′) (SEQ ID NO: 9) containing an XhoI restriction endonuclease site (underlined), goat β-casein signal sequence (italic), and a partial human BChE sequence; and an antisense primer, Acb718 (5′CTA TGA CTC GAG GCG ATC GCT ATT AAT TAG AGA CCC ACA C₃′) (SEQ ID NO: 10) containing an XhoI site (underlined) and partial 3′ human BChE sequence. The BChE PCR product was XhoI digested and subcloned into pGEM-T easy vector (Promega), to given the construct named p73. The BChE insert of p73 was excised by digestion with XhoI, purified with GFX matrix (Pharmacia Biotech, Baie d'Urf, PQ, Canada) and ligated with XhoI-digested pUC18/BCNN to generate pBCNN-BChE. The generation of pBCNN/BChE is shown schematically in FIG. 4.

pBCNN/BChE was digested with NotI, and the resultant NotI-digested linear DNA, free of bacterial sequences, was prepared and used to generate transgenic mice. Briefly, circular expression construct DNA was purified by the cesium chloride gradient technique. This purified DNA was restricted with NotI, electrophoresed, and the linear DNA fragment was gel purified. The DNA fragment was then mixed with cesium chloride and centrifuged at 20° C., 60,000 rpm for 16 to 20 hrs in a Beckman L7 ultracentrifuge using a Ti70.1 rotor (Beckman Instruments, Fullerton, Calif., USA). The DNA band was removed, dialyzed against WFI water for 2-4 hrs, and precipitated in ethanol. The precipitated DNA was resuspended in injection buffer (5 mM Tris pH 7.5, 0.1 mM EDTA, 10 mM NaCl) and dialyzed against the same buffer at 4° C. for 8 hrs. Two additional dialysis steps were performed, one for 16 hrs and the second for at least 8 hrs. After dialysis the DNA was quantitated using a fluorometer. Prior to use an aliquot was diluted to 2-3 ng/ml in injection buffer.

As a result of this preparation, the linear BCNN/BChE fragment used to generate transgenic animals contained, in this order:

Dimerized Chicken β-Globin Gene Insulator

Goat Beta-Casein Promoter

β-casein exon 1;

β-casein intron 1;

Partial β-casein exon 2;

XhoI cloning site;

β-casein signal sequence;

BChE-encoding sequence;

A STOP codon;

Partial β-casein exon 7;

β-casein intron 7;

β-casein exon 8;

β-casein intron 8;

β-casein exon 9; and

Additional α-casein 3′ genomic sequence.

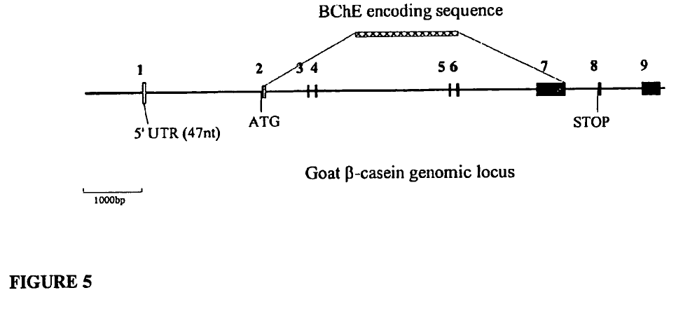

A schematic depicting the exons and introns of the goat β-casein locus that are contained in this fragment is shown in FIG. 5.

2.2. Production of Founders and Subsequent Generations of Transgenic Mice.

The production and maintenance of transgenic mice were conducted at the Mcintyre Transgenic Core Facility of McGill University. Transgenic mice were generated by pronuclear microinjection essentially as described in Hogan, et al. “Manipulating the Mouse Embryo: A Laboratory Manual.” Cold Spring Harbor Laboratory, 1986. The BCNN/BChE linear fragment was microinjected into 414 fertilized eggs (strain FVB) and 22 pups were born.

At 2-3 weeks of age tail biopsies were taken, under anesthesia and DNA was prepared according to standard procedures well known to those skilled in the art, and described in detail, for example, in “Molecular Cloning: A Laboratory Manual.” 2^{nd} Edition Sambrook, et al. Cold Spring Harbor Laboratory:1989). The presence of the transgene in genomic DNA was confirmed by PCR and/or Southern analysis as described in Identification of transgenic mice below. Out of 28 tail DNA samples, 2 dead pup and 4 live founders (2 males and 2 females) were confirmed transgene positive. Southern analysis was also used to estimate transgene copy number.

Transgenic founder mice were bred with wild-type mice of the same strain for the generation of subsequent transgenic generations. One founder female has been used to establish a transgenic line with ˜10 copies of the transgene. The other female and one of the male founders have been used to establish a trasgenic line with ˜40 copies of the transgene. As shown in Table 2, the transgene was stably transmitted for 2 generations.

2.3. Identification of Transgenic Mice.

PCR Analysis:

Genomic DNA purified from tail biopsies was quantitated by fluorimetry and PCR screened using three different primer sets. PCR was performed with the Ready-To-Go™ PCR beads (Pharmacia Biotech). Upon amplification the samples were analysed for the presence of the PCR product by electrophoresis on a 2% agarose gel. The quality of the DNA used in these PCR reactions was confirmed by the presence of the expected fragment of the endogenous mouse β-casein gene.

Primer set A, ACB712 (5′ CTT CCG TGG CCA GAA TGG AT 3′) (SEQ ID NO: 11) and ACB244 (5′ CAT CAG AAG TTA AAC AGC ACA GTT AGT 3′) (SEQ ID NO: 12), amplifies a 495 bp fragment from the 3′ end of the transgene spanning the junction of the BChE and 3′ genomic β-casein sequences.

Primer set B, ACB268 (5′ AGG AGC ACA GTG CTC ATC CAG ATC 3′) (SEQ ID NO: 13) and ACB659 (5′ GAC GCC CCA TCC TCA CTG ACT 3′) (SEQ ID NO: 14), amplifies a 893 bp fragment of the insulator sequence located at the 5′ end of the transgene.

Primer set C, ACB572 (5′ TTC CTA GGA TGT GCT CCA GGC T 3′) (SEQ ID NO: 15) and ACB255 (5′ GAA ACG GAA TGT TGT GGA GTG G 3′) (SEQ ID NO: 16) amplifies a 510 bp portion of an endogenous mouse β-casein gene. This primer set serves as in internal positive control to indicate that the extracted DNA can be amplified by PCR.

Southern Blotting Analysis:

Confirmation of transgene presence, and estimation of transgene copy number, was performed using Southern blotting analysis with Boehringer Mannheim's DIG system. Genomic DNA (5 μg) extracted from tail biopsies was digested with XmeI and ApaLI. This digestion was followed by gel electrophoresis and Southern transfer to nylon membranes (Roche Diagnostics Canada). The blot was hybridized in a DIG Easy Hyb buffer (Roche Diagnostics Canada) at 42° C. overnight using an insulator probe labeled by the PCR DIG probe synthesis kit (Roche Diagnostics Canada), which hybridizes at the 5′ end of the transgene. This insulator probe was PCR amplified from the pBCNN/BChE construct using the primers Acb266 (5′ TGC TCT TTG AGC CTG CAG ACA CCT 3′) (SEQ ID NO: 17) and Acb267 (5′ GGC TGT TCT GAA CGC TGT GAC TTG 3′) (SEQ ID NO: 18). The membrane was washed, detected by the CDP-Star™ substrate (Roche Diagnostics Canada) and visualized by the Fluor Chem™ 8000 System (Alpha Innotech Corporation). The size of the genomic DNA fragment detected by this probe varies depending on the site of integration.

The same membrane was stripped with stripping buffer (Roche Diagnostics Canada) and re-hybridized with a DIG-labeled PCR probe hybridizing within the BChE sequence. The probe was PCR amplified from the pBCNN/BChE construct using the primers Acb710 (5′ GTG TAA CTC TCT TTG GAG AAA G 3′) (SEQ ID NO: 5) and Acb819 (5′CCA GAG GTA AAC CAA AGA C₃′) (SEQ ID NO: 19). This 725 bp BChE-encoding sequence probedetects a 11.kb band of the transgene.

Upon analysis, the expected size bands were detected for all transgenic offspring and copy number was estimated. Transgene copy number has been stable for at least two generations (see Table 3). For example, the founder transgenic male (F0) with ˜40 copies of the transgene has transmitted ˜40 copies to all of his offspring (F1).

2.4. Analysis of Recombinant BChE in the Milk Transgenic Mice

Lactating female mice were milked after induction with an intraperitoneal injection of 5 i.u. of oxytocin.

The milking apparatus is described at www.invitrogen.co-m/ContentITech-online/molecular_biology/manuals_pps/pbc1_man.pdf. The amount of milk that was obtained varied from 50-100 μl. The milk was centrifuged at 3000.times.g for 30 minutes at 4° C., and the resultant whey phase was separated from the fat phase and precipitates. The whey phase was stored at −20° C. until analysis.

he milk was analyzed for BChE activity levels using the Ellman Assay, and for oligomerization of recombinant BChE by analysis on non-denaturing activity gels. It is important to note that mouse milk contains endogenous levels of BChE activity that were controlled for in performing the activity assays. The non-denaturing activity gels showed a unique band for the endogenous mouse BChE that did not co-migrate with the recombinant BChE.

Levels BChE Activity Measured using the Ellman Assay

The Ellman BChE activity assay was performed on the whey phase of milk collected from transgenic mice. The whey phase of milk from 2 wild type FVB mice served as negative controls, while a partially purified human plasma BChE sample served as a standard. Samples were added in 100 μl of 0.1 M potassium phosphate buffer (pH 8.0) into each well of duplicate 96-well plates. 50 μl of DTNB reaction buffer were added into each well, and then mixed well. The plate was incubated at room temperature for 10 minutes. Absorbance of the plate at 405 nm was measured with Vmax Kinetic Microplate Reader (Molecular Devices) with SoftMax™ software and used as baseline reading prior to measuring product formation. 100 μl of S-butyrylthiocholine iodide were pipetted into each well with a multiple pipette and mixed. Absorbance at a wavelenght of 405 nm was measured at 1 min, 5 min and 10 min. One unit was defined as the amount of BChE that hydrolyzed 1 micromol of substrate/min.

A specific activity of 720 Units/mg, measured at 25° C. with 1 mM butyrylthiocholine in 0.1 M potassium phosphate (pH 8.0), was the standard for purified human BChE. The activity detected using the milk of two negative control mice (0.7 Units/ml, 0.97 mg/ml; 0.84 Unites/ml; 1.16 mg/ml) was subtracted from the activity detected in the milk of the transgenic mice. The results (see Table 3) clearly show that BChE activity was detected in both founder trangenic mice (F0 generation) and in the milk of female offspring (F1 generation).

Analysis of Non-Denaturing BChE Activity Gels

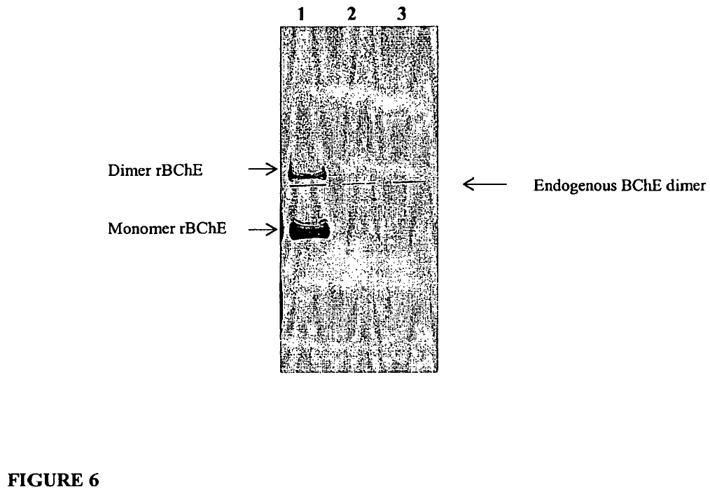

The collected whey phase samples were also electrophoresed on native 4-20% pre-cast TRIS-glycine gels (Invitrogen) at 100 V overnight and 4° C. The gels were then stained for BChE activity with 1 mM of butyrylthiocholine iodide according to the Kamovsky and Roots method (Karnovsky and Roots Histochem. Cytochem. (1964) 12:219-221). The staining procedure was performed at ambient temperature for two to six hours until the active protein bands were revealed. As can be seen from FIG. 6, the endogenous mouse BChE present in milk (lanes 2 and 3) migrates at a different size than the recombinant human BChE (lane 1). The recombinant human BChE is produced as a mixture of dimers and monomers, while the endogenous BChE is predominantly a dimer.

The above results demonstrate that recombinant human BChE can be produced and secreted by the mouse mammary gland, with the resultant milk containing levels of up to greater than 1.5 g/L of recombinant human BChE (see Female 4 in Table 3). The secretion of recombinant BChE has no adverse effects on lactation, as shown by the ability of transgenic females to nurse their pups.

### Example 3

### Production of Recombinant BChE-hSA Fusion Protein in Transgenic Mice

The methods and protocols used for this example, unless otherwise stated, were the same as those used for Example 2.

3.1. Expression Construct pBCNN/BChE/hSA

pBCNN/wtBChE/hSA

The vector pBCNN/BChE (see Example 2.1 and FIG. 4) was digested with XhoI to remove the BChE insert, blunt-ended by filling in with Klenow polymerase in the presence of dNTPs, and CIP treated. Construct pCMV/BChE/hSA (See Example 1.1) was partially digested with NcoI to remove the BChE-hSA encoding sequences, blunt-ended by filling in with Klenow polymerase in the presence of dNTPs, and PmeI digested. The two blunt-ended fragments were ligated to generate pBCNN/wtBChE/hSA. In this construct the signal sequence is the BChE signal sequence.

pBCNN/BChE/hSA

The BstAPI fragment (from 4976 nt to the middle part of BChE) of pBCNN/wtBChE/hSA was replaced with the same BstAPI fragment from pBCNN/BChE (See Example 2.1) to generate pBCNN/BChE/hSA. In this construct the signal sequence is from goat β-casein.

pBCNN/BChE/hSA was digested with NotI, and the resultant NotI-digested linear DNA, free of bacterial sequences, was prepared and used to generate transgenic mice. Briefly, circular expression construct DNA was purified by the cesium chloride gradient technique. This purified DNA was restricted with NotI, electrophoresed, and the linear DNA fragment was gel purified. The DNA fragment was then mixed with cesium chloride and centrifuged at 20° C., 60,000 rpm for 16 to 20 hrs in a Beckman L7 ultracentrifuge using a Ti70.1 rotor (Beckman Instruments, Fullerton, Calif., USA). The DNA band was removed, dialyzed against WFI water for 2-4 hrs, and precipitated in ethanol. The precipitated DNA was resuspended in injection buffer (5 mM Tris pH 7.5, 0.1 mM EDTA, 10 mM NaCl) and dialyzed against the same buffer at 4° C. for 8 hrs. Two additional dialysis steps were performed, one for 16 hrs and the second for at least 8 hrs. After dialysis the DNA was quantitated using a fluorometer. Prior to use an aliquot was diluted to 2-3 ng/ml in injection buffer.

3.2 Production of Founders and Subsequent Generations of BChE/hSA Transgenic Mice

The production and maintenance of transgenic mice were conducted at McIntyre Transgenic Core Facility of McGill University. Transgenic mice were generated by pronuclear microinjection essentially as described in Hogan, et al. “Manipulating the Mouse Embryo: A Laboratory Manual.”Cold Spring Harbor Laboratory, 1986. The BCNN/BChE linear fragment was microinjected into 519 fertilized eggs (strain FVB), and 27 pups were born (see Table 2 for details).

At 2-3 weeks of age tail biopsies were taken under anesthesia and DNA was prepared according to standard procedures well known to those skilled in the art, and described in detail, for example, in “Molecular Cloning: Laboratory Manual.” 2nd Edition. Sambrook, et al. Cold Spring Harbor Laboratory: 1989. The presence of the transgene in the genomic DNA was confirmed by PCR analysis as described in Identification of Transgenic Mice below. Out of 29 tail DNA samples, 1 female founder and one dead pup were confirmed transgene positive.

3.3. Identification of Transgenic Mice.

The presence of the transgene in mice was confirmed by PCR as described in Example 2.3, except that PCR primer set A was replaced with primer set 1, primers ACB712 (5′CTT CCG TGG CCA GAA TGG AT 3′) (SEQ ID NO: 11) and ACB884 (5′CCT CAC TCT TGT GTG CAT CG 3′) (SEQ ID NO: 20), which amplifies a 462 bp fragment from the 3′ end of the transgene spanning the junction of the BChE and albumin sequences.

TABLE 2
Transgenic mice produced via pronuclear microinjection
BCNN-BChE construct

| | |
|---|---|
| Eggs microinjected | 414 |
| Eggs transferred to recipients | 265 |
| Recipient mice (average | 9 (25) |
| embryos per recipient) | |
| % Recipients pregnant | 56% |
| Pups born | 28 |
| Pups transgenic (Male/Female; | 6/28 (2/2, 2 dead; 21%) |
| dead; % transgenic) | |

pBCNN/BChE/hSA

| | |
|---|---|
| Eggs microinjected | 516 |
| Eggs transferred to recipients | 294 |
| Recipient mice (average embryos | 13 (26) |
| per recipient) | |
| % Recipients pregnant | 61% |
| Pups born | 32 |
| Pups transgenic (Male/Female, | 2/27 (0/1, 1; 7%) |
| dead; % transgenic) | |

TABLE 3
Transgene copy number and analysis of BChE activity in milk of
transgenic mice BCNN-BChE

| Founder | Copy | Ellman | F1 | | | Ellman |
|---|---|---|---|---|---|---|
| (F0) bred | # | (mg/L) | transmission | F1 bred | Copy # | (mg/L) |
| Male A. | ˜40 | NA | 14/21 | Male 1 | ˜40 | NA |
| | | | (67%) | Male 2 | ˜40 | NA |
| | | | 6 Males | Male 3 | ˜40 | NA |
| | | | 8 Females | Male 4 | ˜40 | NA |
| | | | | Male 5 | ˜40 | NA |
| | | | | Male 6 | ˜40 | NA |
| | | | | Female 1 | ˜40 | 418 |
| | | | | Female 2 | ˜40 | 151 |
| | | | | Female 3 | ˜40 | 388* |
| | | | | Female 4 | ˜40 | 1800 |
| Female A | ˜10 | 3.5 | ND | ND | ND | ND |
| Female B | ˜40 | 390* | 5/19 | Male 7 | ˜40 | NA |
| | | | (26%) | Male 8 | ˜40 | NA |
| | | | 4 Males | Male 9 | ND | NA |
| | | | 1 Female | Male 10 | ND | NA |
| | | | | Female 5 | ND | 910 |

NA = not applicable.
ND = not done.
*Value represents the average of three independent assays.

3.4. Expression of the Recombinant BChE-hSA Fusion Protein in Transgenic Mice.
Levels BChE Activity Measured using the Ellman Assay

The Ellman BChE activity assay is performed on the the whey phase of milk collected from the female founder mouse (as described in Example 2.4). The activity detected using the milk of two negative control mice is subtracted from the activity detected in the milk of the transgenic mouse. This assay will be used to confirm that the recombinant BChE-hSA fusion is catalytically active.

### Example 4

### Production of Recombinant Human BChE in Transgenic Goats

4.1. Hormonal Treatment of Oocyte Donor Goats:

Recipient and donor crossbreed goats (mainly Saanen.times.Nubian) were estrus synchronized by means of an intravaginal sponge impregnated with 60 mg medroxyprogesterone acetate (Veramix™, Pharmacia Animal Health, Ontario, Canada) for 10 days, together with a luteolytic injection of 125 μg clorprostenol (Estrumate™, Schering, Canada) administered intramuscularly 36 hours prior to sponge removal. In addition, for donor goats follicular development was stimulated by a gonadotrophin treatment consisting of 70 mg NIH-FSH-P1 (Folltropin-V™, Vetrepharm, Canada) and 300 IU eCG (Novormon 5000™, Vetrepharm, Canada) administered intramuscularly 36 h prior to Laparaoscopic Ovum Pick-Up (LOPU).

4.2. Collection of Cumulus Oocyte Complexes (COCs) From Donor Goats by Laparoscopic Ovum Pick-Up (LOPU).

Cumulus oocyte complexes (COCs) from donor goats were recovered by aspiration of follicle contents (puncture or folliculocentesis) under laparoscopic observation. The laparoscopy equipment used (Richard Wolf, Germany) was composed of a 5 mm telescope, a light cable, a light source, a 5.5 mm trocar for the laparoscope, an atraumatic grasping forceps, and two 3.5 mm “second puncture” trocars. The follicle puncture set was composed of a puncture pipette, tubing, a collection tube, and a vacuum pump. The aspiration pipette was made using an acrylic pipette (3.2 mm external diameter, 1.6 mm internal diameter), and a 20G short bevel hypodermic needle, which was cut to a length of 5 mm and fixed into the tip of the pipette with instant glue. The connection tubing was made of clear plastic tubing with an internal diameter of 5 mm, and connected the puncture pipette to the collection tube. The collection tube was a 50 ml centrifuge tube with an inlet and an outlet available in the cap. The inlet was connected to the aspiration pipette, and the outlet was connected to a vacuum line. Vacuum was provided by a vacuum pump connected to the collection tube by means of clear plastic 8 mm tubing. The vacuum pressure was regulated with a flow valve and measured as drops of collection medium per minute entering the collection tube. The vacuum pressure was typically adjusted to 50 to 70 drops per minute.

The complete puncture set was washed and rinsed 10 times with tissue culture quality distilled water before gas sterilization, and one time before use with collection medium, M199+25 mM HEPES (Gibco) supplemented with penicillin, streptomycin, kanamycin, bovine serum albumin and heparin). Approximately 0.5 ml of this medium was added to the collection tube to receive the oocytes.

Donors were deprived of food for 24 hours and of water for 12 hours prior to surgery. The animals were pre-anesthetized by injection of diazepam (0.35 mg/kg body weight) and ketamine (5 mg/kg body weight). Thereafter, anesthesia was maintained by administration of isofluorane via endotrachial intubation. Preventive antibiotics (e.g., oxytetracycline) and analgesic/anti-inflammatorues (e.g., flunixine) were administered by intramuscular injection in the hind limbs. The surgical site was prepared by shaving the abdominal area, then scrubbing first with soap and water and then with a Hibitaine:water solution, followed by application of iodine solution.

A small incision/puncture was made with a scalpel blade about 2 cm cranial from the udder and about 2 cm left from the midline. The 5 mm trocar was inserted and the abdominal cavity was inflated with filtered air through the trocar sleeve gas valve. The laparoscope was inserted into the trocar sleeve. A second incision was made about 2 cm cranial from the udder and about 2 cm right from the midline, into which was inserted a 3.5 mm trocar. The trocar was removed, and the forceps was inserted. A third incision was made about 6 cm cranial to the udder and about 2 cm right from the midline. The second 3.5 mm trocar and trocar sleeve was inserted into this incision. The trocar was removed and the aspiration pipette connected to the vacuum pump and the collection tube was inserted therein.

After locating the reproductive tract below the bladder, the ovary was exposed by pulling the fimbria in different directions, and the number of follicles available for aspiration was determined. Generally, follicles greater than 2 cm were considered eligible for aspiration. The follicles were punctured one by one and the contents aspirated into the collection tube under vacuum. The needle was inserted into the follicle and rotated gently to ensure that as much of the follicle contents as possible were aspirated. After >10 follicles were aspirated and/or before moving to the other ovary, the pipette and tubing were rinsed using collection media from a sterile tube.

4.3. In Vitro Maturation of Oocytes Collected by LOPU

To each collection tube containing cumulus oocyte complexes (COCs) was added about 10 ml of searching medium, EmCare™ supplemented with 1% heat inactivated Fetal Bovine Serum (FBS). The resulting solution was aspirated into a grid search plate and transferred to Petri dishes containing the same medium for the purpose of scoring each COC for amount and expansion of cumulus. The COCs were then washed with in vitro maturation (IVM) medium; (M199+25 mM HEPES supplemented with bLH, bFSH, estradiol β-17, pyruvate, kanamycin and heat-inactivated EGS) that had been equilibrated in an incubator under 5% CO₂ at 35.5° C. for at least 2 hours. The COCs were pooled in groups of 15-25 per droplet of IVM medium, overlayed with mineral oil, and incubated in 5% CO.sub.2 at 35.5° C. for 26 hours.

4.4. Preparation of Semen for In Vitro Fertilization

Fresh semen was collected from 2 adult Saanen males of known fertility. After collection, sperm capacitation was achieved as follows. A 5 μl aliquot of fresh semen was diluted in 500 μl warm modified Defined Medium (mDM) comprising NaCl, KCl, NaH.sub.2PO₄H₂, MgCl₆H₂O, CaCl₂.2H₂O, glucose, 0.5% phenol red, Na-Pyruvate, NaHCO.sub.3, gentamicin and BSA. The solution was allowed to stand at room temperature in the absence of light for 3 hours. An additional 1 ml of mDM solution was added and 100 μl of the resulting solution was overlaid on a 45%:90% Percoll gradient [Percoll (Sigma P1644) in modified Sperm Tyrodes Lactate (SPTL) solution] in a conical centrifuge tube. The solution was centrifuged on the Percoll gradient at 857.times.g for 30 minutes. The pellet was resuspended in mDM solution and centrifuged at the same speed for 10 minutes. The pellet was re-suspended in capacitation medium (mDM, supplemented with 8b-cAMP, lonomycin and Heparin). The resuspended semen was cultured at 38.5° C. under 5% CO₂ for 15 minutes. The sperm concentration was then adjusted to final concentration of 20×10⁶ sperm/ml by addition of mDM solution.

4.5 In Vitro Fertilization of Oocytes

The expanded cumulus cells were partially removed from the matured COCs by pipetting repeatedly through two fine-bore glass pipettes (200 and 250 μm internal diameter), leaving one layer of cumulus cells on the zona. The oocytes were washed with in vitro fertilization (IVF) medium, a modified Tyrode's albumin lactate pyruvate (TALP), and transferred to 40 μl droplets of the same medium (15-20 oocytes per 40 μl droplet) under mineral oil. A 5 μl aliquot of the capacitated sperm suspension (20×10⁶ sperm/ml), prepared as described in Example 4.4, was added to each 40 μl droplet. The inseminated oocytes were cultured at 38.5° C. in 5% CO₂ for 15-16 hours.

4.6 Pronuclear Microiniection of Oocytes

After culturing for 15-16 hours, the cumulus cells were stripped from the inseminated oocytes (zygotes) by repeated pipetting as described above. The zygotes were then observed for pronuclear formation using an Olympus stereomicroscope. To improve pronucleus visualization, the zygotes were washed in EmCare™ (PETS, cat. # ECFS-100) supplemented with 1% Fetal Bovine Serum (FBS), (Gibco BRL, Australian or New Zealand sourced, heat inactivated at 56° C. for 30 minutes), then centrifuged at 10,400.times.g for 3 minutes before observation. Zygotes with visible pronuclei were selected for microinjection and transferred to 50 μl droplets of temporary culture medium (INRA Menezo B2, Meditech cat. #CH-B 04001 supplemented with 2.5% FBS) during manipulation. The zygotes were then transferred to 50 μl droplets of EmCare™+1% FBS (about 20 zygotes per droplet) and microinjected with the BCNN/BChE linear fragment from Example 2.1. (3 ng/ml of the DNA in a buffer of 5 mM Tris, 0.1 mM EDTA, 10 mM NaCl buffer, pH 7.5). The injected zygotes were washed and cultured in temporary culture medium to await transfer to recipients.

4.7 Transfer of Embrvos to Oviduct Recipient Goats and Birth of Kids

Adult goats of various breeds including the Boer, Saanen, and Nubian breeds were used as recipients. They were estrus synchronized by means of an intravaginal sponge impregnated with 60 mg medroxyprogesterone acetate (Veramix™, Pharmacia Animal Health, Ontario, Canada) left in place for 9 days, together with a luteolytic injection of 125 μg clorprostenol (Estrumate™, Schering, Canada) and 500 IU eCG (Novormon 5000™, Vetrepharm, Canada) administered intramuscularly 36 hours prior to sponge removal. Sponges were inserted into the recipient goats on the same day as the donor goats but removed approximately 15 hours earlier. Each recipient was subsequently treated with an intramuscular injection of 100 μg GnRH (Factrel™, 2.0 ml of 50 μg/ml solution), 36 hours after sponge removal. The recipients were tested for estrus with a vasectomized buck at 12 hour intervals beginning 24 hours after sponge removal and ending 60-72 hours after sponge removal.

Recipient goats were fasted, anesthetized, and prepared for surgery following the same procedures previously described for donor goats. They also received preventive antibiotic therapy and analgesic/anti-inflammatory therapy, as described for donors. Prior to surgery, a laparoscopic exploration of each eligible recipient was performed to confirm that the recipient had one or more recent ovulations (as determined by the presence of corpora lutea on the ovary), and a normal oviduct and uterus. The laparoscopic exploration was carried out to avoid performing a laparotomy on an animal which had not responded properly to the hormonal synchronization protocol described above. Two incisions were made (one 2 cm cranial to the udder and 2 cm left of the midline, and the other 2 cm cranial to the udder and 2 cm right of the midline) and the laparoscope and forceps were inserted as described above. The ovaries were exposed by pulling up the fimbria with the forceps, and the number of ovulations present as well as the number of follicles larger than about 5 mm diameter was noted. Recipients with at least one ovulation present and having a normal uterus and oviduct were eligible for transfer. A mid-ventral laparotomy incision of approximately 10 cm length was established in eligible recipients, the reproductive tract was exteriorized, and the embryos were implanted into the oviduct ipsilateral to the ovulation(s) by means of a TomCat™ catheter threaded into the oviduct from the fimbria. The incisions were closed and the animal was allowed to recover in a post-op room for 3 days before being returned to the pens. Skin sutures were removed 7-10 days after surgery.

Recipients were scanned by transrectal ultrasonography using a 7.5 Mhz linear array probe to diagnose pregnancy at 28 and 60 days after transfer.

Newborn kids were removed from does at birth to prevent disease transmission from doe to kid by ingestion of doe's raw colostrum and/or milk, exposure to doe's fecal matter or other potential sources of disease. Kids were fed thermorized colstrum for the first 48 hours of life, and pasteurized doe milk thereafter until weaning.

4.8. Identification of Transgenic Goats

Blood and tissue samples were taken from putative transgenic kids at approximately 4 days after birth, and again at approximately two weeks after birth. At each sampling interval, about 2-7 ml blood sample was collected from each kid into an EDTA vacutainer, and stored at 4° C. for up to 24 hours until use. Tissue samples were obtained by clipping the ear tip of each kid, and stored at 20° C. until use. Genomic DNA was isolated from the blood samples using a QIAamp DNA Blood Mini Kit (Qiagen, Cat. # 51106), and from the tissue samples using DNeasy Tissue Kit (Qiagen, cat #69506). For each sample, the DNA was eluted in 150-200 μl 0.1.times. buffer AE and stored at 4° C. until ready to use.

PCR screening was performed on each DNA sample to determine the presence of the BChE-encoding transgene. Genomic DNA samples were diluted using nuclease-free water to a concentration of 5 ng/μl. A 20 μl portion of the diluted DNA was added to a 0.2 ml Ready-To-Go PCR tube containing a PCR bead, together with 5 μl 5×primer mix containing dUPT (Amersham Bioscience, cat. #272040) and UDG (Invitrogen, cat. #18054-015). The primer sets used were identical to the ones used in the PCR analysis of Example 2.3, except for primer set C. In this case, primer set C was replaced with the primers Acb256 (5′ GAG GAA CAA CAG CAA ACA GAG 3′) (SEQ ID NO: 21) and Acb312 (5′ ACC CTA CTG TCT TTC ATC AGC 3′) (SEQ ID NO: 22), which amplify a 360 bp portion of the endogenous goat b-casein gene. This primer set serves as in internal positive control to indicate that the extracted DNA can be amplified by PCR.

The sample was subjected to thermal cycling and then applied to a 1% agarose gel. Negative controls (genomic DNA isolated from non-transgenic animals) and positive controls (genomic DNA from non-transgenic animals spiked with the microinjected BCNN/BChE linear fragment) were also included. Samples which exhibited a band corresponding to the positive control were deemed positive. Based on this PCR analysis, a total of 6 transgenic goats were identified (5 females and 1 male).

The presence of the transgene was confirmed by Southern blotting as described in Example 2.3. The expected size bands were detected for all transgenic founders (F0 generation), and transgene copy number was estimated to be between about 4-50 copies (see Table 5). Fluorescent in situ hybridization (FISH) was performed as described in Keefer, et al. Biol. Reprod. (2001) 64:849-856 in order to determine the number of chromosomal integration sites (Table 5).

TABLE 4
Transgenic goats produced via nuclear proinjection

| | |
|---|---|
| Donor goats aspirated | 68 |
| Follicles aspirated (ave. per donor goat) | 1410 (20.7) |
| Oocytes recovered (ave. per donor goat, | 1256 (18.5, 89%) |
| recovery rate) | |
| Zygotes microinjected (% of oocytes recovered) | 724 (58%) |
| Zygotes transferred (% of microinjected) | 635 (88%) |
| Recipient goats (ave. embryos per recipient) | 92 (6.9) |
| Recipients pregnant at 28 days (% pregnant) | 48 (52%) |
| Kids born (ave. per recipient) | 61 (1.7) |
| Kids transgenic (Male/Female; % of kids born) | 6 (5/1; 10%) |

TABLE 5
Trausgene copy number and chromosomal integration sites of founder
transgenic goats.

| Founder goat | Transgene | Integration sites |
|---|---|---|
| (F0 generation) | copy number | (by FISH) |
| Male 1 | ˜5-10 | 3 |
| Female 1 | ˜2-5 | 2 |
| Female 2 | ˜2-5 | 2-3 |
| Female 3 | ˜20 | 1-2 |
| Female 4 | ˜5-10 | 2-3 |
| Female 5 | ND | 1 |

ND = not done

4.9. Induction of Lactation

Female founders were induced to lactate at 3-4 months of age in order to confirm the expression of recombinant BChE in milk. For such purpose they were hormonally stimulated with Estradiol cypionate (0.25 mg/KBW) and Progesterone (0.75 mg/KBW) every 48 h for two weeks, followed by treatment with dexamethasone (8 mg/goat/day) for 3 days. In general, milk production started during the dexamethasone treatment and the animals were milked twice per day for as long as necessary to produce enough material for further testing.

4.10. Analysis of BChE-Activity in the Milk of Transgenic Goats

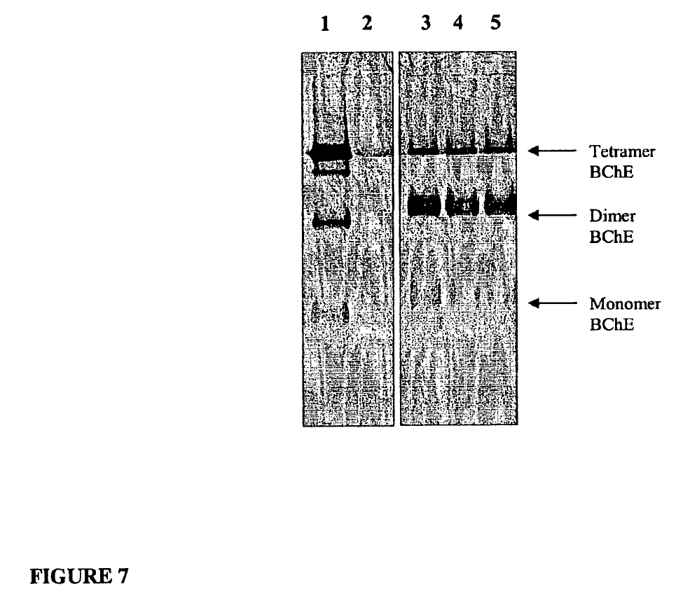

The presence and activity of recombinant BChE in the milk of transgenic goats was analyzed by non-denaturing BChE-activity gel as described in Example 2.4. Such analysis (see FIG. 7) showed that active recombinant BChE is produced in the milk of transgenic goats. The recombinant BChE is present in both a tetramer and dimer form, and to a lesser extent in the monomer form.

4.11. Purification of Recombinant BChE from the Milk of Transgenic Goats Clarification of Milk

20 ml of milk containing recombinant BchE was diluted to 60 ml with 20 mM phosphate buffer (pH7.4). Ammonium sulfate (15 grams) was slowly added to the diluted milk, and the mixture was agitated until all ammonium sulfate solids were dissolved. This liquid was incubated at 4° C. for one hour, and then phase separated by centrifugation at 20,000.times.g for 30 min. The liquid phase containing recombinant BChE was harvested and then dialyzed overnight against 20 mM phosphate buffer (pH7.4), 100 mM sodium chloride, and 1 mM EDTA. 75 ml of liquid containing recombinant BChE was recovered and further clarified by filtration using a 0.2 μm filter. The recovery of BchE based on activity (Ellman reaction) was 50%.

Affinity Chromatography with Procainamide

An affinity resin was prepared using standard protocols with Procainamide (Sigma) and Activated CH Sepharose (Amersham). A column was packed with 20 ml Procainamide affinity resin and equilibrated with 20 mM phosphate buffer (pH7.4), 100 mM sodium chloride, and 1 mM EDTA. The 75 ml of liquid containing recombinant BChE was loaded onto the column at a linear flow rate of 50 cm/hr. The column was washed with 20 mM phosphate buffer (pH7.4), 150 mM sodium chloride, and 1 mM EDTA. BChE was eluted with 20 mM phosphate buffer (pH7.4), 500 mM sodium chloride, and 1 mM EDTA. The eluent containing recombinant BChE was dialysed against 20 mM phosphate buffer (pH7.4), 50 mM sodium chloride, and 1 mM EDTA. A total of 50 ml of liquid containing recombinant BChE was recovered after dialysis. The recovery of BchE after this step was 90%.

Anion Exchange Chromatography

A column was packed with 20 ml HQ50 resin (Applied Biosystems) and equilibrated with 20 mM phosphate buffer (pH7.4), 50 mM sodium chloride, and 1 mM EDTA. The 50 ml of liquid containing recombinant BChE was recovered after affinity chromatography was loaded onto the column at a linear flow rate of 100 cm/h. The column was washed with 20 mM phosphate buffer (pH7.4), 50 mM sodium chloride, and 1 mM EDTA. Purified recombinant BChE was eluted with 20 mM phosphate buffer (pH7.4), 250 mM sodium chloride, and 1 mM EDTA. This eluent was dialyzed against 20 mM phosphate buffer (pH7.4), 100 mM sodium chloride, and 1 mM EDTA, and then further concentrated to a final purfied concentration of 15 mg/ml of protein. The recovery of BChE after this step was 90%.

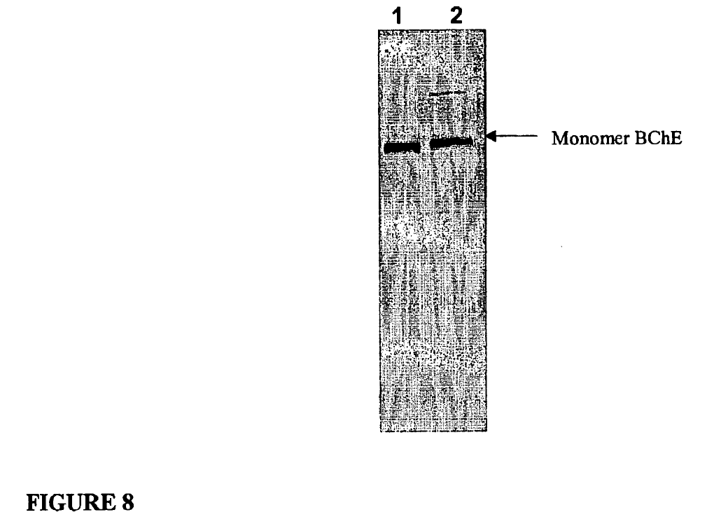

In order to estimate the purity of the purified recombinant BChE, a 0.2 μg sample was subjected to denaturing SDS-PAGE electrophoresis under reducing conditions. The gel was then silver stained to show total protein of the sample (see FIG. 8). Note that all of the purified recombinant BChE migrates as a monomer on this gel, due to reduction of the protein samples with beta-mercaptoethanol prior to loading on the gel, and to denaturation of the proteins during electrophoresis. This analysis was used to estimate that the purified recombinant BchE is >80% pure (compare band intensity of the 0.2 μg sample versus that of 0.2 μg of the positive control).

### Example 5

### Production of Recombinant BChE-hSA Fusion Protein in Transgenic Goats

Trangenic goats expressing a recombinant BChE-hSA fusion protein may be generated by nuclear transfer. The nuclear donors are primary fetal goat cells stably transfected with the BCNN/BChE/hSA linear fragment (from Example 3.1).

5.1. Generation of Stably Transfected Cell Lines

Primary fetal goat cells were derived from day 28 kinder fetuses recovered from a pregnant Saanen breed female goat, and cultured for 3 days prior to being cyropreserved. Chromosome number (2n=60) and sex analysis was performed prior to use of cells for transfection experiments. Under the culture conditions used, all primary lines had a normal chromosome count indicating the absence of gross chromosomal instability during culture.

Transfections were performed as described in Keefer, et al. Biol. Reprod. (2001) 64:849-856, with the following modifications: Female primary lines were thawed and at passage 2, co-transfected with the linearized BCNN/BChE/hSA fragment and the linearized pSV40/Neo selectable marker construct (Invitrogen). The pSV40/Neo linear fragment was generated by restriction of the vector with XbaI and NheI, followed by purification of the fragment as described in Example 2.1. Stably transfected cell lines were selected with G418 and frozen by day 21 (day 0=transfection date).

Four stably transfected cell lines have been derived by this procedure. In all cases the presence of the transgene has been confirmed by Southern Analysis and by Fluorescence In Situ Hybridization (FISH). Transfected cell lines for which integration of the transgene is confirmed will serve as donors for nuclear transfer.

5.2. Oocyte Donor and Recipient Goats

Intravaginal sponges containing 60 mg of medroxyprogesterone acetate (Veramix) are inserted into the vagina of donor goats (Alpine, Saanen, and Boer cross bred goats) and left in place for 10 days. An injection of 125 μg cloprostenol is given 36 h before sponge removal. Priming of the ovaries is achieved by the use of gonadotrophin preparations, including FSH and eCG. One dose equivalent to 70 mg NIH-FSH-P1 of Ovagen is given together with 400 IU of eCG (Equinex) 36 h before LOPU (Laparoscopic Oocyte Pick-Up).

Recipients are synchronized using intravaginal sponges as described above for donor animals. Sponges are removed on day 10 and an injection of 400 IU of eCG is given. Estrus is observed 24-48 h after sponge removal and embryos are transferred 65-70 h after sponge removal.

5.3. Laparoscopic Oocyte Pick-Up (LOPU) and Embryo Transfer

These procedures are performed essentially as described in Examples 4.2 and 4.7

Donor goats are fasted 24 hours prior to laparoscopy. Anesthesia is induced with intravenous administration of diazepam (0.35 mg/kg body weight) and ketamine (5 mg/kg body weight), and is maintained with isofluorane via endotrachial intubation. Cumulus-oocyte-complexes (COCs) are recovered by aspiration of follicular contents under laparoscopic observation.

Recipient goats are fasted and anaesthetized in the same manner as the donors. A laparoscopic exploration is performed to confirm if the recipient has had one or more recent ovulations or corpora lutea present on the ovaries. An average of 11 nuclear transfer-derived embryos (1-cell to 4-cell stage) are transferred by means of a TomCat™ catheter threaded into the oviduct ipsilateral to ovulation(s). Donors and recipients are monitored following surgical procedures and antibiotics and analgesics are administered according to approved procedures.

5.4. Oocyte Maturation

COCs are cultured in 50 μl drops of maturation medium covered with an overlay of mineral oil and incubated at 38.5-39° C. in 5% CO2. The maturation medium consists of M199H (GIBCO) supplemented with bLH, bFSH, estradiol β-17, sodium pyruvate, kanamycin, cysteamine, and heat inactivated goat serum. After 23 to 24 hrs of maturation, the cumulus cells are removed from the matured oocytes by vortexing the COCs for 1-2 min in EmCare™ containing hyaluronidase. The denuded oocytes are washed in handling medium (EmCare™ supplemented with BSA) and returned to maturation medium. The enucleation process is initiated within 2 hr of oocyte denuding. Prior to enucleation, the oocytes are incubated in Hoechst 33342 handling medium for 20-30 minutes at 30-33° C. in air atmosphere.

5.5. Nuclear Transfer

Oocytes are placed into manipulation drops (EmCare™ supplemented with FBS) covered with an overlay of mineral oil. Oocytes stained with Hoechst are enucleated during a brief exposure of the cytoplasm to UV light (Zeiss Filter Set 01) to determine the location of the chromosomes. Stage of nuclear maturation is observed and recorded during the enucleation process.

The enucleated oocytes and dispersed donor cells are manipulated in handling medium. Transgenic donor cells are obtained following either in vitro transfection (see Example 5.1) or biopsy of a transgenic goat. Donor cells are prepared by serum starving for 4 days at confluency. Subsequently they are trypsinized, rinsed once, and resuspended in Emcore™ with serum. Small (<20 μm) donor cells with smooth plasma membranes are picked up with a manipulation pipette and slipped into perivitelline space of the enucleated oocyte. Cell-cytoplast couplets are fused immediately after cell transfer. Couplets are manually aligned between the electrodes of a 500 μm gap fusion chamber (BTX, San Diego, Calif.) overlaid with sorbitol fusion medium. A brief fusion pulse is administered by a BTX Electrocell Manipulator 200. After the couplets have been exposed to the fusion pulse, they are placed into 25 μl drops of medium overlaid with mineral oil. Fused couplets are incubated at 38.5-39° C. After 1 hr, couplets are observed for fusion. Couplets that have not fused are administered a second fusion pulse.

5.6. Oocyte Activation and Culture

Two to three hours after application of the first fusion pulse, the fused couplets are activated using calcium ionomycin and 6-dimethylaminopurine (DMAP) or using calcium ionomycin and cycloheximide/cytochalasin B treatment. Briefly, couplets are incubated for 5 minutes in EmCare™ containing calcium ionomycin, and then for 5 minutes in EmCare™ containing BSA. The activated couplets are cultured for 2.5 to 4 hrs in DMAP, then washed in handling medium and placed into culture drops (25 μl in volume) consisting of G1 medium supplemented with BSA under an oil overlay. Alternately, following calcium ionomycin treatment, the activated couplets are cultured for 5 hrs in cycloheximide and cytochalasin B, washed, and placed into culture. Embryos are cultured 12 to 18 hr until embryo transfer. Nuclear transfer derived embryos are transfered on Day 1 (Day 0=day of fusion) into synchronized recipients on Day 1 of their cycle (D0=estrus).

5.7. Identification of Stably Transfected Cell Lines and of Transgenic Goats

Following selection of transfected cell lines, genomic DNA is isolated from cell pellets using the DNeasy Tissue Kit (Qiagen, cat #69506). For each sample, the DNA is eluted in 150-200 μl 0.1× buffer AE and stored at 4° C. until ready to use.

For confirmation of the presence of the transgene in nuclear transfer derived offspring, genomic DNA is extracted from the blood and ear biopsy of 2 week old kids using standard molecular biology techniques. The genomic DNA is isolated from the blood samples using a QIAamp DNA Blood Mini Kit (Qiagen, Cat. # 51106), and from the tissue samples using DNeasy Tissue Kit (Qiagen, cat #69506). For each sample, the DNA is eluted in 150-200 μl 0.1.times. buffer AE and stored at 4° C. until use.

The presence of the transgene, in stably transfected cells and in transgenic goats, is confirmed by PCR as described in Example 2.3, except for the following modifications. PCR primer set A is replaced with primer set I: Primers ACB712 (5′CTT CCG TGG CCA GAA TGG AT 3′) (SEQ ID NO: 11) and ACB884 (5′CCT CAC TCT TGT GTG CAT CG 3′) (SEQ ID NO: 20) which amplify a 462 bp fragment from the 3′ end of the transgene spanning the junction of the BChE and albumin sequences. Primer set C is replaced with the primers Acb256 (5′ GAG GAA CAA CAG CAA ACA GAG 3′) (SEQ ID NO: 21) and Acb312 (5′ ACC CTA CTG TCT TTC ATC AGC 3′) (SEQ ID NO: 22), which amplify a 360 bp portion of the endogenous goat β-casein gene. This primer set serves as in internal positive control to indicate that the extracted DNA can be amplified by PCR.

The presence of the transgene, in stably transfected cells and in transgenic goats, is also confirmed by Southern blotting as described in Example 2.3. Fluorescent in situ hybridization (FISH) is performed as described in Keefer, et al. Biol. Reprod. (2001) 64:849-856 in order to determine the number of chromosomal integration sites. The FISH probe contains only sequences from the insulator region of the transgene.

### Example 6

Pharmacokinentic Studies of Recombinant BChE Produced by Transgenic Mammals

Residence time of recombinant BChE in the circulation of guinea pigs is determined as described by Raveh, et al. Biochemical Pharmacolocy (1993) 42:2465-2474. A sample BchE enzyme, isolated from the milk of transgenic mammal, is dialyzed against sterile phosphate-buffered saline, pH 7.4. The dialyzed enzyme (50-500 units in a volume of about 250 μl) is administered intravenously into the tail vein of guinea pigs. The injection doses are chosen to be sufficient to provide a plasma concentration of recombinant BChE well above the level of endogenous BChE, as estimated by the Elman assay. At various time intervals, heparinized blood samples (5-10 μl) are withdrawn from the retro-orbital sinus or the toe of the animals and diluted 15 to 20-fold in distilled water at 4 μC. The BchE activity in the blood sample is determined using butyrylthiocholine as the substrate for BChE using the assay of Ellman, et al. (1961). Endogenous ChE activity is subtracted from the result. The clearance of recombinant BchE from the circulation is calculated over time.

To test the efficacy of recombinant BChE in prevention of organophosphate poisoning, nerve agents (soman, VX or sarin or GF) are administered intravenously into the tail vein of guinea pigs in a volume of 100 ul PBS. Animals are observed for 24 hours, and the degree of organophosphate poisoning symptomology recorded. Specifically, percent survival is calculated. Blood sampls are also taken at 10-20 min post nerve agent injection and assayed for residual BchE activity. The level of BChE activity following administration of a nerve agent is a measure of the potency of the recombinant BChE.

### Example 7

### BChE Expression Constructs Based on the WAP Promoter

7.1. Introduction

Whey acidic protein (WAP), the major whey protein in mammals, is expressed at high levels exclusively in the mammary gland during late pregnancy and lactation. The genomic locus of the murine WAP gene consists of 4.4 kb of 5′ flanking promoter sequence, 2.6 kb of coding genomic sequence, and 1.6 kb of 3′ flanking genomic DNA. The WAP promoter may be used to drive expression of heterologous proteins in the mammary gland of transgenic mammals [Velander, et al. Proc. Natl. Acad. Sci. USA (1992) 89: 12003-12007].

An expression construct based on the whey acidic protein (WAP) promoter, can be used to preferentially express BChE in milk of transgenic animals. In one embodiment, the construct is assembled by inserting a BChE-encoding sequence between the WAP promoter (position −949 to +33 nt) at the 5′ end, and the WAP coding genomic sequence (843 bp; the last 30 base of Exon 3, all of intron 3, and exon 4 including 70 bp of 3′ UTR) at the 3′ end. The expression construct also includes two copies of an insulator element from the chicken globin locus. The BChE-encoding sequence may contain the BChE signal sequence or the WAP signal sequence. The BChE-encoding sequence may also contain an epitope tag (e.g., myc and/or his).

In one embodiment, the contruct comprises the WAP gene promoter, the WAP signal sequence, a BChE-encoding sequence, and the coding and 3′ genomic sequences of the WAP gene. This WAP signal sequence is added using a nucleic acid sequence encoding part of the 5′ untranslated region and the 19 amino acid signal peptide of the murine WAP gene (position −949 to +89, Hennighausen, et al. Nucl. Acids Res. (1982) 10:3733-3744). The BChE encoding fragment is generated by PCR of a BChE cDNA (e.g., ATCC #65726) using a 5′ primer containing the 90 bp sequence signal sequence flanked by a KpnI restriction endonuclease recognition site, and 3′ primers containing a KpnI restriction endonuclease recognition site and 3′ BChE cDNA sequences. The amplification is performed to maintain the correct reading frame. This PCR product is then inserted at the KpnI site at the first exon of WAP. The vector is prepared for microinjection or transfection by digestion with NotI restriction endonuclease and purification of the linear fragment.

7.2. Generation of the Expression Construct pWAP/BChE

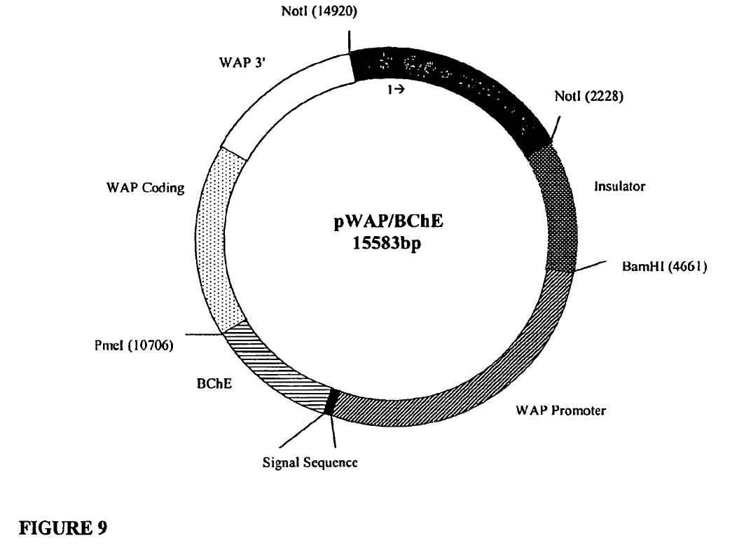

The expression contruct pWAP/BChE (see FIG. 9) may be prepared as follows:

Step 1: PCR Amplification of WAP 3′ Genomic Sequences

The WAP 3′ genomic sequence is PCR amplified from mouse genomic DNA with the following primers: WAP-p1 (5′ MT TGG TAC CAG CGG CCG CTC TAG AGG AAC TGA AGC AGA GAC CAT GC 3′) (SEQ ID NO: 23) and WAP-p2 (5′ GCT GCT CGA GCT TGA TGT TTA AAC TGA TAA CCC TTC AGT GAG CAG CCG ATA TAT GTT TAA ACA TGC GTT GCC TCA TCA GCC TTG TTC 3′) (SEQ ID NO: 24). The PCR product is then restricted with XhoI and NotI.

Step 2: PCR Amplification of WAP Coding Genomic Sequences

The WAP coding genomic sequence (2630 bp) is PCR amplified from mouse DNA with the primers WAP-p3 (5′ ATA TAT GTT TAA ACA TGC GTT GCC TCA TCA GCC TTG TTC 3′) (SEQ ID NO: 25) and WAP-p4 (5′ ATG TTC TCT CTG GAT CCA GGA GTG AAG G 3′) (SEQ ID NO: 26). The PCR product is then restricted with PmeI and BamHI.

Step 3: PCR Amplification of the BChE Encoding Sequence

The BChE encoding sequence (2370 bp) is PCR amplified from a pBChE cDNA with the primers: BChE-p1 (5′ ATT TCC CCG AAG TAT TAC 3′) (SEQ ID NO: 27) and BChE-p2 (5′ TGA TTT TCT GTG GTT ATT 3′) (SEQ ID NO: 28). The PCR product is then blunt ended.

Step 4: Ligation of the WAP Coding and 3′ Genomic Sequences with the BChE Encoding Sequence

The pBluescript vector is restricted with KpnI and Sac II. A linker formed by annealing of the primer sequences Linker-p1 (5′ GGA CCG GTG TTA ACG ATA TCT CTA GAG CGG CCG CT 3′) (SEQ ID NO: 29) and Linker-p2 (5′CCG GAG CGG CCG CTC TAG AGA TAT CGT TAA CAC CGG TCC GC 3′) (SEQ ID NO: 30) is inserted to generate additional restriction enzyme sites (KpnI, NotI, XbaI, EcoRV, HpaI, AgeI and SacI). The new vector is recircularized and then then restricted with EcoRV. The BChE encoding PCR product of Step 3 is then blunt-ended, and ligated to this vector.

This new construct is restricted with XhoI and NotI, and the WAP 3′ genomic sequence PCR product from Step 1 is inserted. This construct is then restricted with PmeI and BamHI and the 2.6 kb WAP coding genomic sequence PCR product of Step 2 is inserted, to generate a construct wherein the BChE-encoding sequence was linked at its 3′ end to the WAP coding and 3′ genomic sequences.

Step 5: PCR Amplification of the Chicken β-Globin Insulator Sequence

The insulator fragment is derived from PCR amplification of chicken genomic DNA with the primers Insulator-p1 (5′ TTT TGC GGC CGC TCT AGA CTC GAG GGG ACA GCC CCC CCC CAA AG 3′) (SEQ ID NO: 31) and Insulator-p2 (5′ TTT TGG ATC CGT CGA CGC CCC ATC CTC ACT GAC TCC GTC CTG GAG TTG 3′) (SEQ ID NO: 32). The PCR product is restricted in two independent reactions; one with NotI and XhoI, and one with BamHI and SalI. The two restricted fragments are then ligated together to generate a 2 kb dimerized insulator fragment with NotI and BamHI sites on either end.

Step 6: Ligation of the WAP Promoter Sequence with the Insulator Fragment

A pBluescript clone containing the 4.4 kb WAP promoter in the pBluescript plasmid [clone 483, described in Velander, et al. Proc. Natl. Acad. Sci. USA (1992) 89:12003-12007] is restricted with SaclI and Not I. A linker formed by annealing of the primer sequences Linker-p3 (5′ GGA CTA GTT GAT CAG CGG CCG CTA TAG GAT CC 3′) (SEQ ID NO: 33) and Linker-p4 (5′GGC CTG GAT CCT ATA GCG GCC GCT GAT CAA CTA GTC CGC 3′) (SEQ ID NO: 34) is inserted to generate a recircularized construct of the 4.4 kb WAP promoter containing additional restriction sites (SaclI, SpeI, BclI, NotI and BamHI). This new construct is then restricted with Not I and BamHI and ligated to the insulator fragment from Step 5.

Step 7: Generation of pWAP/BChE

The BChE/WAP coding and 3′ genomic sequence construct from Step 4 is then restricted with SaclI and AgeI. The 6.8 kb fragment containing the insulator and WAP promoter is isolated from the construct of Step 6 by restriction with SaclI and AgeI. These two fragments are ligated to form pWAP/BChE. This final construct contains the dimerized chicken α-globin gene insulator followed by the WAP 4.4 kb promoter, the BChE gene, and the WAP 2.6 kb coding and 1.6 kb 3′ genomic sequences (See FIG. 9).

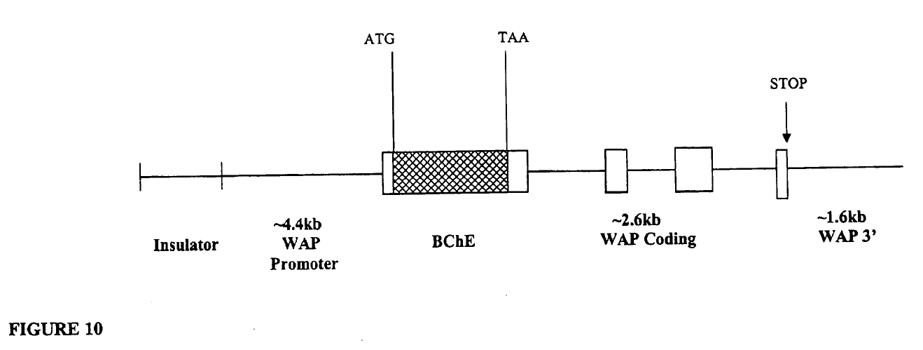

For microinjection or transfection, pWAP/BChE is linearized by NotI digestion to remove the vector sequences. This linearized fragment contains the dimerized insulator, the WAP promoter and signal sequence, the BChE-encoding sequence, and WAP coding and 3′ genomic regions (See FIG. 10).

### Example 8

### Expression Constructs for the Production of Recombinant BChE in the Urine of Transgenic Mammals

8.1. Uromodulin Promoter

Uromodulin, a 90 kD glycoprotein secreted from the epithelial cells of the thick ascending limbs and the early distal convoluted tubule in the kidney, is the most abundant protein in urine and is evolutionarily conserved in mammals [Badgett and Kumar, Urologia Internationalis (1998) 61:72-75]. Thus, the uromodulin promoter is a good candidate for driving the production of recombinant proteins in cells of the kidney, which will then secrete said proteins into the urine.

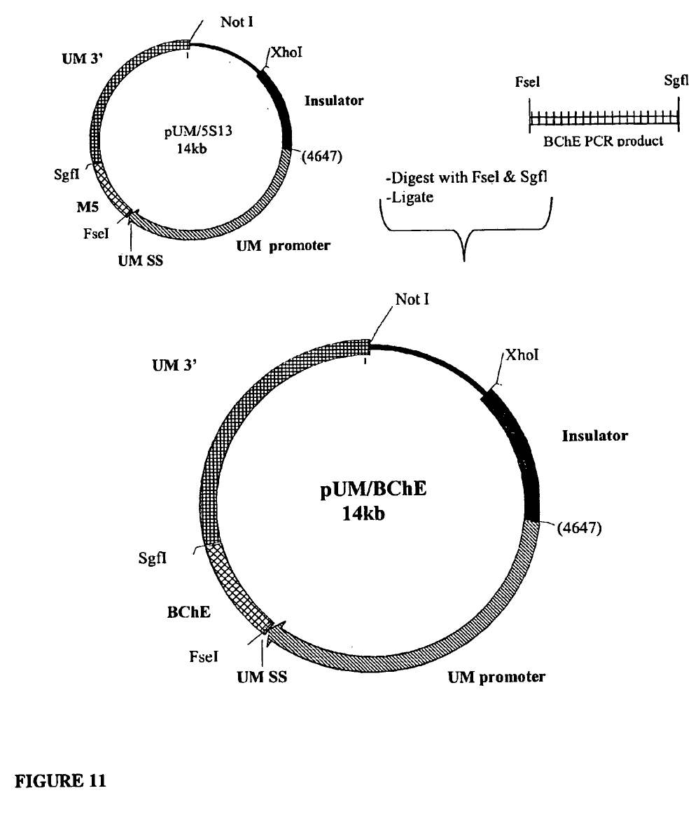

An expression construct comprising a uromodulin promoter and encoding a spider silk protein, pUM/5S13, may be used for the construction of a new expression construct, pUM/BChE, in which the expression of a BChE encoding sequence is controlled by the uromodulin promoter (See FIG. 11). The parent pUM/5S13 expression construct contains, in this order:

A 2.4 kb fragment of the chicken β-globin insulator;

A 3.4 kb fragment of the goat uromodulin promoter and signal sequence

A site for the restriction endonuclease FseI;

Sequences encoding a spider silk protein;

A site for the restriction endonuclease SgfI; and

A 2.8 kb fragment uromodulin 3′ genomic DNA.

The pUM/5S3 construct is digested with FseI and SgfI to remove the sequence encoding the spider silk protein. Please refer to PCT publication No. WO0/15772 (insulator and uromodulin promoter and genomic DNA elements), as well as Lazaris, et al. Science (2002) 295: 472-476 and PCT publication No. WO99/47661 (spider silk protein constructs), for disclosure of methods to construct pUM/5S13.

PCR is performed on a BChE cDNA clone (ATCC, #65726) with a sense primer (5′CAA TCA GGC CGG CCA GAA GAT GAC ATC ATA ATT GC-3′), (SEQ ID NO: 35) containing an FseI site (underlined) and an antisense primer (5′ CTA TGA CTC GAG GCG ATC GCT ATT MT TAG AGA CCC A CAC-3′) (SEQ ID NO: 10) including a SgfI site (underlined) to amplify the sequence encoding the mature human BChE protein.

This PCR product is digested with FseI and SgfI, and ligated with the FseI and SgfI fragment of pUM/5S13 to replace the spider silk encoding sequence with the BChE encoding sequence. This new construct is named pUM/BChE.

For microinjection or transfection, XhoI and NotI digestion of pUM/BChE removes the vector backbone and generates a linear DNA fragment. This fragment consists of the insulator, the uromodulin promoter and signal sequence, the BChE-encoding sequence, and a uromodulin 3′ genomic DNA fragment.

8.2. Uroplakin II Promoter

A group of membrane proteins known as uroplakins are produced on the apical surface of the urothelium. The term “urothelium” refers collectively to the epithleial lining of the ureter, bladder, and urethra. These uroplakin proteins form two-dimensional crystals, known as “urothelial plaques”, which cover over 80% of the apical surface of urothelium (Sun, et al. Mol. Biol. Rep. (1996) 23:3-11; Yu, et al. J. Cell Biol. (1994) 125:171-182). These proteins are urothelium-specific markers, and are conserved during mammalian evolution (Wu, et al. J. Biol. Chem. (1994) 269:13716-13724).

Transgenic mice that express human growth hormone (hGH) under the control of the mouse uroplakin II gene promoter have been generated. These mice express the recombinant hGH in the urothelium, and secrete the recombinant hGH into their urine at a concentration of 100-500 mg/l (Kerr, et al. Nat. Biotechnol. (1998) 16:75-79). This study is apparently the first report of using urothelium as a bioreactor for the production and secretion of bio-active molecules. It has subsequently been shown that urothelial cells are involved in urinary protein secretion (Deng, et al. Proc. Natl. Acad. Sci. USA (2001) 98:154-159).

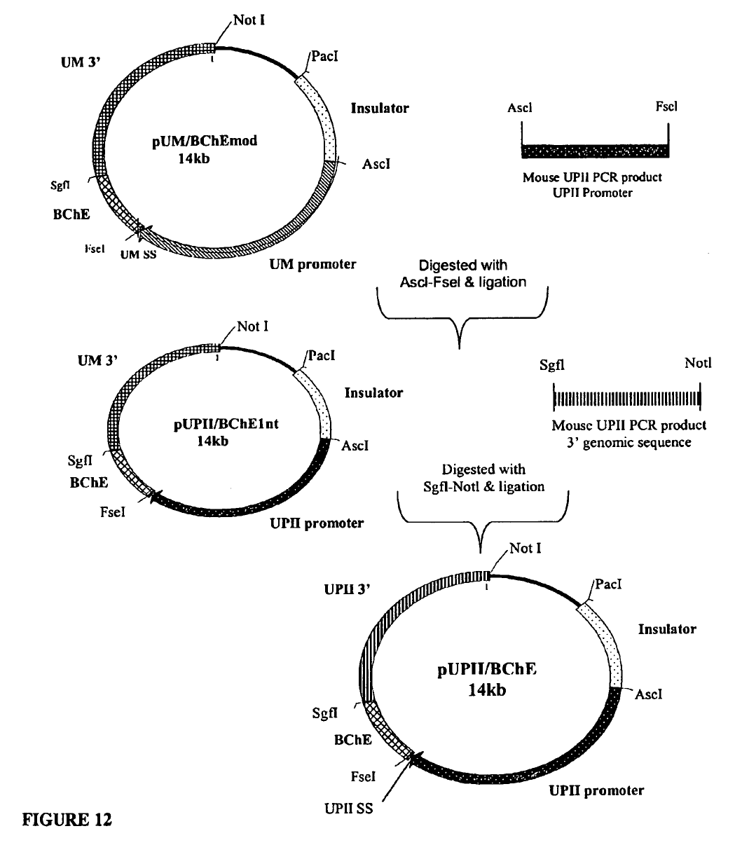

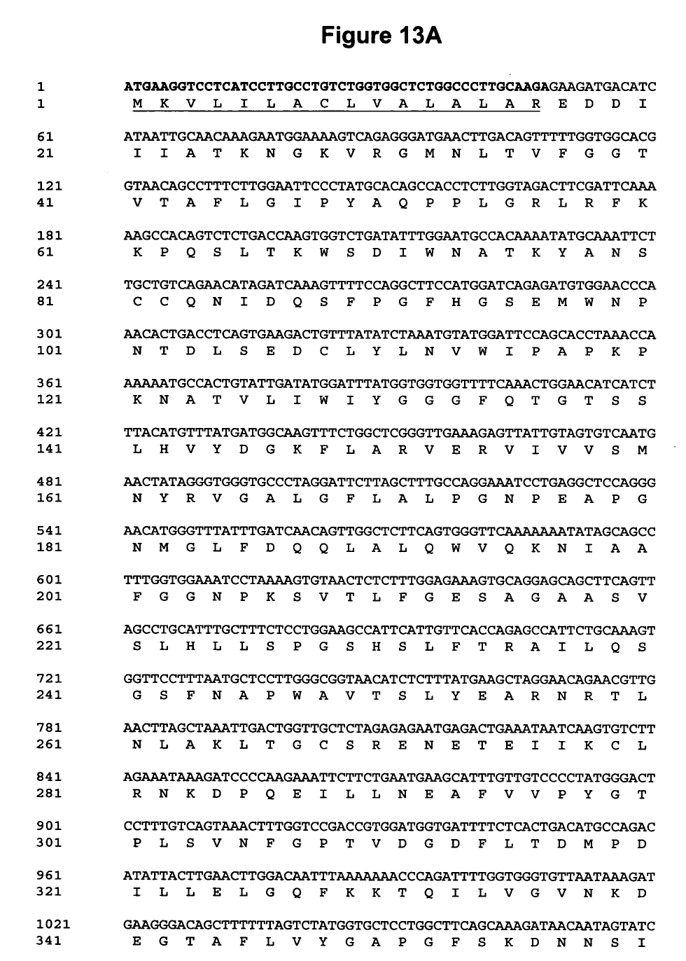

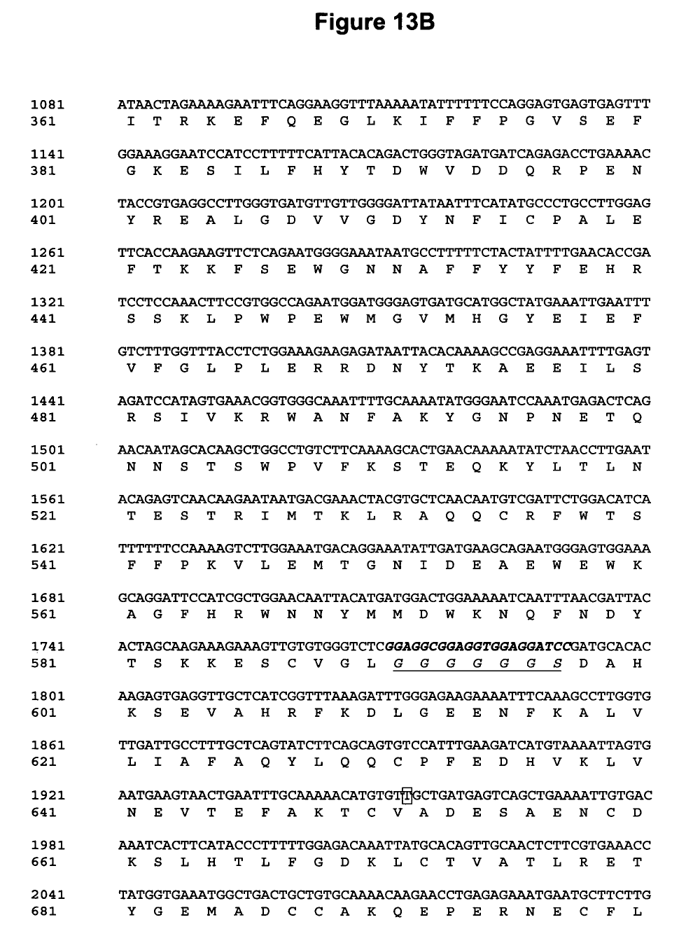

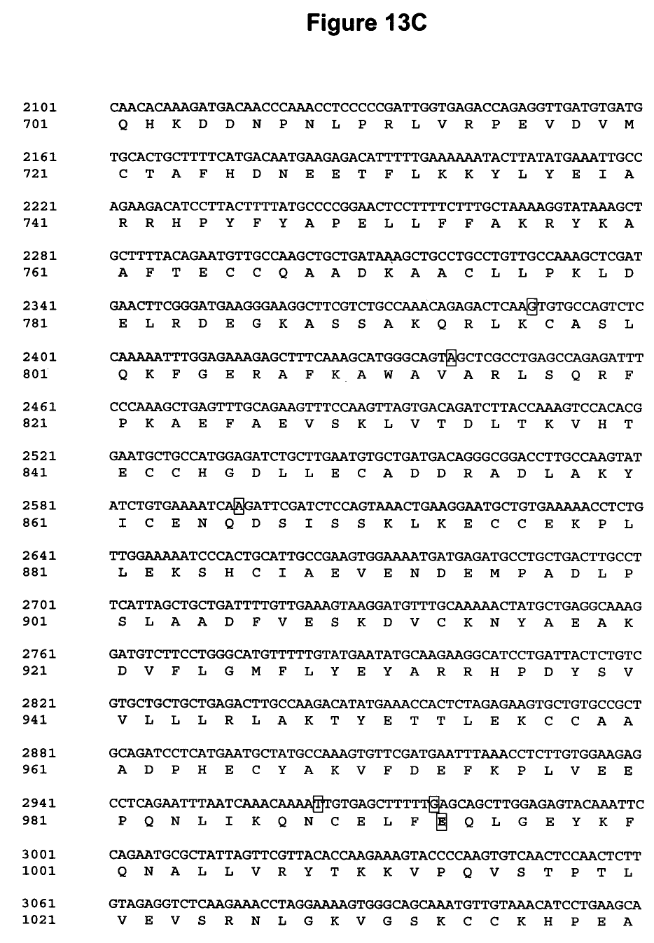

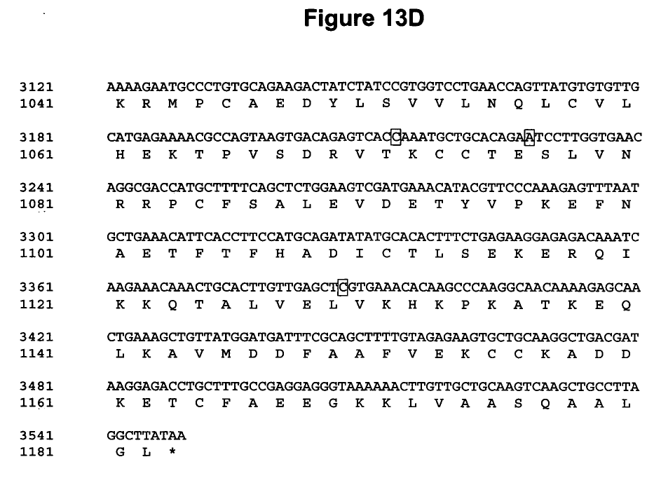

The expression construct pUM/BChE, comprising the uromodulin promoter and sequences encoding a BChE enzyme (See Example 8.1), may be modified for the construction of the new expression construct pUPII/BChE (See FIG. 12). The pUM/BChE expression construct contains, in this order: an 2.4 kb fragment of the chicken β-globin insulator; a 3.4 kb fragment of the goat uromodulin promoter and signal sequence; a site for the restriction endonuclease FseI; a BChE-encoding sequence; a site for the restriction endonuclease SgfI; and a 2.8 kb fragment of uromodulin 3′ genomic sequence.

Restriction endonuclease sites are introduced at the 5′ end (Pacd) and the 3′ end (AscI) of the chicken β-globin insulator sequence of pUM/BChE by conventional PCR to yield pUM/BChEmod.

PCR is performed on mouse genomic DNA with a sense primer (5′CAA TCA GGC GCG CCC TCG AGG ATC TCG GCC CTC TTT CTG 3′) (SEQ ID NO: 36) containing an AscI site (underlined) and an antisense primer (5′CAA TCA GGC CGG CCG CAA TAG AGA CCT GCA GTC CCC GGA G 3′) (SEQ ID NO: 37) including a FseI site (underlined) and partial sequence for the signal peptide of the uroplakin II protein. This PCR amplifies a DNA fragment containing the uroplakin II promoter plus the uroplakin signal sequence.

The uroplakin II PCR product is digested with AscI and FseI, and ligated with AscI and FseI digested pUMBChE to replace the goat uromodulin promoter with the mouse uroplakin II promoter. This step generates the construct pUPII/BChEInt.

A PCR is performed on mouse genomic DNA with a sense primer (5′CAT CTG GCG ATC GCT ACC GAG TAC AGA AGG GGA CG-3′) (SEQ ID NO: 38) containing a SgfI site (underlined) and an antisense primer (5′CTA GCA TGC GGC CGC GTG CTC TAG GAC AGC CAG AGC-3′) (SEQ ID NO: 39) containing a NotI site (underlined) to amplify a portion of the uroplakin II genomic sequence. This PCR product spans uroplakin II genomic sequence from within exon 4 through the 3′ end of the gene, including the polyA sequence. This PCR product is digested with SgfI and NotI, and then ligated to SgfI and NotI digested pUPII/BChEInt. This step replaces the goat uromodulin 3′ genomic sequences with mouse UPII 3′ genomic sequences to generate the final expression construct pUPII/BChE.

For microinjection or transfection, pUPII/BChE is linearized by PacI and NotI to remove the vector backbone. This linear fragment consists of the insulator, the uroplakin II promoter and signal sequence, a BChE-encoding sequence, and a uroplakin II 3′ genomic fragment.

### Example 9

### Production of Recombinant BChE-hSA Fusion Protein in Cell Culture

9.1 Assembly of Expression Constructs

Standard recombinant DNA methods employed herein have been described in detail (see, for example, in “Molecular Cloning: A Laboratory Manual.” 2nd Edition. Sambrook, et al. Cold Spring Harbor Laboratory:1989, “A Practical Guide to Molecular Cloning” Perbal: 1984, and “Current Protocols in Molecular Biology” Ausubel, et al., eds. John Wiley & Sons:1989). All DNA cloning manipulations were performed using E. coli STBII competent cells (Canadian Life Science, Burlington, Canada). Restriction and modifying enzymes were purchased from New England BioLabs (Mississauga, ON, Canada). All chemicals used were reagent grade and purchased from Sigma Chemical Co (St. Louis, Mo.), and all solutions were prepared with sterile and nuclease-free WFI water (Hyclone, Tex.). Construct integrity was verified by DNA sequencing analysis provided by McMaster University (Hamilton, ON, Canada). Primers were synthesized by Sigma Genosys (Oakville, ON, Canada). PCR was performed using Ready-To-Go PCR beads (Pharmacia Biotech, Baie d'Urf, PQ, Canada) or the High Fidelity PCR kit (Roche Diagnostics Canada, Laval, Canada).

In the expression contructs for the expression of recombinant BChE-hSA fusion protein in in vitro cell culture, a sequence encoding human BChE-hSA fusion protein was under the transcriptional control of a strong constitutive promoter and was linked to a signal sequence to provide secretion of the recombinant protein from the cells.

pCMV/BChE-hSA

PCR was performed using pCMV/BChE as a template with a sense primer Acb710 (5′ GTG TAA CTC TCT TTG GAG AAA G 3′) (SEQ ID NO: 5) containing a portion of 5′ BChE sequence and an antisense primer Acb853 (5′ TAT AAG TTT AAA CAT ATA ATT GGA TCC TCC ACC TCC GCC TCC GAG ACC CAC ACA ACT TTC TTT CTT G 3′) (SEQ ID NO: 6) containing a PmeI site (underlined), a BamHI site (italic), a (Gly)₆-Ser linker (bolded) followed by a portion of 3′ BChE sequence. The PCR product was digested with XbaI and PEmeI, and ligated to XbaI and PmeI digested pCMV/BChE to generate pCMV/BChEmd.

PCR was performed using Marathon-ready human liver cDNA pool (Clontech) as a template with a sense primer Acb854 (5′ ATA TAA GGA TCC GAT GCA CAC AAG AGT GAG GTT GCT CAT C₃′) (SEQ ID NO: 7) containing a BamHI site (underlined) and partial sequence from the hSA cDNA 5′ end (GenBank #V00495, without the signal sequence), and an antisense primer Acb855 (5′ ATT TAA GTT TAA ACT CAT TAT AAG CCT AAG GCA GCT TGA CTT GC 3′) (SEQ ID NO: 8) including a PmeI site (underlined) and partial sequence from the hSA cDNA 3′ end. This PCR product was digested with BamHI and PmeI and inserted into BamHI and PmeI digested pCMV/BChEmd to generate the final construct, pCMV/BChE-hSA. This expression construct encodes a BChE-hSA fusion protein.

9.2. Transfection and Selection of Stable Cell Lines

Preparation of Expression Constructs for Transfection:

The construct pCMV/BChE-hSA were digested with FspI, and the resultant FspI-digested linear DNA, was prepared and used for transfection. Briefly, circular expression construct DNA was purified by the cesium chloride gradient technique. This purified DNA was restricted with FspI, precipitated, and resuspended in sterile deionized water.

Stably Transfected MAC-T Cell Lines Expressing a Recombinant BChE-hSA Fusion:

MAC-T cells were seeded at a density of 2.5×10⁵ cells per 100 mm dish. On the following day, cells were transfected with Lipofectamine Reagent (Invitrogen) with 10 μg of the linearized pCMV/BChE-hSA construct. Briefly, the DNA was diluted to a final volume of 500 μL with DMEM (Invitrogen) and 60 μL of Lipofectamine was diluted to a final volume of 500 μL with DMEM. The two solutions were combined, vortexed for 10 sec and the complexes were allowed to form at room temperature for 30 min. DMEM was added to the lipid-DNA mixture up to a final volume of 5 ml. The mixture was then applied to the cells and allowed to incubate overnight at 37° C. under 5% CO₂. The cells were then cultured for another 24 h in DMEM containing 10% FBS, 5 μg/ml insulin (Sigma).

Stably transfected cells were selected in DMEM containing 10% FBS, 5 μg/ml insulin (Sigma), and 100 μg/ml hygromycin B (Invitrogen). Colonies surviving selection were picked 7 to 14 days following transfection and expanded further.

The level of BChE activity in cell culture media from pCMV/BChE-hSA transfected MAC-T cells was evaluated using a commercially available test (Sigma). From over 100 clones tested, the one demonstrating the highest BChE activity was further evaluated in roller bottles containing serum-free DMEM. The amount of BChE activity under these conditions was estimated at 0.17 units per million cells (U/10⁶) per 24 hours. Thus, it was successfully demonstrated that the recombinant BChE-hSA fusion protein is active.

Stably Transfected BHK Cell Lines Expressing a Recombinant BChE-hSA Fusion:

These lines were generated using the same procedure for stable transfection of MAC-T cells with pCMV/BChE-hSA, with the exception that the cells were BHK (Baby Hamster Kidney) cells (supplied by Dr. G. Matleshewski of McGill University, also available from the ATCC, clone #CCl-10) and the selection media contained DMEM with 10% FBS and 300 μg/ml hygromycin B (Invitrogen). Colonies surviving selection were picked 7 to 14 days following transfection and expanded further.

The level of BChE activity in cell culture media from pCMV/BChE-hSA transfected BHK cells was evaluated using a commercially available test (Sigma). From over 100 clones tested, the one demonstrating the highest BChE activity was further evaluated in roller bottles containing serum-free DMEM. The amount of BChE activity under these conditions was estimated at 0.73 units per million cells (U/10⁶) per 24 hours. A master cell bank was generated and used to initiate a hollow fiber bioreactor production run (Biovest, CP2500 model). Hollow fiber production of stable transfectants was established for large-scale production of recombinant BChE-hSA. Secretion level of the fusion protein in vitro was ˜30 mg/L.

9.3. Detection of Recombinant BChE-hSA Fusion Protein in Culture Media of Transfected Cells.

Western blotting analysis of non-denaturing PAGE gels and denaturing SDS-PAGE gels was used to detect the presence of recombinant BChE in cell culture media. Cell culture media from pCMV/BChE-hSA transfected MAC-T or BHK cells, was electrophoresed on non-denaturing and denaturing pre-cast 4-20% TRIS-glycine gels (Invitrogen). The samples were then transferred by electroblotting onto nitrocellulose membranes (Bio-Rad). Recombinant BChE-hSA fusion protein on the membranes was detected using rabbit polyclonal antibodies raised against BChE (DAKO) at a dilution of 1:1000 and goat anti-rabbit horseradish peroxidase conjugated second antibody. Detection was performed according to manufacturer's protocol for enhanced chemiluminescence (ECL) detection (Amersham Pharmacia).

In such analyses, the anti-BChE antibodies specifically detected a protein of the appropriate molecular weight in cell culture media from transfected cells. These results confirmed the production of the recombinant BChE-hSA fusion protein, in transfected cell lines in in vitro culture.

9.4. BChE-hSA Fusion Protein Activity Gels

20 μL of samples of cell culture media from pCMV/BChE-hSA transfected MAC-T and BHK cells, was electrophoresed on native 4-20% pre-cast TRIS-glycine gels at 100-125 V overnight and at 4° C. The gels were then stained for BChE activity with 2 mM of butyrylthiocholine iodide according to the Karnovsky and Roots method (Karnovsky and Roots, Histochem. Cytochem. (1964) 12:219-221). The staining procedure was performed at ambient temperature for two to six hours until the active protein bands were revealed.

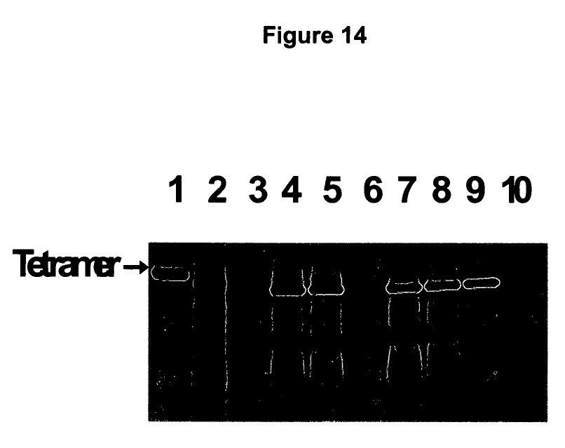

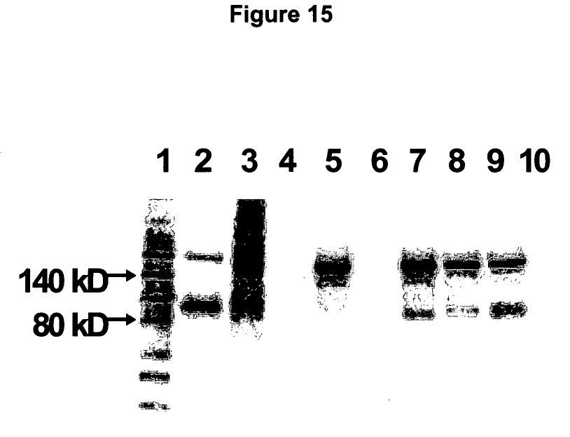

Conditioned media from MAC-T and BHK cells transfected with pCMV/BChE-hSA showed revealed that the fusion protein secreted in the cell media consisted of a predominant bioactive species (FIG. 3, lanes 3 and 5; FIG. 14, lane 3). This form was confirmed to have the expected molecular mass of approximately 150 kDa when analyzed by Western blot (FIG. 15, lane 3).

9.5. Purification of BChE-hSA

All the purification procedures were performed at 4° C. unless otherwise noticed. ˜30 L of BHK cell media were harvested from the hollow fiber system for purification (See Example 9.2). The media were mixed with 50%-saturated ammonium sulfate (AS) slowly until it dissolved, incubated for 1 h and centrifuged at 12,000 g for 30 min. Supernatant was collected, mixed with 80%-saturated AS slowly until it dissolved and incubated for 1 h prior to centrifugation at 12,000 g for 30 min. The pellet was resuspended in buffer A (20 mM sodium phosphate, pH 7.4, 1 mM EDTA) and dialyzed overnight against buffer B (buffer A+100 mM NaCl). The dialysate was loaded onto a procainamide affinity column equilibrated in buffer B. The column was washed with 10 bed volumes of buffer C (buffer A+150 mM NaCl), and eluted with buffer D (buffer A+300 mM NaCl). Fractions were assayed for BChE activity and pooled. The pool was dialyzed overnight against buffer A and stored at 4° C. For the BChE-hSA purification from the milk of transgenic goats, a methodology was developed based on precipitation of contaminating proteins with AS, followed by anion exchange chromatography and affinity column chromatography (procainamide). The purity of the material was assessed using PAGE stained with a silver staining kit (Bio-Rad).

9.6. Pharmaco-Kinetic Studies on BChE-hSA

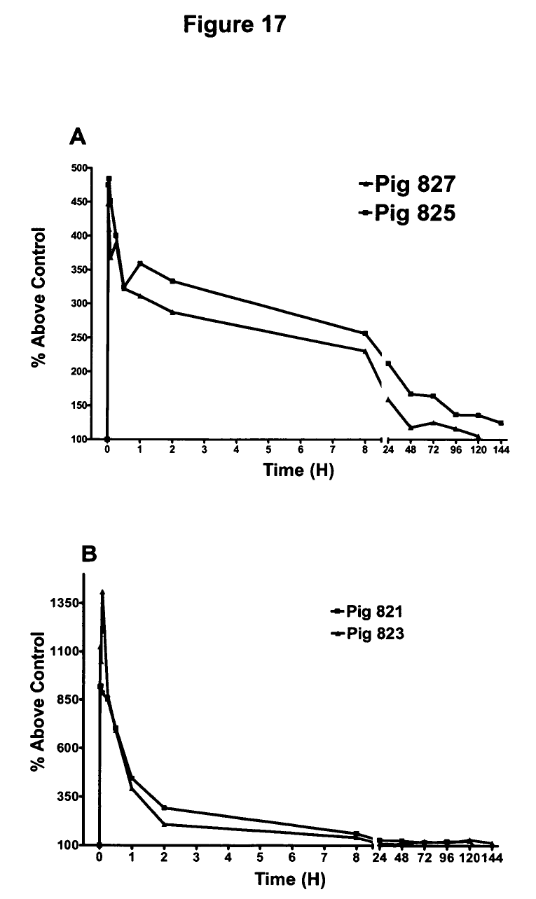

Purified BChE-hSA fusion protein produced from BHK cells was injected i.v. into juvenile swine for pharmaco-kinetic studies. Analysis of series blood samples using the Ellman assay revealed a substantial enhancement of the mean residence time of the fusion protein (˜32 h) when compared with a transgenically produced ruminant huBChE preparation containing >70% tetramer (˜3 h) (FIG. 17).

### Example 10

### Production of Recombinant Human BChE-hSA Fusion Protein in Transgenic Mice

10. Expression Constructs BCNN/BChE-hSA and Neo-BCNN/BChE-hSA

The goat β-casein promoter, including sequences through exon 2, were reverse PCR amplified from a genomic DNA library (SphI restriction digest) generated using goat blood (Clontech Genome Walking Library), using primers ACB582 (5′CAG CTA GTA TTC ATG GAA GGG CAA ATG AGG 3′) (SEQ ID NO: 41) and ACB591 (5′ TAG AGG TCA GGG ATG CTG CTA AAC ATT CTG 3′) (SEQ ID NO: 42). The 6.0 kb product was subcloned into the pUC18 vector (Promega) and designated pUC18/5′bCN.

A 4.5 kb DNA fragment spanning exon 7 and the 3′ end of the goat β-casein gene was reverse PCR amplified from the same library (BglII restriction digest) using primers ACB583 (5′CCA CAG MT TGA CTG CGA CTG GAA ATA TGG 3′) (SEQ ID NO: 43) and ACB601 (5′CTC CAT GGG TAA GCC TAA ACA TTG AGA TCT 3′) (SEQ ID NO: 44). The fragment was subcloned in the pUC18 vector as designated pUC18/3′bCN.

The 4.3 kb fragment encompassing exon 7 and the 3′ end of the goat β-casein gene was then PCR amplified from pUC18/3′bCN, using primer ACB620 (5′CTT TCT CAG CCC AAA GTT CTG CCT GTT C₃′) (SEQ ID NO: 45), which introduces NotI and XhoI sites and primer ACB621 (5′CAA GTT CTC TCT CAT CTC CTG CTT CTC A 3′) (SEQ ID NO: 46), which introduces SalI and Not I sites. This fragment was subcloned into the pUC18 vector and designated pUC18bCNA.

A 4.9 kb fragment containing the 5′ end of the β-casein promoter including sequences through exon 2 was PCR amplified from pUC18/5′bCN using primer ACB618 (5′CAG TGG ACA GAG GAA GAG TCA GAG GAA G 3′) (SEQ ID NO: 47), which introduces a BamHI and SacI site at the 5′ end and primer ACB619 (5′ GTA TTT ACC TCT CTT GCA AGG GCC AGA G 3′) (SEQ ID NO: 48), which is near the starting ATG codon and introduces a XhoI site. This fragment was then subcloned into the pUC18bCNA expression vector by digesting with XhoI, which digests at the 5′ end of the 3′ bCN fragment and BamHI, which is present in the pUC18 vector just upstream of the XhoI site. This ligation generates the final pUC18/BCNN construct, which contains the β-casein promoter, including sequences upto exon 2, followed by an XhoI site, exon 7 and the 3′ end of the β-casein gene.

The human BChE cDNA was PCR amplified from a cDNA clone (ATCC #65726) with a sense primer Acb719 (5′ ATA TTC TCG AGA GCC ATG AAG GTC CTC ATC CTT GCC TGT CTG GTG GCT CTG GCC CTT GCA AGA GAA GAT GAC ATC AT 3′) (SEQ ID NO: 9) containing an XhoI restriction endonuclease site (underlined), goat β-casein signal sequence (italic), and a partial human BChE sequence; and an antisense primer, Acb718 (5′CTA TGA CTC GAG GCG ATC GCT ATT AAT TAG AGA CCC ACA C3′) (SEQ ID NO: 10) containing an XhoI site (underlined) and partial 3′ human BChE sequence. The BChE PCR product was XhoI digested and subcloned into pGEM-T easy vector (Promega), to given the construct named p73. The BChE insert of p73 was excised by digestion with XhoI, purified with GFX matrix (Pharmacia Biotech, Baie d'Urf, PQ, Canada) and ligated with XhoI-digested pUC18/BCNN to generate pBCNN-BChE. The generation of pBCNN/BChE is shown schematically in FIG. 4.

The vector pUC18/BChE (FIG. 4) was digested with XhoI to remove the BChE insert, blunt-ended by filling in with Klenow polymerase in the presence of dNTPs, and CIP treated. Construct pCMV/BChE-hSA (See Example 9.1) was partially digested with NcoI to remove the BChE-hSA encoding sequences, blunt-ended by filling in with Klenow polymerase in the presence of dNTPs, and PmeI digested. The two blunt-ended fragments were ligated to generate BCNN/BChE-hSA. PCR cloning was performed to insert a neomycin resistance gene fragment between the insulators and the β-casein promoter in BCNN/BChE-hSA to generate Neo-BCNN/BChE-hSA for nuclear transfer. In these two constructs the signal sequence is the goat β-casein signal sequence.

BCNN/BChE-hSA and Neo-BCNN/BChE-hSA were digested with NotI, respectively, and the resultant NotI-digested linear DNA, free of bacterial sequences, was prepared and used to generate transgenic mice. Briefly, circular expression construct DNA was purified by the cesium chloride gradient technique. This purified DNA was restricted with NotI, electrophoresed, and the linear DNA fragment was gel purified. The DNA fragment was then mixed with cesium chloride and centrifuged at 20° C., 60,000 rpm for 16 to 20 hrs in a Beckman L7 ultracentrifuge using a Ti70.1 rotor (Beckman Instruments, Fullerton, Calif., USA). The DNA band was removed, dialyzed against WFI water for 2-4 hrs, and precipitated in ethanol. The precipitated DNA was resuspended in injection buffer (5 mM Tris pH 7.5, 0.1 mM EDTA, 10 mM NaCl) and dialyzed against the same buffer at 4° C. for 8 hrs. Two additional dialysis steps were performed, one for 16 hrs and the second for at least 8 hrs. After dialysis the DNA was quantitated using a fluorometer. Prior to use an aliquot was diluted to 2-3 ng/ml in injection buffer.

As a result of this preparation, the linear BCNN/BChE-hSA fragment used to generate transgenic animals contained, in this order:

Dimerized chicken β-globin gene insulator

Neomycin resistance gene fragment (for Neo-BCNN/BChE-hSA only)

Goat beta-casein promoter

β-casein exon 1;

β-casein intron 1;

Partial β-casein exon 2;

XhoI cloning site;

β-casein signal sequence;

BChE-hSA fusion protein encoding sequence;

A STOP codon;

Partial β-casein exon 7;

β-casein intron 7;

β-casein exon 8;

β-casein intron 8;

β-casein exon 9; and

Additional β-casein 3′ genomic sequence.

A schematic depicting the exons and introns of the goat β-casein locus that are contained in this fragment is shown in FIG. 5.

10.2 Production of Founders and Subsequent Generations of Transgenic Mice

The production and maintenance of transgenic mice were conducted at the McIntyre Transgenic Core Facility of McGill University. Transgenic mice were generated by pronuclear microinjection essentially as described in Hogan, et al. “Manipulating the Mouse Embryo: A Laboratory Manual.” Cold Spring Harbor Laboratory, 1986. The BCNN/BChE linear fragment was microinjected into fertilized eggs (strain FVB) and 31 pups were born.

At 2-3 weeks of age tail biopsies were taken, under anesthesia and DNA was prepared according to standard procedures well known to those skilled in the art, and described in detail, for example, in “Molecular Cloning: A Laboratory Manual.” 2nd Edition Sambrook, et al. Cold Spring Harbor Laboratory:1989). The presence of the transgene in genomic DNA was confirmed by PCR and/or Southern analysis as described in Identification of transgenic mice below. Out of 31 tail DNA samples, 3 founders (2 females and 1 male) were confirmed transgene positive. Southern analysis was also used to estimate transgene copy number.

10.3. Identification of Transgenic Mice

PCR Analysis:

Genomic DNA purified from tail biopsies was quantitated by fluorimetry and PCR screened using three different primer sets. PCR was performed with the Ready-To-Go PCR beads (Pharmacia Biotech). Upon amplification the samples were analysed for the presence of the PCR product by electrophoresis on a 2% agarose gel. The quality of the DNA used in these PCR reactions was confirmed by the presence of the expected fragment of the endogenous mouse β-casein gene.

Primer set A, primers ACB712 (5′CTT CCG TGG CCA GAA TGG AT 3′) (SEQ ID NO: 11) and ACB884 (5′CCT CAC TCT TGT GTG CAT CG 3′) (SEQ ID NO: 20), amplifies a 462 bp fragment from the 3′ end of the transgene spanning the junction of the BChE and albumin sequences.

Primer set B, ACB268 (5′ AGG AGC ACA GTG CTC ATC CAG ATC 3′) (SEQ ID NO: 13) and ACB659 (5′ GAC GCC CCA TCC TCA CTG ACT 3′) (SEQ ID NO: 14), amplifies an 893 bp fragment of the insulator sequence located at the 5′ end of the transgene.

Primer set C, ACB572 (5′ TTC CTA GGA TGT GCT CCA GGC T 3′) (SEQ ID NO: 15) and ACB255 (5′ GAA ACG GAA TGT TGT GGA GTG G 3′) (SEQ ID NO: 16) amplifies a 510 bp portion of an endogenous mouse β-casein gene. This primer set serves as in internal positive control to indicate that the extracted DNA can be amplified by PCR.

Southern Blotting Analysis:

Confirmation of transgene presence, and estimation of transgene copy number, was performed using Southern blotting analysis with Boehringer Mannheim's DIG system. DNA (5 μg) extracted from the tails of PCR positive transgenic mice were analysed by restriction enzyme digestion (HindIII) and Southern blotting using a nylon membrane (Roche Diagnostics). The membranes were hybridized with DIG Easy Hyb buffer at 42° C. overnight using a probe labeled by the PCR DIG probe synthesis kit (Roche Diagnostics). The probe was a 780 bp fragment of the BChE cDNA, located in the middle of the transgene (probe: ACB710/ACB819). The membranes were washed twice with 2×SSC—0.1% SDS at room temperature, 5 min each wash, and followed by two washes with 0.5×SSC—0.1% SDS at 65° C., 15 min each. Hybridization signals were detected using the CDP-Star™ substrate and visualized by the Fluor Chem™ 8000 System (Alpha Innotech Corporation).

10.4. Analysis of Recombinant BChE-hSA Fusion Protein in the Milk of Transgenic Mice

Lactating female mice were milked after induction with an intraperitoneal injection of 5 i.u. of oxytocin.

The milking apparatus is described online (www.invitrogen.com/Content/Tech-online/molecular_biology/manuals_pps/pbc1_man.pdf). The amount of milk that was obtained varied from 50-100 μl. The milk, collected from transgenic female mice, was analyzed using the Ellman assay and by analysis on non-denaturing PAGE gels stained for BChE activity and Western blot. There were no detectable levels of BChE in the F1 females from the male line, probably due to the low copy number of the transgene integrated. However, the results from the female line clearly indicated that BChE activity was present in the milk of the founder and her offspring (Table 2). Non-denaturing PAGE gels stained for BChE activity revealed that the fusion protein consisted of a predominant bioactive species (FIG. 14, lanes 4 and 5). This form was confirmed to have the expected molecular mass of approximately 150 kD when analyzed by Western blotting (FIG. 15 lanes 4 and 5).

### Example 11

### Production of Recombinant BChE-hSA Fusion Protein in Transgenic Goats

Trangenic goats expressing a recombinant BChE-hSA fusion protein may be generated by nuclear transfer. The nuclear donors are primary fetal goat cells stably transfected with the Neo-BCNN/BChE-hSA linear fragment (from Example 10.1).

11.1. Generation of Stably Transfected Cell Lines

Primary fetal goat cells were derived from day 28 kinder fetuses recovered from a pregnant Saanen breed female goat, and cultured for 3 days prior to being cyropreserved. Chromosome number (2n=60) and sex analysis was performed prior to use of cells for transfection experiments. Under the culture conditions used, all primary lines had a normal chromosome count indicating the absence of gross chromosomal instability during culture.

Transfections were performed as described in Keefer, et al. Biol. Reprod. (2001) 64:849-856, with the following modifications: Female primary lines were thawed and at passage 2, transfected with the linearized Neo-BCNN/BChE-hSA fragment. Stably transfected cell lines were selected with G418 and frozen by day 21 (day 0=transfection date).

Several stably transfected cell lines have been derived by this procedure. In all cases the presence of the transgene has been confirmed by Southern Analysis and by Fluorescence In Situ Hybridization (FISH). Transfected cell lines for which integration of the transgene is confirmed will serve as donors for nuclear transfer.

11.2. Oocyte Donor and Recipient Goats

Intravaginal sponges containing 60 mg of medroxyprogesterone acetate (Veramix) are inserted into the vagina of donor goats (Alpine, Saanen, and Boer cross bred goats) and left in place for 10 days. An injection of 125 μg cloprostenol is given 36 h before sponge removal. Priming of the ovaries is achieved by the use of gonadotrophin preparations, including FSH and eCG. One dose equivalent to 70 mg NIH-FSH-P1 of Ovagen is given together with 400 IU of eCG (Equinex) 36 h before LOPU (Laparoscopic Oocyte Pick-Up).

Recipients are synchronized using intravaginal sponges as described above for donor animals. Sponges are removed on day 10 and an injection of 400 IU of eCG is given. Estrus is observed 24-48 h after sponge removal and embryos are transferred 65-70 h after sponge removal.

11.3. Laparoscopic Oocyte Pick-Up (LOPU) and Embryo Transfer

Donor goats are fasted 24 hours prior to laparoscopy. Anesthesia is induced with intravenous administration of diazepam (0.35 mg/kg body weight) and ketamine (5 mg/kg body weight), and is maintained with isofluorane via endotrachial intubation. Cumulus-oocyte-complexes (COCs) are recovered by aspiration of follicular contents under laparoscopic observation.

Recipient goats are fasted and anaesthetized in the same manner as the donors. A laparoscopic exploration is performed to confirm if the recipient has had one or more recent ovulations or corpora lutea present on the ovaries. An average of 11 nuclear transfer-derived embryos (1-cell to 4-cell stage) are transferred by means of a TomCat catheter threaded into the oviduct ipsilateral to ovulation(s). Donors and recipients are monitored following surgical procedures and antibiotics and analgesics are administered according to approved procedures.

11.4. Oocyte Maturation

COCs are cultured in 50 μl drops of maturation medium covered with an overlay of mineral oil and incubated at 38.5-39° C. in 5% CO₂. The maturation medium consists of M199H (GIBCO) supplemented with bLH, bFSH, estradiol β-17, sodium pyruvate, kanamycin, cysteamine, and heat inactivated goat serum. After 23 to 24 hrs of maturation, the cumulus cells are removed from the matured oocytes by vortexing the COCs for 1-2 min in EmCare containing hyaluronidase. The denuded oocytes are washed in handling medium (EmCare supplemented with BSA) and returned to maturation medium. The enucleation process is initiated within 2 hr of oocyte denuding. Prior to enucleation, the oocytes are incubated in Hoechst 33342 handling medium for 20-30 minutes at 30-33° C. in air atmosphere.

11.5. Nuclear Transfer

Oocytes are placed into manipulation drops (EmCare supplemented with FBS) covered with an overlay of mineral oil. Oocytes stained with Hoechst are enucleated during a brief exposure of the cytoplasm to UV light (Zeiss Filter Set 01) to determine the location of the chromosomes. Stage of nuclear maturation is observed and recorded during the enucleation process.

The enucleated oocytes and dispersed donor cells are manipulated in handling medium. Transgenic donor cells are obtained following either in vitro transfection (see Example 3.1) or biopsy of a transgenic goat. Donor cells are prepared by serum starving for 4 days at confluency. Subsequently they are trypsinized, rinsed once, and resuspended in Emcore with serum. Small (<20 μm) donor cells with smooth plasma membranes are picked up with a manipulation pipette and slipped into perivitelline space of the enucleated oocyte. Cell-cytoplast couplets are fused immediately after cell transfer. Couplets are manually aligned between the electrodes of a 500 μm gap fusion chamber (BTX, San Diego, Calif.) overlaid with sorbitol fusion medium. A brief fusion pulse is administered by a BTX Electrocell Manipulator 200. After the couplets have been exposed to the fusion pulse, they are placed into 25 μl drops of medium overlaid with mineral oil. Fused couplets are incubated at 38.5-39° C. After 1 hr, couplets are observed for fusion. Couplets that have not fused are administered a second fusion pulse.

11.6. Oocyte Activation and Culture

Two to three hours after application of the first fusion pulse, the fused couplets are activated using calcium ionomycin and 6-dimethylaminopurine (DMAP) or using calcium ionomycin and cycloheximide/cytochalasin B treatment. Briefly, couplets are incubated for 5 minutes in EmCare containing calcium ionomycin, and then for 5 minutes in EmCare containing BSA. The activated couplets are cultured for 2.5 to 4 hrs in DMAP, then washed in handling medium and placed into culture drops (25 μl in volume) consisting of G1 medium supplemented with BSA under an oil overlay. Alternately, following calcium ionomycin treatment, the activated couplets are cultured for 5 hrs in cycloheximide and cytochalasin B, washed, and placed into culture. Embryos are cultured 12 to 18 hr until embryo transfer. Nuclear transfer derived embryos are transfered on Day 1 (Day 0=day of fusion) into synchronized recipients on Day 1 of their cycle (D0=estrus).

11.7. Identification of Stably Transfected Cell Lines and of Transgenic Goats

Following selection of transfected cell lines, genomic DNA is isolated from cell pellets using the DNeasy Tissue Kit (Qiagen, cat #69506). For each sample, the DNA is eluted in 150-200 μl 0.1× buffer AE and stored at 4° C. until ready to use.

For confirmation of the presence of the transgene in nuclear transfer derived offspring, genomic DNA is extracted from the blood and ear biopsy of 2 week old kids using standard molecular biology techniques. The genomic DNA is isolated from the blood samples using a QIAamp DNA Blood Mini Kit (Qiagen, Cat. # 51106), and from the tissue samples using DNeasy Tissue Kit (Qiagen, cat #69506). For each sample, the DNA is eluted in 150-200 μl 0.1× buffer AE and stored at 4° C. until use.

The presence of the transgene, in stably transfected cells and in transgenic goats, is confirmed by PCR as described in Example 2.3, except for the following modifications. Primer set C is replaced with the primers Acb256 (5′ GAG GAA CAA CAG CAA ACA GAG 3′) (SEQ ID NO: 21) and Acb312 (5′ ACC CTA CTG TCT TTC ATC AGC 3′) (SEQ ID NO: 22), which amplify a 360 bp portion of the endogenous goat β-casein gene. This primer set serves as in internal positive control to indicate that the extracted DNA can be amplified by PCR.

The presence of the transgene, in stably transfected cells and in transgenic goats, is also confirmed by Southern blotting as described in Example 2.3. Fluorescent in situ hybridization (FISH) is performed as described in Keefer, et al. Biol. Reprod. (2001) 64:849-856 in order to determine the number of chromosomal integration sites. The FISH probe contains only sequences from the insulator region of the transgene.

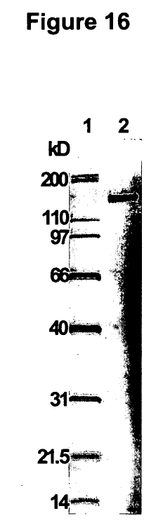

A total of 8 transgenic goats were generated using NT and two cell lines with the expression vector, Neo-BCNN/BChE-hSA, as determined by PCR, Southern blot and FISH analyses. These animals have 2-3 integration sites and contain a variety of transgene copy numbers in their respective genome. Of the 8 cloned transgenic goats 3 were induced hormonally into lactation. Goat 2217 expressed in average ˜1.5 g/L whereas the other two expressed ˜0.1 g/L, as determined by the Ellman assay (Table 2). Furthermore, goat 2217 was in natural lactation, producing 1 g/L of the fusion protein. ˜7.5 g of bioactive BChE/hSA were purified from approximately 8.7 L of milk. The purity as assessed by silver staining is estimated to be >90% (FIG. 16). Specific activity of the purified enzyme is ˜400 U/mg. This activity is as expected, approximately half of that of the BChE since the molecule is a hybrid of BChE and hSA. Non-denaturing PAGE gels stained for BChE activity and Western blot revealed that the fusion protein consisted of a predominant bioactive species with the expected molecular mass (FIG. 14, lanes 7, 8, 9; FIG. 15, lanes 7, 8, 9).

### Example 12

### BChE Expression Constructs Based on the WAP Promoter

12.1. Introduction

Whey acidic protein (WAP), the major whey protein in mammals, is expressed at high levels exclusively in the mammary gland during late pregnancy and lactation. The genomic locus of the murine WAP gene consists of 4.4 kb of 5′ flanking promoter sequence, 2.6 kb of coding genomic sequence, and 1.6 kb of 3′ flanking genomic DNA. The WAP promoter may be used to drive expression of heterologous proteins in the mammary gland of transgenic mammals [Velander, et al. Proc. Natl. Acad. Sci. USA (1992) 89: 12003-12007].

An expression construct based on the whey acidic protein (WAP) promoter, can be used to preferentially express BChE-hSA fusion protein in milk of transgenic animals. In one embodiment, the construct is assembled by inserting a BChE-hSA fusion protein-encoding sequence between the WAP promoter (position −949 to +33 nt) at the 5′ end, and the WAP coding genomic sequence (843 bp; the last 30 base of Exon 3, all of intron 3, and exon 4 including 70 bp of 3′ UTR) at the 3′ end. The expression construct also includes two copies of an insulator element from the chicken globin locus. The BChE-hSA fusion protein-encoding sequence may contain the BChE signal sequence or the WAP signal sequence. The BChE-hSA fusion protein-encoding sequence may also contain an epitope tag (e.g., myc and/or his).

In one embodiment, the contruct comprises the WAP gene promoter, the WAP signal sequence, a BChE-hSA fusion protein-encoding sequence, and the coding and 3′ genomic sequences of the WAP gene. This WAP signal sequence is added using a nucleic acid sequence encoding part of the 5′ untranslated region and the 19 amino acid signal peptide of the murine WAP gene (position −949 to +89, Hennighausen, et al. Nucl. Acids Res. (1982) 10:3733-3744). The BChE encoding fragment is generated by PCR of a BChE cDNA (e.g., ATCC #65726) using a 5′ primer containing the 90 bp sequence signal sequence flanked by a KpnI restriction endonuclease recognition site, and 3′ primers containing a KpnI restriction endonuclease recognition site and 3′ BChE cDNA sequences. The amplification is performed to maintain the correct reading frame. This PCR product is then inserted at the KpnI site at the first exon of WAP. The vector is prepared for microinjection or transfection by digestion with NotI restriction endonuclease and purification of the linear fragment.

12.2. Generation of the Expression Construct DWAP/BChE-hSA

The expression contruct pWAP/BChE-hSA may be prepared as follows:

Step 1: PCR Amplification of WAP 3′ Genomic Sequences

The WAP 3′ genomic sequence is PCR amplified from mouse genomic DNA with the following primers: WAP-p1 (5′ MT TGG TAC CAG CGG CCG CTC TAG AGG AAC TGA AGC AGA GAC CAT GC 3′) (SEQ ID NO: 23) and WAP-p2 (5′ GCT GCT CGA GCT TGA TGT TTA AAC TGA TAA CCC TTC AGT GAG CAG CCG ATA TAT GTT TAA ACA TGC GTT GCC TCA TCA GCC TTG TTC 3′) (SEQ ID NO: 24). The PCR product is then restricted with XhoI and NotI.

Step 2: PCR Amplification of WAP Coding Genomic Sequences

The WAP coding genomic sequence (2630 bp) is PCR amplified from mouse DNA with the primers WAP-p3 (5′ ATA TAT GTT TAA ACA TGC GTT GCC TCA TCA GCC TTG TTC 3′) (SEQ ID NO: 25) and WAP-p4 (5′ ATG TTC TCT CTG GAT CCA GGA GTG AAG G 3′) (SEQ ID NO: 26). The PCR product is then restricted with PmeI and BamHI.

Step 3: PCR Amplification of the BChE-hSA Fusion Protein Encoding Sequence

The BChE-hSA encoding sequence is PCR amplified from the pCMV/BChE-hSA (See Example 9.1) with the primers: a sense primer BChE-p1 (5′ ATT TCC CCG AAG TAT TAC 3′) (SEQ ID NO: 27) and an antisense primer Acb855 (5′ ATT TAA GTT TAA ACT CAT TAT AAG CCT AAG GCA GCT TGA CTT GC 3′) (SEQ ID NO: 8). The PCR product is then blunt ended.

Step 4: Ligation of the WAP Coding and 3′ Genomic Sequences with the BChE-hSA Fusion Protein Encoding Sequence

The pBluescript vector is restricted with KpnI and Sac II. A linker formed by annealing of the primer sequences Linker-p1 (5′ GGA CCG GTG TTA ACG ATA TCT CTA GAG CGG CCG CT 3′) (SEQ ID NO: 29) and Linker-p2 (5′CCG GAG CGG CCG CTC TAG AGA TAT CGT TAA CAC CGG TCC GC 3′) (SEQ ID NO: 30) is inserted to generate additional restriction enzyme sites (KpnI, NotI, XbaI, EcoRV, HpaI, AgeI and SaclI). The new vector is recircularized and then then restricted with EcoRV. The BChE-hSA fusion protein encoding PCR product of Step 3 is then blunt-ended, and ligated to this vector.

This new construct is restricted with XhoI and NotI, and the WAP 3′ genomic sequence PCR product from Step 1 is inserted. This construct is then restricted with PmeI and BamHI and the 2.6 kb WAP coding genomic sequence PCR product of Step 2 is inserted, to generate a construct wherein the BChE-encoding sequence was linked at its 3′ end to the WAP coding and 3′ genomic sequences.

Step 5: PCR Amplification of the Chicken β-Globin Insulator Sequence

The insulator fragment is derived from PCR amplification of chicken genomic DNA with the primers Insulator-p1 (5′ TTT TGC GGC CGC TCT AGA CTC GAG GGG ACA GCC CCC CCC CAA AG 3′) (SEQ ID NO: 31) and Insulator-p2 (5′ TTT TGG ATC CGT CGA CGC CCC ATC CTC ACT GAC TCC GTC CTG GAG TTG 3′) (SEQ ID NO: 32). The PCR product is restricted in two independent reactions; one with NotI and XhoI, and one with BamHI and SalI. The two restricted fragments are then ligated together to generate a 2 kb dimerized insulator fragment with NotI and BamHI sites on either end.

Step 6: Ligation of the WAP Promoter Sequence with the Insulator Fragment

A pBluescript clone containing the 4.4 kb WAP promoter in the pBluescript plasmid [clone 483, described in Velander, et al. Proc. Natl. Acad. Sci. USA (1992) 89:12003-12007] is restricted with SaclI and Not I. A linker formed by annealing of the primer sequences Linker-p3 (5′ GGA CTA GTT GAT CAG CGG CCG CTA TAG GAT CC 3′) (SEQ ID NO: 33) and Linker-p4 (5′GGC CTG GAT CCT ATA GCG GCC GCT GAT CAA CTA GTC CGC 3′) (SEQ ID NO: 34) is inserted to generate a recircularized construct of the 4.4 kb WAP promoter containing additional restriction sites (SaclI, SpeI, BclI, NotI and BamHI). This new construct is then restricted with Not I and BamHI and ligated to the insulator fragment from Step 5.

Step 7: Generation of pWAP/BChE-hSA

The BChE-hSA/WAP coding and 3′ genomic sequence construct from Step 4 is then restricted with SaclI and AgeI. The 6.8 kb fragment containing the insulator and WAP promoter is isolated from the construct of Step 6 by restriction with SaclI and AgeI. These two fragments are ligated to form pWAP/BChE-hSA. This final construct contains the dimerized chicken β-globin gene insulator followed by the WAP 4.4 kb promoter, the BChE-hSA, and the WAP 2.6 kb coding and 1.6 kb 3′ genomic sequences (See FIG. 9).

For microinjection or transfection, pWAP/BChE-hSA is linearized by NotI digestion to remove the vector sequences. This linearized fragment contains the dimerized insulator, the WAP promoter and signal sequence, the BChE-hSA-encoding sequence, and WAP coding and 3′ genomic regions (See FIG. 10).

### Example 13

### Expression Constructs for the Production of BChE-hSA Fusion Protein in the Urine of Transgenic Mammals

13.1. Uromodulin Promoter

Uromodulin, a 90 kD glycoprotein secreted from the epithelial cells of the thick ascending limbs and the early distal convoluted tubule in the kidney, is the most abundant protein in urine and is evolutionarily conserved in mammals [Badgett and Kumar, Urologia Internationalis (1998) 61:72-75]. Thus, the uromodulin promoter is a good candidate for driving the production of recombinant proteins in cells of the kidney, which will then secrete said proteins into the urine.

An expression construct comprising a uromodulin promoter and encoding a spider silk protein, pUM/5S13, may be used for the construction of a new expression construct, pUM/BChE-hSA, in which the expression of a BChE-hSA fusion protein encoding sequence is controlled by the uromodulin promoter (See FIG. 11). The parent pUM/5 μl 3 expression construct contains, in this order:

A 2.4 kb fragment of the chicken β-globin insulator;

A 3.4 kb fragment of the goat uromodulin promoter and signal sequence

A site for the restriction endonuclease FseI;

Sequences encoding a spider silk protein;

A site for the restriction endonuclease SgfI; and

A 2.8 kb fragment uromodulin 3′ genomic DNA.

The pUM/5S13 construct is digested with FseI and SgfI to remove the sequence encoding the spider silk protein. Please refer to PCT publication No. WO00/15772 (insulator and uromodulin promoter and genomic DNA elements), as well as Lazaris, et al. Science (2002) 295: 472-476 and PCT publication No. WO99/47661 (spider silk protein constructs), for disclosure of methods to construct pUM/5S13.

PCR is performed on the pCMV/BChE-hSA (See Example 9.1) with a sense primer (5′CAA TCA GGC CGG CCA GAA GAT GAC ATC ATA ATT GC-3′), (SEQ ID NO: 35) containing an FseI site (underlined) and an antisense primer (5′CTA TGA CTC GAG GCG ATC ACT CAT TAT AAG CCT AAG GCA GCT TGA CTT GC_-3′) (SEQ ID NO: 51) including a Sgfl site (underlined) to amplify the sequence encoding the BChE-hSA fusion protein.

This PCR product is digested with FseI and SgfI, and ligated with the FseI and SgfI fragment of pUM/5S13 to replace the spider silk encoding sequence with the BChE-hSA fusion protein encoding sequence. This new construct is named pUM/BChE-hSA.

For microinjection or transfection, XhoI and NotI digestion of pUM/BChE-hSA removes the vector backbone and generates a linear DNA fragment. This fragment consists of the insulator, the uromodulin promoter and signal sequence, the BChE-hSA fusion protein-encoding sequence, and a uromodulin 3′ genomic DNA fragment.

13.2. Uroplakin II Promoter

A group of membrane proteins known as uroplakins are produced on the apical surface of the urothelium. The term “urothelium” refers collectively to the epithleial lining of the ureter, bladder, and urethra. These uroplakin proteins form two-dimensional crystals, known as “urothelial plaques”, which cover over 80% of the apical surface of urothelium (Sun, et al. Mol. Biol. Rep. (1996) 23:3-11; Yu, et al. J. Cell Biol. (1994) 125:171-182). These proteins are urothelium-specific markers, and are conserved during mammalian evolution (Wu, et al. J. Biol. Chem. (1994) 269:13716-13724).

Transgenic mice that express human growth hormone (hGH) under the control of the mouse uroplakin 11 gene promoter have been generated. These mice express the recombinant hGH in the urothelium, and secrete the recombinant hGH into their urine at a concentration of 100-500 mg/l (Kerr, et al. Nat. Biotechnol. (1998) 16:75-79). This study is apparently the first report of using urothelium as a bioreactor for the production and secretion of bio-active molecules. It has subsequently been shown that urothelial cells are involved in urinary protein secretion (Deng, et al. Proc. Natl. Acad. Sci. USA (2001) 98:154-159).

The expression construct pUM/BChE-hSA, comprising the uromodulin promoter and sequences encoding a BChE-hSA fusion protein (See Example 5.1), may be modified for the construction of the new expression construct pUPII/BChE-hSA. The pUM/BChE-hSA expression construct contains, in this order: a 2.4 kb fragment of the chicken β-globin insulator; a 3.4 kb fragment of the goat uromodulin promoter and signal sequence; a site for the restriction endonuclease FseI; a BChE-hSA fusion protein-encoding sequence; a site for the restriction endonuclease SgfI; and a 2.8 kb fragment of uromodulin 3′ genomic sequence.

Restriction endonuclease sites are introduced at the 5′ end (Pacd) and the 3′ end (Ascl) of the chicken β-globin insulator sequence of pUM/BChE by conventional PCR to yield pUM/BChEmod.

PCR is performed on mouse genomic DNA with a sense primer (5′CAA TCA GGC GCG CCC TCG AGG ATC TCG GCC CTC TTT CTG 3′) (SEQ ID NO: 36) containing an AscI site (underlined) and an antisense primer (5′CAA TCA GGC CGG CCG CAA TAG AGA CCT GCA GTC CCC GGA G 3′) (SEQ ID NO: 37) including a FseI site (underlined) and partial sequence for the signal peptide of the uroplakin II protein. This PCR amplifies a DNA fragment containing the uroplakin II promoter plus the uroplakin signal sequence.

The uroplakin II PCR product is digested with AscI and FseI, and ligated with AscI and FseI digested pUMBChE-hSA to replace the goat uromodulin promoter with the mouse uroplakin II promoter and generate the construct pUPII/BChE-hSAInt.

A PCR is performed on mouse genomic DNA with a sense primer (5′CAT CTG GCG ATC GCT ACC GAG TAC AGA AGG GGA CG-3′) (SEQ ID NO: 38) containing a SgfI site (underlined) and an antisense primer (5′CTA GCA TGC GGC CGC GTG CTC TAG GAC AGC CAG AGC-3′) (SEQ ID NO: 39) containing a NotI site (underlined) to amplify a portion of the uroplakin II genomic sequence. This PCR product spans uroplakin II genomic sequence from within exon 4 through the 3′ end of the gene, including the polyA sequence. This PCR product is digested with SgfI and NotI, and then ligated to SgfI and NotI digested pUPII/BChE-hSAInt. This step replaces the goat uromodulin 3′ genomic sequences with mouse UPII 3′ genomic sequences to generate the final expression construct pUPII/BChE-hSA.

TABLE 6
Summary of transgenic animals producing
the BChE/hSA fusion protein

| | | | Transgene | Inte- | Expression |
|---|---|---|---|---|---|
| Transgenic | | Gen- | copy | gration | in milk |
| animal | Animal ID | eration | number | site | (g/L) |
| Mice | 307-1F | F0 | 40 | ND | 0.28 |
| | 307-1A2F | F1 | 1-2 | ND | 0.34 |
| | 307-1A7F | F1 | 8-10 | ND | 0.32 |
| | 307-1A7A2F | F2 | 8-10 | ND | 0.24 |
| | 307-1A7A4F | F2 | 1-2 | ND | 0.44 |
| Goats | 2176F | F0 | 18-28 | 2 | 0.10 |
| | 2177F | F0 | 6-10 | 1-2 | 1.50 |
| | 2178F | F0 | 18-28 | 2-3 | 0.10 |

ND, not determined.

For microinjection or transfection, pUPII/BChE-hSA is linearized by PacI and NotI to remove the vector backbone. This linear fragment consists of the insulator, the uroplakin II promoter and signal sequence, a BChE-hSA fusion protein-encoding sequence, and a uroplakin II 3′ genomic fragment.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, product descriptions, publications, and protocols are cited throughout this application, the disclosures of which are incorporated herein by reference in their entireties for all purposes.

### CITED REFERENCES

Allon, et al. Toxicol. Sci. (1998) 43:121-128.
Altamirano, et al. J Neurochemistry (2000) 74:869-877.
Andres, et al. Proc Natl Acad Sci USA (1987) 84(5):1299-1303.
Arkin, et al. Proc. Natl. Acad. Sci. USA (1992) 89:7811-7815
Ashani, et al. Biochem Pharmacol (1991) 41:37-41.
Ausubel, et al., eds. Current Protocols in Molecular Biology (1989) John Wiley & Sons.
Badgeft and Kumar. Urologia Internationalis (1998) 61:72-75.
Baranyi, et al. Gene (1996) 174(1):27-34.
Blong, et al. Biochem J. (1997) 327:747-757.
Bon, et al. J. Biol. Chem. (1997) 272(5):3016-3021.
Brandeis, et al. Pharmacol Biochem Behav (1993) 46:889-896.
Broomfield, et al. J Pharmacol Exp Ther (1991) 259:633-638.
Cadwell, et al. PCR Methods and Applications (1992) 2:28-33.
Cascio, et al. Minerva Anestesiol (1998) 54:337.
Chitlaru, et al. Biochem. J. (1998) 336:647-658.
Cibelli, et al. Science (1998) 280: 1256-8.
Davis, J. M. ed. Basic Cell Culture 2nd Edition (2002). Oxford University Press.
Deng, et al. Proc. Natl. Acad. Sci. USA (2001) 98:154-159.
Ebert, et al. Biotechnology (1994) 12:699-702.
Ellman et al. Biochem Pharmacol (1961) 7:88-95.
Felgner, et al., eds. Artificial Self-assembling Systems for Gene Delivery (1996). Oxford University Press.
Greener, et al. Strategies in Mol. Biol. (1995) 7:32.
Gutierrez, et al. Transgenic Research (1996) 5(4):271-279.
Harduin-Lepers, et al. Biochimie (2001) 83:727-737.
Hennet. Cell Mol. Life Sci. (2002) 59:1081-1095
Hennighausen, et al. Nucl. Acids Res. (1982) 10:3733-3744.
Hermes, et al. Proc. Natl. Acad. Sci. USA (1990) 87:696-700.
Hoffman, et al. J Toxicol Clin Toxicol (1996) 34:259-266.
Hogan, et al. eds. Manipulating the Mouse Embryo: A Laboratory Manual (1986). Cold Spring Harbor Laboratory.
Jackson and Abbott, eds. Mouse Genetics and Transgenics: A Practical Approach (2000). Oxford University Press.
Huston et al., PNAS (1988) 85:5879-5883.
Kamovsky and Roots, Histochem. Cytochem. (1964) 12:219-221.
Keefer, et al. Biol. Reprod. (2001) 64:849-856.
Keefer, et al. Biol. Reprod. (2002) 66:199-203.
Kerr, et al. Nat. Biotechnol. (1998) 16:75-79.
Lazaris, et al. Science (2002) 295: 472-476.
Krejci et al. J. Biol. Chem. (1997) 272:22840-22847.
Lebkowski, et al. Mol Cell Biol (1988) 8(10):3988-3996.
Lee, et al. Nucleic Acids Res. (1988) 16:1027-1041.
Lockridge and La Du, J Biol Chem (1978) 253:361-366.
Lockridge, et al. Biochemistry (1997) 36:786-795.
Lockridge, et al. Biochemistry (1997) 36(4):786-795.
Lynch, et al Toxicol Appl Pharmacol (1997) 145:363-371.
Masson, et al. J Biol Chem (1993) 268(19):14329-41.
Masters, ed. Animal Cell Culture: A Practical Approach 3rd Edition (2000). Oxford University Press.
McClenaghan, et al. Biochem J (1995) 3 10(Pt2):637-641.
McKnight, et al. Proc Natl Acad Sci USA (1992) 89:6943-6947.
McKnight, et al. Mol Reprod Dev (1996) 44(2): 179-184.
McTiernan, et al. Proc. Natl. Acad. Sci USA (1987) 84:6682-6686.
Murphy, et al., Eds. Transgenesis Techniques (1993). Human Press, Totowa, N.J.
Murray, et al., eds. Transgenic Animals in Agriculture (1999). Oxford University Press.
Oliphant, et al. Gene (1986) 44:177-183.
Page, et al. Transgenic Res (1995) 4(6):353-360.
Perbal A Practical Guide to Molecular Cloning, 2nd Edition (1988). Wiley. Platteborze and Broomfield. Biotechnol Appl. Biochem (2000) 31:225-229.
Prody, et al. Proc. Natl. Acad. Sci. USA (1987) 84:3555-3559.
Puhler, Ed. Genetic Engineering of Animals (1993). A.VCH Verlagsgesellschaft, Weinheim, N.Y.
Raveh, et al. Biochem Pharmacol (1993) 45(12):2465-2474.
Raveh, et al. Toxicol. Appl. Pharmacol. (1997) 145:43-53.
Rosenberg, et al. Life Sciences (2002) 72:125-134.
Sambrook, et al. Molecular Cloning: A Laboratory Manual 2nd Edition (1989). Cold Spring Harbor Laboratory.
Saxena, et al. Biochemistry (1997) 36:7481-7489.
Saxena, et al. Molec. Pharmacol. (1998) 53:112-122.
Sikri, et al. Biochem. J. (1985) 225:481-486.
Soreq, et al. EMBO Journal 1984 3(6): 1371-1375.
Soreq, et al. J. Biol. Chem. (1989) 264:10608-10613.
Stacey, et al. Mol. Carcinog. (1990) 3:216-225.
Sun, et al. Mol. Biol. Rep. (1996) 23:3-11.
Syed et al., Blood (1997) 89:3243-3252.
Takashima, et al. J. Biol. Chem. (2002) 277:45719-45728.
Velander, et al. Proc. Natl. Acad. Sci. USA (1992) 89:12003-12007.
Vilotte, et al. Eur. J. Biochem. (1989) 186: 43-48.
Wang, et al. Molecular Reproduction and Development (2002) 63:437-443.
Wei, et al. Biochem Pharmacol (2000) 60(1): 121-126.
Wilmut, et al. Nature (1997) 385: 810-813.
Wolfe, et al Toxicol Appl Pharmacol (1992) 117(2):189-193.
Wu, et al. J. Biol. Chem. (1994) 269:13716-13724.
Xie, et al. Molec. Pharmacol. (1999) 55:83-91.
Yu, et al. J. Cell Biol. (1994) 125:171-182.
Yuan, et al. Acta Pharmacologica Sinica, (1999) 20:74-80.
Zbikowska, et al. Biochem J (2002) 365(Ptl):7-11.
Zbikowska, et al. Transgenic Res 2002 11 (4):425-435.

What is claimed is:

1. A method for producing a fusion protein that comprises an enzymatically active butyrylcholinesterase (BChE) enzyme and a human serum albumin (hSA), comprising expressing said fusion protein in a recombinant cell that comprises a polynucleotide encoding said fusion protein operably linked to a promoter sequence.

2. The method of claim 1, wherein said promoter sequence is the cytomegalovirus (CMV) promoter.

3. The method of claim 1, wherein said polynucleotide and operably linked promoter are part of a plasmid.

4. The method of claim 1, wherein said BChE and said hSA are separated by an oligopeptide linker that permits independent folding and activity of said BChE.

5. The method of claim 4, wherein said oligopeptide linker is a polyglycine and serine linker.

6. The method of claim 1, wherein said cell is a BHK cell.

7. The method of claim 1, wherein said BChE is human BChE.

8. The method of claim 1, wherein said polynucleotide further comprises a signal sequence that directs secretion of said fusion protein by the cell.

9. The method of claim 1, wherein said BChE-hSA fusion protein comprises the amino acid sequence of SEQ ID NO: 50.

10. The method of claim 1, wherein said polynucleotide comprises the nucleotide sequence of SEQ ID NO: 49

11. The method of claim 1, wherein said cell further comprises a DNA sequence encoding a glycosyltransferase, operably linked to a promoter.

12. The method of claim 11, wherein said glycosyltransferase is expressed by said cell and glycosylates the BChE portion of the fusion protein.

13. An isolated fusion protein, comprising an enzymatically active BChE enzyme and a human serum albumin.

14. The isolated fusion protein of claim 13, wherein said fusion protein further comprises a signal sequence that directs secretion of said fusion protein from a cell.

15. The isolated fusion protein of claim 13, wherein said fusion protein further comprises a linker located between said BChE enzyme and said hSA protein and wherein said linker permits independent folding and activity of said BChE.

16. The isolated fusion protein of claim 13, wherein said linker comprises an amino acid sequence.

17. The isolated fusion protein of claim 16, wherein said amino acid sequence comprises at least 7 amino acid residues.

18. The isolated fusion protein of claim 17, wherein said amino acid residues are glycine and serine residues.

19. The isolated fusion protein of claim 13, wherein said BChE is a human BChE.

20. The isolated fusion protein of claim 19, wherein said human BChE-hSA fusion protein comprises the amino acid sequence of SEQ ID NO: 50.

21. The isolated fusion protein of claim 13, wherein the BChE portion of said fusion protein comprises one or more glycosyl residues.

22. The isolated fusion protein of claim 14, wherein said signal sequence directs secretion into milk.

23. The isolated fusion protein of claim 14, wherein said signal sequence directs secretion into urine.

24. An isolated polynucleotide, comprising: (i) a nucleotide sequence encoding the fusion protein of claim 13, (ii) a promoter that directs expression of the fusion protein, and (iii) at least one signal sequence that directs secretion of the expressed fusion protein from a cell.

25. The isolated polynucleotide of claim 24, wherein the encoded fusion protein further comprises an oligopeptide linker.

26. The isolated polynucleotide of claim 25, wherein said oligopeptide linker comprises a polyglycine and a serine sequence.

27. The isolated polynucleotide of claim 24, wherein the BChE portion of said fusion protein is a human BChE.

28. The isolated polynucleotide of claim 27, wherein said human BChE-hSA fusion protein comprises the amino acid sequence of SEQ ID NO: 50.

29. The isolated polynucleotide of claim 24, wherein said polynucleotide comprises the nucleotide sequence of SEQ ID NO: 49.

30. The isolated polynucleotide of claim 24 wherein said signal sequence directs secretion into milk.

31. The isolated polynucleotide of claim 24, wherein said signal sequence directs secretion into urine.

32. The isolated polynucleotide of claim 24, wherein the promoter is a mammary gland-specific promoter selected from the group consisting of a WAP (whey acidic protein) promoter and a casein promoter.

33. The isolated polynucleotide of claim 24, wherein the promoter is a urinary endothelium-specific promoter selected from the group consisting of a uroplakin promoter or a uromodulin promoter.

34. A recombinant cell that comprises the isolated polynucleotide of claim 24.

35. The recombinant cell of claim 34, wherein the cell is a MAC-T (mammary epithelial) cell.

36. The recombinant cell of claim 24, wherein the cell is a BHK (baby hamster kidney) cell.

37. The recombinant cell of claim 24, wherein the cell is selected from the group of embryonic stem cells, embryonal carcinoma cells, primordial germ cells, oocytes, or sperm.

38. A non-human mammalian embryo that comprises the isolated polynucleotide of claim 24 as part of its genome.

39. A non-human mammalian embryo which comprises the polynucleotide of claim 32 as part of its genome.

40. A non-human mammalian embryo which comprises the polynucleotide of claim 33.

41. A non-human transgenic mammal that upon lactation, expresses in its milk the fusion protein of claim 13.

42. The non-human transgenic mammal of claim 41, wherein the linker portion of said fusion protein comprises at least 7 amino acid residues.

43. The non-human transgenic mammal of claim 42, wherein said amino acid residues are glycine and serine residues.

44. The non-human transgenic mammal of claim 41, wherein said mammal is a mouse.

45. The non-human transgenic mammal of claim 41, wherein said mammal is a goat.

46. A non-human transgenic mammal that upon urination, expresses in its urine the fusion protein of claim 13.

47. The non-human transgenic mammal of claim 46, wherein the linker portion of said fusion protein comprises at least 7 amino acid residues.

48. The non-human transgenic mammal of claim 47, wherein said amino acid residues are glycine and serine residues.

49. The non-human transgenic mammal of claim 46, wherein said mammal is a mouse.

50. The non-human transgenic mammal of claim 46, wherein said mammal is a goat.

51. A non-human transgenic mammal whose genome comprises the polynucleotide of claim 24.

52. The non-human transgenic mammal of claim 51, wherein the promoter is a mammary gland-specific promoter selected from the group consisting of a WAP (whey acidic protein) promoter and a casein promoter.

53. The non-human transgenic mammal of claim 51, wherein the genome of the mammal further comprises a DNA sequence encoding a glycosyltransferase, operably linked to a mammary gland-specific promoter, and a signal sequence that provides secretion of the glycosyltransferase.

54. The non-human transgenic mammal of claim 53, wherein the mammary gland-specific promoter is a casein promoter or a whey acidic protein (WAP) promoter.

55. The non-human transgenic mammal of claim 51, wherein the promoter is a urinary endothelium-specific promoter selected from the group consisting of a uroplakin promoter or a uromodulin promoter.

56. The non-human transgenic mammal of claim 51, wherein the genome of the mammal further comprises a DNA sequence encoding a glycosyltransferase, operably linked to a urinary gland-specific promoter, and a signal sequence that provides secretion of the glycosyltransferase.

57. The isolated polynucleotide of claim 56, wherein the promoter is a urinary endothelium-specific promoter selected from the group consisting of a uroplakin promoter or a uromodulin promoter.

58. The non-human transgenic mammal of claim 51, wherein said mammal is a mouse.

59. The non-human transgenic mammal of claim 51, wherein said mammal is a goat.

60. A method for producing a transgenic mammal that upon lactation secretes the fusion protein of claim 13 in its milk, which method comprises allowing an embryo, into which at least one genetically-engineered DNA sequence, comprising (i) a sequence encoding said fusion protein; (ii) a mammary gland-specific promoter; and (iii) a signal sequence that provides secretion of the fusion protein into the milk of the mammal, has been introduced, to grow when transferred into a recipient female mammal, resulting in the recipient female mammal giving birth to the transgenic mammal.

61. The method of claim 60, further comprising introducing the genetically-engineered DNA sequence into a cell of the embryo, or into a cell that will form at least part of the embryo.

62. The method of claim 61, wherein introducing the genetically-engineered DNA sequence comprises pronuclear or cytoplasmic microinjection of the DNA sequence.

63. The method of claim 61, wherein introducing the genetically-engineered DNA sequence comprises combining a mammalian cell stably transfected with the DNA sequence with a non-transgenic mammalian embryo.

64. The method of claim 61, wherein introducing the genetically-engineered DNA sequence comprises the steps of (a) introducing the DNA sequence into a non-human mammalian oocyte; and (b) activating the oocyte to develop into an embryo.

65. A method for producing a transgenic mammal that upon lactation secretes the fusion protein of claim 13, in its milk, which method comprises cloning or breeding of a transgenic mammal, the genome of which comprises a DNA sequence encoding said fusion protein, operably linked to a mammary gland-specific promoter, wherein the sequence further comprises a signal sequence that provides secretion of the fusion protein into the milk of the mammal.

66. A method for producing a transgenic mammal that secretes the fusion protein of claim 13, in its urine, which method comprises allowing an embryo, into which at least one genetically-engineered DNA sequence, comprising (i) a sequence encoding said fusion protein; (ii) a urinary endothelium-specific promoter; and (iii) a signal sequence that provides secretion of the fusion protein into the urine of the mammal, has been introduced, to grow when transferred into a recipient female mammal, resulting in the recipient female mammal giving birth to the transgenic mammal.

67. The method of claim 66, which further comprises introducing the genetically-engineered DNA sequence into a cell of the embryo, or into a cell that will form at least part of the embryo.

68. The method of claim 67, wherein introducing the genetically-engineered DNA sequence comprises pronuclear or cytoplasmic microinjection of the DNA sequence.

69. The method of claim 67, wherein introducing the genetically-engineered DNA sequence comprises combining a mammalian cell stably transfected with the DNA sequence with a non-transgenic mammalian embryo.

70. The method of claim 67 wherein introducing the genetically-engineered DNA sequence comprises the steps of (a) introducing the DNA sequence into a non-human mammalian oocyte; and (b) activating the oocyte to develop into an embryo.

71. A method for producing a transgenic mammal that secretes the fusion protein of claim 13, in its urine, which method comprises cloning or breeding of a transgenic mammal, the genome of which comprises a DNA sequence encoding said fusion protein, operably linked to a urinary endothelium-specific promoter, wherein the sequence further comprises a signal sequence that provides secretion of the fusion protein into the urine of the mammal.

72. A method for producing the fusion protein of claim 13, comprising: (a) inducing or maintaining lactation of a transgenic mammal, the genome of which comprises a DNA sequence encoding said fusion protein, operably linked to a mammary gland-specific promoter, wherein the sequence further comprises a signal sequence that provides secretion of the fusion protein into the milk of the mammal; and (b) extracting milk from the lactating mammal.

73. The method according to claim 72, which comprises the additional step of isolating the fusion protein from the extracted milk.

74. The method according to claim 73, further comprising purifying the fusion protein.

75. The milk of a non-human mammal comprising the fusion protein of claim 13.

76. The milk of claim 76, where the milk is whole milk.

77. The milk of claim 76, where the milk is defatted milk.

78. A method for producing the fusion protein of claim 13, comprising extracting urine from a transgenic mammal, the genome of which comprises a DNA sequence encoding said fusion protein, operably linked to a urinary endothelium-specific promoter, where the sequence further comprises a signal sequence that provides secretion of said fusion protein into the urine of the mammal.

79. The method according to claim 78, comprising the additional step of isolating the fusion protein from the extracted urine.

80. The method according to claim 79, further comprising purifying the fusion protein.

81. Urine of a non-human mammal comprising the fusion protein of claim 13.

82. A composition comprising the fusion protein of claim 13 in a pharmaceutically acceptable carrier.

83. A method for treating organophosphate poisoning, comprising administering to a subject in need thereof a therapeutically effective amount of a pharmaceutical composition of claim 82.

84. A method for the treatment of post-surgical, succinyl choline-induced apnea, which comprises administering to a subject in need thereof a therapeutically effective amount of a pharmaceutical composition of claim 82.

85. A method for the treatment of cocaine intoxication, which comprises administering to a subject in need thereof a therapeutically effective amount of a pharmaceutical composition of claim 82.
